SEQUENCE LISTING (124 sequences)

SEQ ID NO: 1 - protein, 138 aa, Burkholderia cenocepacia
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 2 - protein, 138 aa, Burkholderia gladioli
GSYTLGSYQISAPQLPAYNGQTVGTFYYVNDAGGLESRTFSSGGPTPYPNYANAGHVEGQSALFMRDNGISDGLVFHNNPEGTCGFCVNMTETLLPENSKLTVVPPEGSIPVKRGATGETRTFTGNSKSPKSPVKGGC

SEQ ID NO: 3 - protein, 140 aa, Caballeronia sp.
TETFPLGSNQIPRPGLPAYDGKTSGVFYYLDGDGGMQSKVFTSGGVTEFPNYANAGHVEGQSALFMRSNGVGEGVVFHNNPEGTCGFCVSMTETLLPENAKLTVVPPEGAVPVKRGATGDVRVFTGNKNSPKSPSSGGAC

SEQ ID NO: 4 - protein, 145 aa, Roseburia intestinalis
SQYPCKEEMSAGAGESGRKTISLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 5 - protein, 125 aa, Falcatimonas sp.
GGSSSINLPEYDGKTTHGVLVLDDGTQVPFSSGNANPNYKNYIPASHVEGKSAIYMRENGINNGTVFHNNTDGTCPYCDKMLPTLLEEGSTLTVVPPANANAPKPSWVDTVKTYIGNDKIPKKPK

SEQ ID NO: 6 - protein, 146 aa, Clostridium sp.
QSNMSITDRLAKQKEKQDNTNIIDNRPKLPDYDGKTTHGILVTPNSEHIPFSSGNPNPNYKNYIPASHVEGKSAIYMRENGITSGTIYYNNTDGTCPYCDKMLSTLLEEGSVLEVIPPINAKAPKPSWVDKPKTYIGNNKVPKPNK

SEQ ID NO: 7 - protein, 130 aa, Ruminococcus bicirculans
NANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSNSVANNVRAIPVPKTYIGNSTVPKIK

SEQ ID NO: 8 - protein, 136 aa, Burkholderia ubonensis
ALLREAYPSMEGATLPPFDGKTTIGLMFYTDASGQYQVKKLFSGEKVLSNYDATGHVEGKAALIMRNEKITEAVVMHNHPSGTCNYCDKQVETLLPKNATLRVIPPENAKAPTSYWNDQPTTYRGDGKDPKAPSKK

SEQ ID NO: 9 - protein, 133 aa, Treponema sp.
SNNEKKKNANKQKIELPPYDGKTTYGVLILDDGKQYSFNSGKPAPIYRNYIPASHVEGKAAIYMRENKIQSGTVYHNNTDGTCPYCDKMLPTLLEKDSTLKVVPPQNATSSKKGWITNEKIYIGNDKIPKTAR

SEQ ID NO: 10 - protein, 137 aa, Paraburkholderia guartelaensis
REDALLREQFPSMDAVTLPPFDGKTTIGYMFYTDANGQYHVRKLYSGGKVLSNYDSSGHVEGMAALIMRKGRITEAVVMHNHPSGTCHYCNGQVETLLPKNAKLKVIPPANAKAPTKYWYDQPVDYLGNSNDPKPPS

SEQ ID NO: 11 - protein, 136 aa, Coriobacteriia bacterium
SGFYQPANIPSSEVVLPEFDGKTTYGELRTPDGKSIPLQSGDPDPQYSNYVSSSHVEGKAAQYMRENGIEQATVYHNNANGTCGYCDKMLPTLLPDGSELTVIPPASAVPNNPQAVAAPKTYTGNSAVPKTNPRFK

SEQ ID NO: 12 - protein, 142 aa, Pseudoduganella violaceinigra
GTNAAGSSGKNVRMPRDYASELPEYDGKTTHGVLVTNEGKVIQLRSGGKEEPYTGYKAVSASHVEGKAAIWIRENGSSGGTVYHNNTTGTCGYCNSQVKALLPEGVELKIVPPTNAVAKNAQARAVPTINVGNGTQPGRKQK

SEQ ID NO: 13 - protein, 145 aa, Phosphitispora fastidiosa
SRPRTPEDESIAAAIAERGKMTPAPKGKTSASVEGNSTESGWTTGPRAAENTEAVMELSKKMGHDLQPNKLLDQGKPGRYHASHAEKQAAVAAPNKPIAVSAPMCDNCRRFFRALARYTGKPQTVAEPRAVWVFRPDGSVLVVPK

SEQ ID NO: 14 - protein, 141 aa, synthetic construct
TETFPLGSNQIPRPGLPAYDGKTSGVFYYLDGDGGMQSKVFTSGNGDPRYTNYRNNGHVAQKSALYMRENNISNATVYHNNTNGTCGYCNTMTATFLPEGATLTVVPPENAVANNSRAIDYVKTYTGTSNDPKISPRYKGN

SEQ ID NO: 15 - protein, 138 aa, synthetic construct
TETFPLGSNQIPRPGLPAYDGKTSGVFYYLDGDGGMQSKVFKSGNSSTPSYPQYKAQSASHVAGKAALYMRENGINEATVFHNNPNGTCGFCDRQVPALLPKGAKLTVVPPSNSVANNVRAIPVPKTYIGNSTVPKIK

SEQ ID NO: 16 - protein, 142 aa, synthetic construct
SQYPCKEEMSAGAGESGRKTISLPEYDGTTTHGVLVLDDGTQIGFKSGNSSTPSYPQYKAQSASHVAGKAALYMRENGINEATVFHNNPNGTCGFCDRQVPALLPKGAKLTVVPPSNSVANNVRAIPVPKTYIGNSTVPKIK

SEQ ID NO: 17 - protein, 140 aa, synthetic construct
TETFPLGSNQIPRPGLPAYDGKTSGVFYYLDGDGGMQSKVFTSGGVTEFPNYANAGHVAGQSALFMRSNGVGEGVVYHNNTNGTCGYCNTMTATFLPEGATLTVVPPENAVANNSRAIDYVKTYTGTSNDPKISPRYKGN

SEQ ID NO: 18 - protein, 134 aa, synthetic construct
TETFPLGSNQIPRPGLPAYDGKTSGVFYYLDGDGGMQSKVFTSGGVTEFPNYANAGHVAGQSALFMRSNGVGEGVVFHNNPNGTCGFCDRQVPALLPKGAKLTVVPPSNSVANNVRAIPVPKTYIGNSTVPKIK

SEQ ID NO: 19 - protein, 139 aa, synthetic construct
SQYPCKEEMSAGAGESGRKTISLPEYDGTTTHGVLVLDDGTQIGFTSGNGDPRYTNYRNNGHVAQKSALYMRENNISNATVFHNNPNGTCGFCDRQVPALLPKGAKLTVVPPSNSVANNVRAIPVPKTYIGNSTVPKIK

SEQ ID NO: 20 - protein, 140 aa, synthetic construct
TETFPLGSNQIPRPGLPAYDGKTSGVFYYLDGDGGMQSKVFTSGGVTEFPNYANAGHVAGQSALFMRSNGVGEGVVFHNNPEGTCGFCVSMTETLLPENAKLTVVPPENAVANNSRAIDYVKTYTGTSNDPKISPRYKGN

SEQ ID NO: 21 - protein, 134 aa, synthetic construct
TETFPLGSNQIPRPGLPAYDGKTSGVFYYLDGDGGMQSKVFTSGGVTEFPNYANAGHVAGQSALFMRSNGVGEGVVFHNNPEGTCGFCVSMTETLLPENAKLTVVPPSNSVANNVRAIPVPKTYIGNSTVPKIK

SEQ ID NO: 22 - protein, 139 aa, synthetic construct
SQYPCKEEMSAGAGESGRKTISLPEYDGTTTHGVLVLDDGTQIGFTSGNGDPRYTNYRNNGHVAQKSALYMRENNISNATVYHNNTNGTCGYCNTMTATFLPEGATLTVVPPSNSVANNVRAIPVPKTYIGNSTVPKIK

SEQ ID NO: 23 - DNA, 47 bp, Homo sapiens
  source 1..47: mol_type = genomic DNA
tctagcctagccgtttactcaatcctctgatcagggtgagcatcaaa

SEQ ID NO: 24 - DNA, 47 bp, Homo sapiens
  source 1..47: mol_type = genomic DNA
tttgatgctcaccctgatcagaggattgagtaaacggctaggctaga

SEQ ID NO: 25 - protein, 138 aa, synthetic construct
GSYALGPYQISAPQLPAYNGQTVGTFYYVNDAGGLESKVFSSGGPTPYPNYANAGHVAGQSALFMRDNGISEGLVFHNNPEGTCGFCVNMTETLLPENAKMTVVPPEGAIPVKRGATGETKVFTGNSNSPKSPTKGGC

SEQ ID NO: 26 - protein, 138 aa, synthetic construct
GSYTLGSYQISAPQLPAYNGQTVGTFYYVNDAGGLESRTFSSGGPTPYPNYANAGHVAGQSALFMRDNGISDGLVFHNNPEGTCGFCVNMTETLLPENSKLTVVPPEGSIPVKRGATGETRTFTGNSKSPKSPVKGGC

SEQ ID NO: 27 - protein, 140 aa, synthetic construct
TETFPLGSNQIPRPGLPAYDGKTSGVFYYLDGDGGMQSKVFTSGGVTEFPNYANAGHVAGQSALFMRSNGVGEGVVFHNNPEGTCGFCVSMTETLLPENAKLTVVPPEGAVPVKRGATGDVRVFTGNKNSPKSPSSGGAC

SEQ ID NO: 28 - protein, 145 aa, synthetic construct
SQYPCKEEMSAGAGESGRKTISLPEYDGTTTHGVLVLDDGTQIGFTSGNGDPRYTNYRNNGHVAQKSALYMRENNISNATVYHNNTNGTCGYCNTMTATFLPEGATLTVVPPENAVANNSRAIDYVKTYTGTSNDPKISPRYKGN

SEQ ID NO: 29 - protein, 125 aa, synthetic construct
GGSSSINLPEYDGKTTHGVLVLDDGTQVPFSSGNANPNYKNYIPASHVAGKSAIYMRENGINNGTVFHNNTDGTCPYCDKMLPTLLEEGSTLTVVPPANANAPKPSWVDTVKTYIGNDKIPKKPK

SEQ ID NO: 30 - protein, 146 aa, synthetic construct
QSNMSITDRLAKQKEKQDNTNIIDNRPKLPDYDGKTTHGILVTPNSEHIPFSSGNPNPNYKNYIPASHVAGKSAIYMRENGITSGTIYYNNTDGTCPYCDKMLSTLLEEGSVLEVIPPINAKAPKPSWVDKPKTYIGNNKVPKPNK

SEQ ID NO: 31 - protein, 130 aa, synthetic construct
NANCNQEKPVLPKYDGKTTEGVMVTPDGKQISFKSGNSSTPSYPQYKAQSASHVAGKAALYMRENGINEATVFHNNPNGTCGFCDRQVPALLPKGAKLTVVPPSNSVANNVRAIPVPKTYIGNSTVPKIK

SEQ ID NO: 32 - protein, 136 aa, synthetic construct
ALLREAYPSMEGATLPPFDGKTTIGLMFYTDASGQYQVKKLFSGEKVLSNYDATGHVAGKAALIMRNEKITEAVVMHNHPSGTCNYCDKQVETLLPKNATLRVIPPENAKAPTSYWNDQPTTYRGDGKDPKAPSKK

SEQ ID NO: 33 - DNA, 45 bp, Homo sapiens
  source 1..45: mol_type = genomic DNA
gctagtaaccacgttctcctgatcaaatatcactctcctacttac

SEQ ID NO: 34 - DNA, 45 bp, Homo sapiens
  source 1..45: mol_type = genomic DNA
gtaagtaggagagtgatatttgatcaggagaacgtggttactagc

SEQ ID NO: 35 - protein, 108 aa, Burkholderia cenocepacia
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 36 - protein, 29 aa, Burkholderia cenocepacia
AIPVKRGATGETKVFTGNSNSPKSPTKGG

SEQ ID NO: 37 - protein, 108 aa, Burkholderia gladioli
GSYTLGSYQISAPQLPAYNGQTVGTFYYVNDAGGLESRTFSSGGPTPYPNYANAGHVEGQSALFMRDNGISDGLVFHNNPEGTCGFCVNMTETLLPENSKLTVVPPEG

SEQ ID NO: 38 - protein, 30 aa, Burkholderia gladioli
SIPVKRGATGETRTFTGNSKSPKSPVKGGC

SEQ ID NO: 39 - protein, 109 aa, Caballeronia sp.
TETFPLGSNQIPRPGLPAYDGKTSGVFYYLDGDGGMQSKVFTSGGVTEFPNYANAGHVEGQSALFMRSNGVGEGVVFHNNPEGTCGFCVSMTETLLPENAKLTVVPPEG

SEQ ID NO: 40 - protein, 31 aa, Caballeronia sp.
AVPVKRGATGDVRVFTGNKNSPKSPSSGGAC

SEQ ID NO: 41 - protein, 114 aa, Roseburia intestinalis
SQYPCKEEMSAGAGESGRKTISLPEYDGTTTHGVLVLDDGTQIGFTSGNGDPRYTNYRNNGHVEQKSALYMRENNISNATVYHNNTNGTCGYCNTMTATFLPEGATLTVVPPEN

SEQ ID NO: 42 - protein, 31 aa, Roseburia intestinalis
AVANNSRAIDYVKTYTGTSNDPKISPRYKGN

SEQ ID NO: 43 - protein, 99 aa, Falcatimonas sp.
GGSSSINLPEYDGKTTHGVLVLDDGTQVPFSSGNANPNYKNYIPASHVEGKSAIYMRENGINNGTVFHNNTDGTCPYCDKMLPTLLEEGSTLTVVPPAN

SEQ ID NO: 44 - protein, 26 aa, Falcatimonas sp.
ANAPKPSWVDTVKTYIGNDKIPKKPK

SEQ ID NO: 45 - protein, 120 aa, Clostridium sp.
QSNMSITDRLAKQKEKQDNTNIIDNRPKLPDYDGKTTHGILVTPNSEHIPFSSGNPNPNYKNYIPASHVEGKSAIYMRENGITSGTIYYNNTDGTCPYCDKMLSTLLEEGSVLEVIPPIN

SEQ ID NO: 46 - protein, 26 aa, Clostridium sp.
AKAPKPSWVDKPKTYIGNNKVPKPNK

SEQ ID NO: 47 - protein, 105 aa, Rumiococcus bicirculans
NANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSN

SEQ ID NO: 48 - protein, 25 aa, Rumiococcus bicirculans
SVANNVRAIPVPKTYIGNSTVPKIK

SEQ ID NO: 49 - protein, 108 aa, Burkholderia ubonensis
ALLREAYPSMEGATLPPFDGKTTIGLMFYTDASGQYQVKKLFSGEKVLSNYDATGHVEGKAALIMRNEKITEAVVMHNHPSGTCNYCDKQVETLLPKNATLRVIPPEN

SEQ ID NO: 50 - protein, 28 aa, Burkholderia ubonensis
AKAPTSYWNDQPTTYRGDGKDPKAPSKK

SEQ ID NO: 51 - protein, 654 aa, synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQAHGLTPDQVVAIASNGGGKQALETVQRLLPVLCQDHGLTPAQVVAIASNIGGKQALETVQRLLPVLCQDHGLTPDQVVAIASNNGGKQALETVQRLLPVLCQAHGLTPDQVVAIASHDGGKQALETVQRLLPVLCQDHGLTPDQVVAIASHDGGKQALETVQRLLPVLCQAHGLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASHDGGKQALETVQRLLPVLCQDHGLTPDQVVAIASHDGGKQALETVQRLLPVLCQAHGLTPEQVVAIASNNGGKQALETVQRLLPVLCQAHGLTPAQVVAIASNGGGKQALETVQRLLPVLCQAHGLTPAQVVAIASNGGGKQALETVQRLLPVLCQAHGLTPEQVVAIASNGGGKQALESIVAQLSRPDPALAALLV

SEQ ID NO: 52 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 53 - protein, 166 aa, Escherichia coli
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 54 - DNA, 22 bp, synthetic construct
  source 1..22: mol_type = other DNA
actgactgcggccgcgtggtgt

SEQ ID NO: 55 - DNA, 35 bp, synthetic construct
  source 1..35: mol_type = other DNA
actgactggggcccttaacaaccgcccttcacagg

SEQ ID NO: 56 - DNA, 36 bp, synthetic construct
  source 1..36: mol_type = other DNA
actgactggggcccttagcacgcacctcctgaggat

SEQ ID NO: 57 - DNA, 42 bp, synthetic construct
  source 1..42: mol_type = other DNA
actgactggggcccttagttgcccttatatctggggctgatc

SEQ ID NO: 58 - DNA, 45 bp, synthetic construct
  source 1..45: mol_type = other DNA
actgactggggcccttatttaggctttttagggatcttgtcgttc

SEQ ID NO: 59 - DNA, 41 bp, synthetic construct
  source 1..41: mol_type = other DNA
actgactggggcccttacttgttaggcttaggcaccttgtt

SEQ ID NO: 60 - DNA, 41 bp, synthetic construct
  source 1..41: mol_type = other DNA
actgactggggcccttactttatctttggcaccgttgaatt

SEQ ID NO: 61 - DNA, 38 bp, synthetic construct
  source 1..38: mol_type = other DNA
actgactggggcccttatttcttgctaggggccttggg

SEQ ID NO: 62 - DNA, 31 bp, synthetic construct
  source 1..31: mol_type = other DNA
cgtggtctagaggatcttacacactgggctc

SEQ ID NO: 63 - DNA, 24 bp, synthetic construct
  source 1..24: mol_type = other DNA
gcctcggatccgccagaacaaccg

SEQ ID NO: 64 - DNA, 33 bp, synthetic construct
  source 1..33: mol_type = other DNA
cgtggtctagaagcatccccgtgaaaagaggcg

SEQ ID NO: 65 - DNA, 24 bp, synthetic construct
  source 1..24: mol_type = other DNA
gcctcggatccgccagaacaaccg

SEQ ID NO: 66 - DNA, 30 bp, synthetic construct
  source 1..30: mol_type = other DNA
cgtggtctagaactgagaccttcccgctcg

SEQ ID NO: 67 - DNA, 37 bp, synthetic construct
  source 1..37: mol_type = other DNA
gcctcggatccgccagaaccttctggaggcactaccg

SEQ ID NO: 68 - DNA, 28 bp, synthetic construct
  source 1..28: mol_type = other DNA
cgtggtctagagcagtgcccgttaagcg

SEQ ID NO: 69 - DNA, 35 bp, synthetic construct
  source 1..35: mol_type = other DNA
gcctcggatccgccagagcacgcacctcctgagga

SEQ ID NO: 70 - DNA, 29 bp, synthetic construct
  source 1..29: mol_type = other DNA
cgtggtctagaagccagtacccctgcaag

SEQ ID NO: 71 - DNA, 36 bp, synthetic construct
  source 1..36: mol_type = other DNA
gcctcggatccgccagagttctcaggaggcaccacg

SEQ ID NO: 72 - DNA, 28 bp, synthetic construct
  source 1..28: mol_type = other DNA
cgtggtctagagccgtggccaacaacag

SEQ ID NO: 73 - DNA, 23 bp, synthetic construct
  source 1..23: mol_type = other DNA
gcctcggatccgccagagttgcc

SEQ ID NO: 74 - DNA, 36 bp, synthetic construct
  source 1..36: mol_type = other DNA
cgtggtctagaggcggttcttctagtatcaacctgc

SEQ ID NO: 75 - DNA, 37 bp, synthetic construct
  source 1..37: mol_type = other DNA
gcctcggatccgccagaattcgctggtggcactacgg

SEQ ID NO: 76 - DNA, 31 bp, synthetic construct
  source 1..31: mol_type = other DNA
cgtggtctagagcaaacgcaccaaaacccag

SEQ ID NO: 77 - DNA, 45 bp, synthetic construct
  source 1..45: mol_type = other DNA
gcctcggatccgccagatttaggctttttagggatcttgtcgttc

SEQ ID NO: 78 - DNA, 31 bp, synthetic construct
  source 1..31: mol_type = other DNA
cgtggtctagacagagcaacatgagcatcac

SEQ ID NO: 79 - DNA, 41 bp, synthetic construct
  source 1..41: mol_type = other DNA
ggctcggatccgccagagttgataggtgggatcacctccag

SEQ ID NO: 80 - DNA, 29 bp, synthetic construct
  source 1..29: mol_type = other DNA
cgtggtctagagccaaggcccctaaaccc

SEQ ID NO: 81 - DNA, 27 bp, synthetic construct
  source 1..27: mol_type = other DNA
gcctcggatccgccagacttgttaggc

SEQ ID NO: 82 - DNA, 38 bp, synthetic construct
  source 1..38: mol_type = other DNA
cgtggtctagaaatgctaattgcaatcaagagaagccc

SEQ ID NO: 83 - DNA, 37 bp, synthetic construct
  source 1..37: mol_type = other DNA
gcctcggatccgccagagttactgggtggaacgacgg

SEQ ID NO: 84 - DNA, 35 bp, synthetic construct
  source 1..35: mol_type = other DNA
cgtggtctagatcagttgccaacaatgtaagagcg

SEQ ID NO: 85 - DNA, 43 bp, synthetic construct
  source 1..43: mol_type = other DNA
gcctcggatccgccagactttatctttggcaccgttgaattac

SEQ ID NO: 86 - DNA, 27 bp, synthetic construct
  source 1..27: mol_type = other DNA
cgtggtctagagctctgctgagagagg

SEQ ID NO: 87 - DNA, 35 bp, synthetic construct
  source 1..35: mol_type = other DNA
gcctcggatccgccagagttctcgggggggatcac

SEQ ID NO: 88 - DNA, 28 bp, synthetic construct
  source 1..28: mol_type = other DNA
cgtggtctagagccaaggccccaacaag

SEQ ID NO: 89 - DNA, 28 bp, synthetic construct
  source 1..28: mol_type = other DNA
gcctcggatccgccagatttcttgctag

SEQ ID NO: 90 - DNA, 17 bp, synthetic construct
  source 1..17: mol_type = other DNA
ccccaacgttgtaggcc

SEQ ID NO: 91 - DNA, 21 bp, synthetic construct
  source 1..21: mol_type = other DNA
ggttggtctctgctagtgtgg

SEQ ID NO: 92 - DNA, 19 bp, synthetic construct
  source 1..19: mol_type = other DNA
caccggcgcagtcattctc

SEQ ID NO: 93 - DNA, 21 bp, synthetic construct
  source 1..21: mol_type = other DNA
catgggggcatgagttagcag

SEQ ID NO: 94 - protein, 30 aa, synthetic construct
MASVLTPLLLRGLTGSARRLPVPRAKIHSL

SEQ ID NO: 95 - protein, 31 aa, synthetic construct
MALSRAVCGTSRQLAPVLGYLGSRQKHSLPD

SEQ ID NO: 96 - protein, 16 aa, Homo sapiens
SGSETPGTSESATPES

SEQ ID NO: 97 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 98 - protein, 133 aa, synthetic construct
SNNEKKKNANKQKIELPPYDGKTTYGVLILDDGKQYSFNSGKPAPIYRNYIPASHVAGKAAIYMRENKIQSGTVYHNNTDGTCPYCDKMLPTLLEKDSTLKVVPPQNATSSKKGWITNEKIYIGNDKIPKTAR

SEQ ID NO: 99 - protein, 137 aa, synthetic construct
REDALLREQFPSMDAVTLPPFDGKTTIGYMFYTDANGQYHVRKLYSGGKVLSNYDSSGHVAGMAALIMRKGRITEAVVMHNHPSGTCHYCNGQVETLLPKNAKLKVIPPANAKAPTKYWYDQPVDYLGNSNDPKPPS

SEQ ID NO: 100 - protein, 136 aa, synthetic construct
SGFYQPANIPSSEVVLPEFDGKTTYGELRTPDGKSIPLQSGDPDPQYSNYVSSSHVAGKAAQYMRENGIEQATVYHNNANGTCGYCDKMLPTLLPDGSELTVIPPASAVPNNPQAVAAPKTYTGNSAVPKTNPRFK

SEQ ID NO: 101 - protein, 142 aa, synthetic construct
GTNAAGSSGKNVRMPRDYASELPEYDGKTTHGVLVTNEGKVIQLRSGGKEEPYTGYKAVSASHVAGKAAIWIRENGSSGGTVYHNNTTGTCGYCNSQVKALLPEGVELKIVPPTNAVAKNAQARAVPTINVGNGTQPGRKQK

SEQ ID NO: 102 - protein, 145 aa, synthetic construct
SRPRTPEDESIAAAIAERGKMTPAPKGKTSASVEGNSTESGWTTGPRAAENTEAVMELSKKMGHDLQPNKLLDQGKPGRYHASHAAKQAAVAAPNKPIAVSAPMCDNCRRFFRALARYTGKPQTVAEPRAVWVFRPDGSVLVVPK

SEQ ID NO: 103 - protein, 107 aa, Treponema sp.
SNNEKKKNANKQKIELPPYDGKTTYGVLILDDGKQYSFNSGKPAPIYRNYIPASHVEGKAAIYMRENKIQSGTVYHNNTDGTCPYCDKMLPTLLEKDSTLKVVPPQN

SEQ ID NO: 104 - protein, 26 aa, Treponema sp.
ATSSKKGWITNEKIYIGNDKIPKTAR

SEQ ID NO: 105 - protein, 111 aa, Paraburkholderia guartelaensis
REDALLREQFPSMDAVTLPPFDGKTTIGYMFYTDANGQYHVRKLYSGGKVLSNYDSSGHVEGMAALIMRKGRITEAVVMHNHPSGTCHYCNGQVETLLPKNAKLKVIPPAN

SEQ ID NO: 106 - protein, 26 aa, Paraburkholderia guartelaensis
AKAPTKYWYDQPVDYLGNSNDPKPPS

SEQ ID NO: 107 - protein, 107 aa, Coriobacteriia bacterium
SGFYQPANIPSSEVVLPEFDGKTTYGELRTPDGKSIPLQSGDPDPQYSNYVSSSHVEGKAAQYMRENGIEQATVYHNNANGTCGYCDKMLPTLLPDGSELTVIPPAS

SEQ ID NO: 108 - protein, 29 aa, Coriobacteriia bacterium
AVPNNPQAVAAPKTYTGNSAVPKTNPRFK

SEQ ID NO: 109 - protein, 115 aa, Pseudoduganella violaceinigra
GTNAAGSSGKNVRMPRDYASELPEYDGKTTHGVLVTNEGKVIQLRSGGKEEPYTGYKAVSASHVEGKAAIWIRENGSSGGTVYHNNTTGTCGYCNSQVKALLPEGVELKIVPPTN

SEQ ID NO: 110 - protein, 27 aa, Pseudoduganella violaceinigra
AVAKNAQARAVPTINVGNGTQPGRKQK

SEQ ID NO: 111 - protein, 124 aa, Phosphitispora fastidiosa
SRPRTPEDESIAAAIAERGKMTPAPKGKTSASVEGNSTESGWTTGPRAAENTEAVMELSKKMGHDLQPNKLLDQGKPGRYHASHAEKQAAVAAPNKPIAVSAPMCDNCRRFFRALARYTGKPQT

SEQ ID NO: 112 - protein, 21 aa, Phosphitispora fastidiosa
VAEPRAVWVFRPDGSVLVVPK

SEQ ID NO: 113 - DNA, 18 bp, synthetic construct
  source 1..18: mol_type = other DNA
taccctcccttagcaggg

SEQ ID NO: 114 - DNA, 22 bp, synthetic construct
  source 1..22: mol_type = other DNA
ctcgtgtgtctacgtctattcc

SEQ ID NO: 115 - DNA, 47 bp, Homo sapiens
  source 1..47: mol_type = genomic DNA
tgctagtaaccacgttctcctgatcaaatatcactctcctacttaca

SEQ ID NO: 116 - DNA, 47 bp, Homo sapiens
  source 1..47: mol_type = genomic DNA
tgtaagtaggagagtgatatttgatcaggagaacgtggttactagca

SEQ ID NO: 117 - DNA, 45 bp, Homo sapiens
  source 1..45: mol_type = genomic DNA
ctagcctagccgtttactcaatcctctgatcagggtgagcatcaa

SEQ ID NO: 118 - DNA, 45 bp, Homo sapiens
  source 1..45: mol_type = genomic DNA
ttgatgctcaccctgtacagaggattgagtaaacggctaggctag

SEQ ID NO: 119 - DNA, 45 bp, Homo sapiens; identical to SEQ ID NO: 117
  source 1..45: mol_type = genomic DNA

SEQ ID NO: 120 - DNA, 45 bp, Homo sapiens
  source 1..45: mol_type = genomic DNA
ttgatgctcaccctgatcagaggattgagtaaacggctaggctag

SEQ ID NO: 121 - DNA, 46 bp, Homo sapiens
  source 1..46: mol_type = genomic DNA
tcttcgaccccgccggaggaggagaccccattctataccaacacct

SEQ ID NO: 122 - DNA, 46 bp, Homo sapiens
  source 1..46: mol_type = genomic DNA
aggtattggtatagaatggggtctcctcctccggcggggtcgaaga

SEQ ID NO: 123 - DNA, 32 bp, Homo sapiens
  source 1..32: mol_type = genomic DNA
gctagtaaccacggaattgactctccccctcc

SEQ ID NO: 124 - DNA, 32 bp, Homo sapiens
  source 1..32: mol_type = genomic DNA
ggagggggagagtcaattccgtggttactagc

(19) United States
(12) Patent Application Publication
(10) Pub. No.: US 2026/0193629 A1
(43) Pub. Date: Jul. 9, 2026

(54) CHIMERIC POLYPEPTIDES AND THEIR USE FOR EDITING MITOCHONDRIAL AND GENOMIC DNA

(71) Applicant: Mayo Foundation for Medical Education and Research, Rochester, MN (US)

(72) Inventors: Stephen C. Ekker, Austin, TX (US); Karl J. Clark, Rochester, MN (US)

(21) Appl. No.: 19/129,749

(22) PCT Filed: Sep. 14, 2023
(86) PCT No.: PCT/US2023/032702

§ 371 Date: May 14, 2025

Related U.S. Application Data
(60) Provisional application No. 63/425,663, filed on Nov. 15, 2022.

(51) Int. Cl. C12N 9/22; C12N 9/78

(52) U.S. Cl. CPC C12N 9/226; C12N 9/78; C12Y 305/04001; C07K 2319/00

(57) ABSTRACT

Methods and materials for editing DNA (e.g., mitochondrial DNA) are provided herein. For example, chimeric polypeptides containing a nucleic acid interacting domain, a facilitating domain, and a base editing domain are provided herein, as are methods for using chimeric polypeptides to edit mitochondrial or nuclear DNA.

### CROSS-REFERENCE TO RELATED APPLICATIONS

This application claims priority from U.S. Provisional Application Ser. No. 63/425,663, filed Nov. 15, 2022. The disclosure of the prior application is considered part of (and is incorporated by reference in) the disclosure of this application.

### STATEMENT AS TO FEDERALLY SPONSORED RESEARCH

This invention was made with government support under AI142773 awarded by the National Institutes of Health. The government has certain rights in the invention.

### SEQUENCE LISTING

This application contains a Sequence Listing that has been submitted electronically as an XML file named “07039-2175WO1 SL ST26.XML.” The XML file, created on Sep. 13, 2023, is 118,517 bytes in size. The material in the XML file is hereby incorporated by reference in its entirety.

### TECHNICAL FIELD

This document relates to methods and materials for transiently enabling nucleic acid to be modified, such as by generation of single base edits in DNA. For example, this document provides chimeric polypeptides that can be used to generate single base edits in mitochondrial and/or nuclear genomic DNA.

### BACKGROUND

Human mitochondrial diseases are commonly caused by mutations in the mitochondrial DNA (mtDNA), or by mutations within the nuclear genome that are involved in mitochondrial function. Significant progress in attempts to correct such mutations has been made using CRISPR-based techniques in nuclear gene editing. However, gene editing of the mitochondrial genome using the CRISPR system has been challenging, mainly due to sub-efficient delivery of guide RNA into the mitochondria.

### SUMMARY

This document provides methods and materials for transiently enabling DNA to be subsequently modified, such as through base editing. For example, this document provides chimeric polypeptides that include a first portion containing a nucleic acid interacting domain, a second portion that includes a facilitating domain, and a third portion that includes a base editing (e.g., a deaminase) domain, where the base editing domain is extrinsic to the polypeptide from which the facilitating domain is derived. In another example, this document provides chimeric polypeptides that include a first portion containing a nucleic acid interacting domain, and a second portion that includes a facilitating domain and a base editing (e.g., deaminase) domain or a portion thereof, such that the base editing domain or portion thereof is intrinsic to the polypeptide from which the facilitating domain was derived. This document also provides methods and materials for using the chimeric polypeptides provided herein to generate specific base changes (e.g., single base C-to-T changes or single base G-to-A changes on the opposite strand generated by cytosine base editors; or single base A-to-G changes or T-to-C changes on the opposite strand generated by adenine base editors) in mitochondrial and/or nuclear DNA.

As described herein, chimeric polypeptides can be designed to have the ability to bind dsDNA and facilitate and carry out subsequent and separable programmable DNA modifications such as single base editing. In general, the chimeric polypeptides described herein can contain a nucleic acid interacting domain, a facilitating domain, and a base editing domain. Without being bound by any particular mechanism of action, the chimeric polypeptides described herein can function by interacting with a particular target nucleic acid sequence of dsDNA and transiently separate the dsDNA strands in that location into single-stranded DNA to form an effective substrate for the base editing domain to carry out single base editing. In such cases, the function of the facilitating domain of the chimeric polypeptide can be referred to as a “modular DNA meltase” or a “meltase” for short. The primary functional readout in the studies described herein is net DNA base editing due to a two-step process: a DNA-interaction step that facilitates the editing (in some cases, a “meltase” reaction), and a subsequent editing step by the tethered base editor. As demonstrated herein, the base editor can be an extrinsic deaminase (e.g., an extrinsic adenosine deaminase), or the base editor can be an intrinsic cytosine deaminase.

In general, one aspect of this document features a chimeric polypeptide containing a nucleic acid interacting domain and a facilitating domain, where the facilitating domain includes (or consists essentially of or consists of):
an amino acid sequence (i) that includes the amino acid sequence set forth in SEQ ID NO: 2, (ii) that is at least 92 percent identical to the amino acid sequence set forth in SEQ ID NO:2, (iii) that includes an N-terminal portion of the amino acid sequence set forth in SEQ ID NO:2 that is at least 100 amino acid residues in length, (iv) that is at least 92 percent identical to the N-terminal portion, (v) that includes a C-terminal portion of the amino acid sequence set forth in SEQ ID NO:2 that is at least 25 amino acid residues in length, or (vi) that is at least 92 percent identical to the C-terminal portion;
an amino acid sequence (i) that includes the amino acid sequence set forth in SEQ ID NO: 3, (ii) that is at least 85 percent identical to the amino acid sequence set forth in SEQ ID NO:3, (iii) that includes an N-terminal portion of the amino acid sequence set forth in SEQ ID NO:3 that is at least 100 amino acid residues in length, (iv) that is at least 85 percent identical to the N-terminal portion, (v) that includes a C-terminal portion of the amino acid sequence set forth in SEQ ID NO:3 that is at least 25 amino acid residues in length, or (vi) that is at least 85 percent identical to the C-terminal portion;
an amino acid sequence (i) that includes the amino acid sequence set forth in SEQ ID NO: 4, (ii) that is at least 85 percent identical to the amino acid sequence set forth in SEQ ID NO:4, (iii) that includes an N-terminal portion of the amino acid sequence set forth in SEQ ID NO:4 that is at least 105 amino acid residues in length, (iv) that is at least 85 percent identical to the N-terminal portion, (v) that includes a C-terminal portion of the amino acid sequence set forth in SEQ ID NO:4 that is at least 25 amino acid residues in length, or (vi) that is at least 85 percent identical to the C-terminal portion;
an amino acid sequence (i) that includes the amino acid sequence set forth in SEQ ID NO: 5, (ii) that is at least 85 percent identical to the amino acid sequence set forth in SEQ ID NO:5, (iii) that includes an N-terminal portion of the amino acid sequence set forth in SEQ ID NO:5 that is at least 90 amino acid residues in length, (iv) that is at least 85 percent identical to the N-terminal portion, (v) that includes a C-terminal portion of the amino acid sequence set forth in SEQ ID NO:5 that is at least 20 amino acid residues in length, or (vi) that is at least 85 percent identical to the C-terminal portion;
an amino acid sequence (i) that includes the amino acid sequence set forth in SEQ ID NO: 6, (ii) that is at least 85 percent identical to the amino acid sequence set forth in SEQ ID NO:6, (iii) that includes an N-terminal portion of the amino acid sequence set forth in SEQ ID NO:6 that is at least 110 amino acid residues in length, (iv) that is at least 85 percent identical to the N-terminal portion, (v) that includes a C-terminal portion of the amino acid sequence set forth in SEQ ID NO:6 that is at least 20 amino acid residues in length, or (vi) that is at least 85 percent identical to the C-terminal portion;
an amino acid sequence (i) that includes the amino acid sequence set forth in SEQ ID NO: 7, (ii) that is at least 85 percent identical to the amino acid sequence set forth in SEQ ID NO:7, (iii) that includes an N-terminal portion of the amino acid sequence set forth in SEQ ID NO:7 that is at least 100 amino acid residues in length, (iv) that is at least 85 percent identical to the N-terminal portion, (v) that includes a C-terminal portion of the amino acid sequence set forth in SEQ ID NO:7 that is at least 20 amino acid residues in length, or (vi) that is at least 85 percent identical to the C-terminal portion;
an amino acid sequence (i) that includes the amino acid sequence set forth in SEQ ID NO: 8, (ii) that is at least 85 percent identical to the amino acid sequence set forth in SEQ ID NO:8, (iii) that includes an N-terminal portion of the amino acid sequence set forth in SEQ ID NO:8 that is at least 100 amino acid residues in length, (iv) that is at least 85 percent identical to the N-terminal portion, (v) that includes a C-terminal portion of the amino acid sequence set forth in SEQ ID NO:8 that is at least 20 amino acid residues in length, or (vi) that is at least 85 percent identical to the C-terminal portion;
an amino acid sequence (i) that comprises the amino acid sequence set forth in SEQ ID NO:9, (ii) that is at least 90 percent identical to the amino acid sequence set forth in SEQ ID NO:9, (iii) that comprises an N-terminal portion of the amino acid sequence set forth in SEQ ID NO:9 that is at least 100 amino acid residues in length, (iv) that is at least 93% identical to said N-terminal portion, (v) that comprises a C-terminal portion of the amino acid sequence set forth in SEQ ID NO:9 that is at least 23 amino acid residues in length, or (vi) that is at least 88% identical to said C-terminal portion;
an amino acid sequence (i) that comprises the amino acid sequence set forth in SEQ ID NO:10, (ii) that is at least 90 percent identical to the amino acid sequence set forth in SEQ ID NO:10, (iii) that comprises an N-terminal portion of the amino acid sequence set forth in SEQ ID NO: 10 that is at least 105 amino acid residues in length, (iv) that is at least 94% identical to said N-terminal portion, (v) that comprises a C-terminal portion of the amino acid sequence set forth in SEQ ID NO:10 that is at least 23 amino acid residues in length, or (vi) that is at least 88% identical to said C-terminal portion;
an amino acid sequence (i) that comprises the amino acid sequence set forth in SEQ ID NO:11, (ii) that is at least 90 percent identical to the amino acid sequence set forth in SEQ ID NO:11, (iii) that comprises an N-terminal portion of the amino acid sequence set forth in SEQ ID NO:11 that is at least 100 amino acid residues in length, (iv) that is at least 93% identical to said N-terminal portion, (v) that comprises a C-terminal portion of the amino acid sequence set forth in SEQ ID NO:11 that is at least 25 amino acid residues in length, or (vi) that is at least 86% identical to said C-terminal portion;
an amino acid sequence (i) that comprises the amino acid sequence set forth in SEQ ID NO:12, (ii) that is at least 90 percent identical to the amino acid sequence set forth in SEQ ID NO: 12, (iii) that comprises an N-terminal portion of the amino acid sequence set forth in SEQ ID NO: 12 that is at least 108 amino acid residues in length, (iv) that is at least 93% identical to said N-terminal portion, (v) that comprises a C-terminal portion of the amino acid sequence set forth in SEQ ID NO:12 that is at least 23 amino acid residues in length, or (vi) that is at least 85% identical to said C-terminal portion; or
an amino acid sequence (i) that comprises the amino acid sequence set forth in SEQ ID NO:13, (ii) that is at least 90 percent identical to the amino acid sequence set forth in SEQ ID NO:13, (iii) that comprises an N-terminal portion of the amino acid sequence set forth in SEQ ID NO: 13 that is at least 116 amino acid residues in length, (iv) that is at least 93% identical to said N-terminal portion, (v) that comprises a C-terminal portion of the amino acid sequence set forth in SEQ ID NO:13 that is at least 18 amino acid residues in length, or (vi) that is at least 85% identical to said C-terminal portion.

The nucleic acid interacting domain can be N-terminal to the facilitating domain. The nucleic acid interacting domain can include a transcription activator-like effector (TALE) DNA binding domain. The nucleic acid interacting domain can include a zinc finger DNA binding domain. The nucleic acid interacting domain can include CRISPR/Cas DNA binding components. The chimeric polypeptide can further include a linker between the nucleic acid interacting domain and the facilitating domain. The chimeric polypeptide can further include a mitochondrial targeting sequence (MTS). The MTS can be an isocitrate dehydrogenase 2 MTS, a human COX8A MTS, or a human SOD2 MTS. The MTS can be at the N-terminus of the chimeric polypeptide. The chimeric polypeptide can include in order from N-terminus to C-terminus, the MTS, the nucleic acid interacting domain, and the facilitating domain, and the chimeric polypeptide can further include a linker between the MTS and the nucleic acid interacting domain. The facilitating domain can have intrinsic cytosine deaminase activity. The facilitating domain can lack cytosine deaminase activity, and the chimeric polypeptide can further include an extrinsic deaminase domain. The extrinsic deaminase domain can be an adenosine deaminase domain. The adenosine deaminase domain can have an amino acid sequence at least 85 percent identical to the amino acid sequence set forth in SEQ ID NO: 53. The extrinsic deaminase domain can be a cytosine deaminase domain. The chimeric polypeptide can include, in order from N-terminus to C-terminus, the nucleic acid interacting domain, the facilitating domain, and the extrinsic deaminase domain. The chimeric polypeptide can further include a MTS at the N-terminus of the chimeric polypeptide, a first linker between the nucleic acid interacting domain and the facilitating domain, and a second linker between the facilitating domain and the extrinsic deaminase domain.

In some cases, the facilitating domain can include the N-terminal portion of the amino acid sequence set forth in SEQ ID NO:2 that is at least 100 amino acid residues in length, or the amino acid sequence that is at least 92 percent identical to the N-terminal portion; the N-terminal portion of the amino acid sequence set forth in SEQ ID NO:3 that is at least 100 amino acid residues in length, or the amino acid sequence that is at least 85 percent identical to the N-terminal portion; the N-terminal portion of the amino acid sequence set forth in SEQ ID NO:4 that is at least 105 amino acid residues in length, or the amino acid sequence that is at least 85 percent identical to the N-terminal portion; the N-terminal portion of the amino acid sequence set forth in SEQ ID NO:5 that is at least 90 amino acid residues in length, or the amino acid sequence that is at least 85 percent identical to the N-terminal portion; the N-terminal portion of the amino acid sequence set forth in SEQ ID NO:6 that is at least 110 amino acid residues in length, or the amino acid sequence that is at least 85 percent identical to the N-terminal portion; the N-terminal portion of the amino acid sequence set forth in SEQ ID NO:7 that is at least 100 amino acid residues in length, or the amino acid sequence that is at least 85 percent identical to the N-terminal portion; or the N-terminal portion of the amino acid sequence set forth in SEQ ID NO:8 that is at least 100 amino acid residues in length, or the amino acid sequence that is at least 85 percent identical to the N-terminal portion.

In some cases, the facilitating domain can include the C-terminal portion of the amino acid sequence set forth in SEQ ID NO:2 that is at least 25 amino acid residues in length, or the amino acid sequence that is at least 92 percent identical to the C-terminal portion; the C-terminal portion of the amino acid sequence set forth in SEQ ID NO:3 that is at least 25 amino acid residues in length, or the amino acid sequence that is at least 85 percent identical to the C-terminal portion; the C-terminal portion of the amino acid sequence set forth in SEQ ID NO:4 that is at least 25 amino acid residues in length, or the amino acid sequence that is at least 85 percent identical to the C-terminal portion; the C-terminal portion of the amino acid sequence set forth in SEQ ID NO:5 that is at least 20 amino acid residues in length, or the amino acid sequence that is at least 85 percent identical to the C-terminal portion; the C-terminal portion of the amino acid sequence set forth in SEQ ID NO:6 that is at least 20 amino acid residues in length, or the amino acid sequence that is at least 85 percent identical to the C-terminal portion; the C-terminal portion of the amino acid sequence set forth in SEQ ID NO:7 that is at least 20 amino acid residues in length, or the amino acid sequence that is at least 85 percent identical to the C-terminal portion; or the C-terminal portion of the amino acid sequence set forth in SEQ ID NO:8 that is at least 20 amino acid residues in length, or the amino acid sequence that is at least 85 percent identical to the C-terminal portion.

In another aspect, this document features a nucleic acid that includes a nucleotide sequence encoding a chimeric polypeptide described herein. This document also features a vector containing the nucleic acid provided herein. In addition, this document features a cell containing the nucleic acid or the vector provided herein.

In another aspect, this document features a method for generating a mutation within mitochondrial or nuclear genomic DNA of a cell. The method can include (or consist essentially of or consist of) (a) introducing, into the cell, a chimeric polypeptide that includes a nucleic acid interacting domain and a facilitating domain, and (b) incubating the cell such that the chimeric polypeptide generates a mutation within the mitochondrial or the nucleic genomic DNA, wherein the facilitating domain includes:
(1) an amino acid sequence (i) that includes the amino acid sequence set forth in SEQ ID NO:2, (ii) that is at least 92 percent identical to the amino acid sequence set forth in SEQ ID NO:2, (iii) that includes an N-terminal portion of the amino acid sequence set forth in SEQ ID NO:2 that is at least 100 amino acid residues in length, (iv) that is at least 92 percent identical to the N-terminal portion, (v) that includes a C-terminal portion of the amino acid sequence set forth in SEQ ID NO:2 that is at least 25 amino acid residues in length, or (vi) that is at least 92 percent identical to the C-terminal portion;
(2) an amino acid sequence (i) that includes the amino acid sequence set forth in SEQ ID NO:3, (ii) that is at least 85 percent identical to the amino acid sequence set forth in SEQ ID NO:3, (iii) that includes an N-terminal portion of the amino acid sequence set forth in SEQ ID NO:3 that is at least 100 amino acid residues in length, (iv) that is at least 85 percent identical to the N-terminal portion, (v) that includes a C-terminal portion of the amino acid sequence set forth in SEQ ID NO:3 that is at least 25 amino acid residues in length, or (vi) that is at least 85 percent identical to the C-terminal portion;
(3) an amino acid sequence (i) that includes the amino acid sequence set forth in SEQ ID NO:4, (ii) that is at least 85 percent identical to the amino acid sequence set forth in SEQ ID NO:4, (iii) that includes an N-terminal portion of the amino acid sequence set forth in SEQ ID NO:4 that is at least 105 amino acid residues in length, (iv) that is at least 85 percent identical to the N-terminal portion, (v) that includes a C-terminal portion of the amino acid sequence set forth in SEQ ID NO:4 that is at least 25 amino acid residues in length, or (vi) that is at least 85 percent identical to the C-terminal portion;
(4) an amino acid sequence (i) that includes the amino acid sequence set forth in SEQ ID NO:5, (ii) that is at least 85 percent identical to the amino acid sequence set forth in SEQ ID NO:5, (iii) that includes an N-terminal portion of the amino acid sequence set forth in SEQ ID NO:5 that is at least 90 amino acid residues in length, (iv) that is at least 85 percent identical to the N-terminal portion, (v) that includes a C-terminal portion of the amino acid sequence set forth in SEQ ID NO:5 that is at least 20 amino acid residues in length, or (vi) that is at least 85 percent identical to the C-terminal portion;
(5) an amino acid sequence (i) that includes the amino acid sequence set forth in SEQ ID NO:6, (ii) that is at least 85 percent identical to the amino acid sequence set forth in SEQ ID NO:6, (iii) that includes an N-terminal portion of the amino acid sequence set forth in SEQ ID NO:6 that is at least 110 amino acid residues in length, (iv) that is at least 85 percent identical to the N-terminal portion, (v) that includes a C-terminal portion of the amino acid sequence set forth in SEQ ID NO:6 that is at least 20 amino acid residues in length, or (vi) that is at least 85 percent identical to the C-terminal portion;
(6) an amino acid sequence (i) that includes the amino acid sequence set forth in SEQ ID NO:7, (ii) that is at least 85 percent identical to the amino acid sequence set forth in SEQ ID NO:7, (iii) that includes an N-terminal portion of the amino acid sequence set forth in SEQ ID NO:7 that is at least 100 amino acid residues in length, (iv) that is at least 85 percent identical to the N-terminal portion, (v) that includes a C-terminal portion of the amino acid sequence set forth in SEQ ID NO:7 that is at least 20 amino acid residues in length, or (vi) that is at least 85 percent identical to the C-terminal portion; or
(7) an amino acid sequence (i) that includes the amino acid sequence set forth in SEQ ID NO:8, (ii) that is at least 85 percent identical to the amino acid sequence set forth in SEQ ID NO:8, (iii) that includes an N-terminal portion of the amino acid sequence set forth in SEQ ID NO:8 that is at least 100 amino acid residues in length, (iv) that is at least 85 percent identical to the N-terminal portion, (v) that includes a C-terminal portion of the amino acid sequence set forth in SEQ ID NO:8 that is at least 20 amino acid residues in length, or (vi) that is at least 85 percent identical to the C-terminal portion;
(8) an amino acid sequence (i) that comprises the amino acid sequence set forth in SEQ ID NO:9, (ii) that is at least 90 percent identical to the amino acid sequence set forth in SEQ ID NO:9, (iii) that comprises an N-terminal portion of the amino acid sequence set forth in SEQ ID NO:9 that is at least 100 amino acid residues in length, (iv) that is at least 93% identical to said N-terminal portion, (v) that comprises a C-terminal portion of the amino acid sequence set forth in SEQ ID NO:9 that is at least 23 amino acid residues in length, or (vi) that is at least 88% identical to said C-terminal portion;
(9) an amino acid sequence (i) that comprises the amino acid sequence set forth in SEQ ID NO:10, (ii) that is at least 90 percent identical to the amino acid sequence set forth in SEQ ID NO:10, (iii) that comprises an N-terminal portion of the amino acid sequence set forth in SEQ ID NO:10 that is at least 105 amino acid residues in length, (iv) that is at least 94% identical to said N-terminal portion, (v) that comprises a C-terminal portion of the amino acid sequence set forth in SEQ ID NO:10 that is at least 23 amino acid residues in length, or (vi) that is at least 88% identical to said C-terminal portion;
(10) an amino acid sequence (i) that comprises the amino acid sequence set forth in SEQ ID NO:11, (ii) that is at least 90 percent identical to the amino acid sequence set forth in SEQ ID NO:11, (iii) that comprises an N-terminal portion of the amino acid sequence set forth in SEQ ID NO: 11 that is at least 100 amino acid residues in length, (iv) that is at least 93% identical to said N-terminal portion, (v) that comprises a C-terminal portion of the amino acid sequence set forth in SEQ ID NO:11 that is at least 25 amino acid residues in length, or (vi) that is at least 86% identical to said C-terminal portion;
(11) an amino acid sequence (i) that comprises the amino acid sequence set forth in SEQ ID NO:12, (ii) that is at least 90 percent identical to the amino acid sequence set forth in SEQ ID NO:12, (iii) that comprises an N-terminal portion of the amino acid sequence set forth in SEQ ID NO:12 that is at least 108 amino acid residues in length, (iv) that is at least 93% identical to said N-terminal portion, (v) that comprises a C-terminal portion of the amino acid sequence set forth in SEQ ID NO: 12 that is at least 23 amino acid residues in length, or (vi) that is at least 85% identical to said C-terminal portion; or
(12) an amino acid sequence (i) that comprises the amino acid sequence set forth in SEQ ID NO: 13, (ii) that is at least 90 percent identical to the amino acid sequence set forth in SEQ ID NO:13, (iii) that comprises an N-terminal portion of the amino acid sequence set forth in SEQ ID NO: 13 that is at least 116 amino acid residues in length, (iv) that is at least 93% identical to said N-terminal portion, (v) that comprises a C-terminal portion of the amino acid sequence set forth in SEQ ID NO:13 that is at least 18 amino acid residues in length, or (vi) that is at least 85% identical to said C-terminal portion.

The mutation can be a single base substitution. The introducing can include introducing nucleic acid encoding the chimeric polypeptide into the cell. The nucleic acid interacting domain can be N-terminal to the facilitating domain. The nucleic acid interacting domain can include a TALE DNA binding domain. The nucleic acid interacting domain can include a zinc finger DNA binding domain. The nucleic acid interacting domain can include CRISPR/Cas DNA binding components. The chimeric polypeptide can further include a linker between the nucleic acid interacting domain and the facilitating domain. The chimeric polypeptide can further include a MTS. The MTS can be an isocitrate dehydrogenase 2 MTS, a human COX8A MTS, or a human SOD2 MTS. The MTS can be at the N-terminus of the chimeric polypeptide. The chimeric polypeptide can include, in order from N-terminus to C-terminus, the MTS, the nucleic acid interacting domain, and the facilitating domain, wherein the chimeric polypeptide can further include a linker between the MTS and the nucleic acid interacting domain. The facilitating domain can have intrinsic cytosine deaminase activity. The facilitating domain can lack cytosine deaminase activity, wherein the chimeric polypeptide can further include an extrinsic deaminase domain. The extrinsic deaminase domain can be an adenosine deaminase domain. The adenosine deaminase domain can include an amino acid sequence at least 90 percent identical to the amino acid sequence set forth in SEQ ID NO: 53. The extrinsic deaminase domain can be a cytosine deaminase domain. The chimeric polypeptide can include, in order from N-terminus to C-terminus, the nucleic acid interacting domain, the facilitating domain, and the extrinsic deaminase domain. The chimeric polypeptide can further include a MTS at the N-terminus of the chimeric polypeptide, a first linker between the nucleic acid interacting domain and the facilitating domain, and a second linker between the facilitating domain and the extrinsic deaminase domain.

In some cases, the method can include introducing a first chimeric polypeptide and a second chimeric polypeptide into the cell, wherein the facilitating domain of the first chimeric polypeptide includes the N-terminal portion of the amino acid sequence set forth in SEQ ID NO:2 that is at least 100 amino acid residues in length, or the amino acid sequence that is at least 92 percent identical to the N-terminal portion; the N-terminal portion of the amino acid sequence set forth in SEQ ID NO:3 that is at least 100 amino acid residues in length, or the amino acid sequence that is at least 85 percent identical to the N-terminal portion; the N-terminal portion of the amino acid sequence set forth in SEQ ID NO:4 that is at least 105 amino acid residues in length, or the amino acid sequence that is at least 85 percent identical to the N-terminal portion; the N-terminal portion of the amino acid sequence set forth in SEQ ID NO:5 that is at least 90 amino acid residues in length, or the amino acid sequence that is at least 85 percent identical to the N-terminal portion; the N-terminal portion of the amino acid sequence set forth in SEQ ID NO:6 that is at least 110 amino acid residues in length, or the amino acid sequence that is at least 85 percent identical to the N-terminal portion; the N-terminal portion of the amino acid sequence set forth in SEQ ID NO:7 that is at least 100 amino acid residues in length, or the amino acid sequence that is at least 85 percent identical to the N-terminal portion; or the N-terminal portion of the amino acid sequence set forth in SEQ ID NO:8 that is at least 100 amino acid residues in length, or the amino acid sequence that is at least 85 percent identical to the N-terminal portion, and wherein the facilitating domain of the second chimeric polypeptide includes the C-terminal portion of the amino acid sequence set forth in SEQ ID NO:2 that is at least 25 amino acid residues in length, or the amino acid sequence that is at least 92 percent identical to the C-terminal portion; the C-terminal portion of the amino acid sequence set forth in SEQ ID NO:3 that is at least 25 amino acid residues in length, or the amino acid sequence that is at least 85 percent identical to the C-terminal portion; the C-terminal portion of the amino acid sequence set forth in SEQ ID NO:4 that is at least 25 amino acid residues in length, or the amino acid sequence that is at least 85 percent identical to the C-terminal portion; the C-terminal portion of the amino acid sequence set forth in SEQ ID NO:5 that is at least 20 amino acid residues in length, or the amino acid sequence that is at least 85 percent identical to the C-terminal portion; the C-terminal portion of the amino acid sequence set forth in SEQ ID NO:6 that is at least 20 amino acid residues in length, or the amino acid sequence that is at least 85 percent identical to the C-terminal portion; the C-terminal portion of the amino acid sequence set forth in SEQ ID NO:7 that is at least 20 amino acid residues in length, or the amino acid sequence that is at least 85 percent identical to the C-terminal portion; or the C-terminal portion of the amino acid sequence set forth in SEQ ID NO:8 that is at least 20 amino acid residues in length, or the amino acid sequence that is at least 85 percent identical to the C-terminal portion.

In some cases, the facilitating domain of the first chimeric polypeptide can include the N-terminal portion of the amino acid sequence set forth in SEQ ID NO:2 that is at least 100 amino acid residues in length, or the amino acid sequence that is at least 92 percent identical to the N-terminal portion, and the facilitating domain of the second chimeric polypeptide can include the C-terminal portion of the amino acid sequence set forth in SEQ ID NO:2 that is at least 25 amino acid residues in length, or the amino acid sequence that is at least 92 percent identical to the C-terminal portion; or the facilitating domain of the first chimeric polypeptide can include the N-terminal portion of the amino acid sequence set forth in SEQ ID NO:3 that is at least 100 amino acid residues in length, or the amino acid sequence that is at least 85 percent identical to the N-terminal portion, and the facilitating domain of the second chimeric polypeptide can include the C-terminal portion of the amino acid sequence set forth in SEQ ID NO:3 that is at least 25 amino acid residues in length, or the amino acid sequence that is at least 85 percent identical to the C-terminal portion; or the facilitating domain of the first chimeric polypeptide can include the N-terminal portion of the amino acid sequence set forth in SEQ ID NO:4 that is at least 105 amino acid residues in length, or the amino acid sequence that is at least 85 percent identical to the N-terminal portion, and the facilitating domain of the second chimeric polypeptide can include the C-terminal portion of the amino acid sequence set forth in SEQ ID NO:4 that is at least 25 amino acid residues in length, or the amino acid sequence that is at least 85 percent identical to the C-terminal portion; or the facilitating domain of the first chimeric polypeptide can include the N-terminal portion of the amino acid sequence set forth in SEQ ID NO:5 that is at least 90 amino acid residues in length, or the amino acid sequence that is at least 85 percent identical to the N-terminal portion, and the facilitating domain of the second chimeric polypeptide can include the C-terminal portion of the amino acid sequence set forth in SEQ ID NO:5 that is at least 20 amino acid residues in length, or the amino acid sequence that is at least 85 percent identical to the C-terminal portion; or the facilitating domain of the first chimeric polypeptide can include the N-terminal portion of the amino acid sequence set forth in SEQ ID NO:6 that is at least 110 amino acid residues in length, or the amino acid sequence that is at least 85 percent identical to the N-terminal portion, and the facilitating domain of the second chimeric polypeptide can include the C-terminal portion of the amino acid sequence set forth in SEQ ID NO:6 that is at least 20 amino acid residues in length, or the amino acid sequence that is at least 85 percent identical to the C-terminal portion; or the facilitating domain of the first chimeric polypeptide can include the N-terminal portion of the amino acid sequence set forth in SEQ ID NO:7 that is at least 100 amino acid residues in length, or the amino acid sequence that is at least 85 percent identical to the N-terminal portion, and the facilitating domain of the second chimeric polypeptide can include the C-terminal portion of the amino acid sequence set forth in SEQ ID NO:7 that is at least 20 amino acid residues in length, or the amino acid sequence that is at least 85 percent identical to the C-terminal portion; or the facilitating domain of the first chimeric polypeptide can include the N-terminal portion of the amino acid sequence set forth in SEQ ID NO:8 that is at least 100 amino acid residues in length, or the amino acid sequence that is at least 85 percent identical to the N-terminal portion, and the facilitating domain of the second chimeric polypeptide can include the C-terminal portion of the amino acid sequence set forth in SEQ ID NO:8 that is at least 20 amino acid residues in length, or the amino acid sequence that is at least 85 percent identical to the C-terminal portion

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an alignment of amino acid sequences of polypeptides from the 13 indicated species; the polypeptide sequences each include a domain having potential facilitating functions and an intrinsic cytidine deaminase domain. From top to bottom: Phosphitispora fastidiosa, SEQ ID NO:13; Burkholderia ubonensis, SEQ ID NO:8; Paraburkholderia guartelaensis, SEQ ID NO:10; Caballeronia, SEQ ID NO:3; Burkholderia cenocepacia, SEQ ID NO:1; Burkholderia gladioli, SEQ ID NO:2; Pseudoduganella violaceinigra, SEQ ID NO:12; Ruminococcus bicirculans, SEQ ID NO: 7; Coriobacteriia bacterium, SEQ ID NO:11; Roseburia intestinalis, SEQ ID NO:4; Treponema, SEQ ID NO:9; Falcatimonas, SEQ ID NO:5; Clostridium, SEQ ID NO:6. The glutamate residue of the presumed intrinsic cytosine deaminase active site of each polypeptide is shown in the box.

FIG. 2A is a schematic representation of a chimeric polypeptide having an exemplary monomeric format mitochondrial base editor design. In this exemplary design, the nucleic acid interacting domain is depicted as a TAL effector DNA-binding domain (“DNA binding domain”), the facilitating domain is depicted as a meltase (“Mlt”) having an intrinsic cytosine deaminase domain (“dCD”) that is deactivated or removed in this format, and the base editing domain is depicted as an extrinsic adenosine deaminase (“AD”). Each of the amino acid sequences set forth in SEQ ID NOS: 1-8 was modified to lack dCD activity and to create the facilitating domain, which was then fused to the other components (e.g., nucleic acid interacting domain and the extrinsic AD). The depicted target dsDNA (SEQ ID NOs: 23 and 24) is that of a human mitochondrial NDI gene sequence present in 293T cells. FIG. 2B shows the amino acid sequences of the facilitating domains that were modified to lack dCD activity. FIG. 2C is a graph plotting mitochondrial A-to-G base editing efficiency of chimeric polypeptides having the indicated sequences, measured using NGS and EditR. The horizontal dotted line shows the background rates of sequence variation from this approach. Each chimeric polypeptide was tested in three separate biological replicates conducted on separate days. Data are represented as the average plus standard deviation. NTC, non-transfected control. The asterisk in FIG. 2A indicates the position at which the highest level of editing activity occurred.

FIG. 3A is a schematic representation of two chimeric polypeptides having an exemplary split format mitochondrial base editor design. In this exemplary design, a first chimeric polypeptide is designed to have a first nucleic acid interacting domain, which is depicted as a TAL effector DNA-binding domain (“DNA binding domain”), and a first facilitating domain, which is depicted as a meltase (“Mlt”) having a first portion of an intrinsic cytosine deaminase domain (“Split CD”) that is not active. In addition, a second chimeric polypeptide is designed to have a second nucleic acid interacting domain, which is depicted as a TAL effector DNA-binding domain (“DNA binding domain”), and a second facilitating domain, which is depicted as a meltase (“Mlt”) having a second portion of an intrinsic cytosine deaminase domain (“Split CD”) that is not active. A functional base editing domain is formed when the first and second portions of the intrinsic cytosine deaminase domain come together based on the DNA binding to the two different nucleic acid interacting domains. Each chimeric polypeptide also can be designed to include an optional inhibitor of uracil DNA glycosylase (“UGI”) to inhibit the base excision repair that changes uracil back to cytosine. The depicted target dsDNA (SEQ ID NOs: 33 and 34) is that of a human mitochondrial ND4 gene sequence present in 293T cells. FIG. 3B shows the sequences for each of the first and second “Split CD” polypeptides that were generated from the sequences set forth in SEQ ID NOS: 1-8. FIG. 3C is a graph plotting mitochondrial C-to-T base editing efficiency of pairs of chimeric polypeptides having the indicated sequences, measured using NGS and EditR. The horizontal dotted line shows the background rates of sequence variation from this approach. Each pair of chimeric polypeptides was tested in three separate biological replicates conducted on separate days. Data are represented as the average plus standard deviation. NTC, non-transfected control. The asterisk in FIG. 3A indicates the position at which the highest level of editing activity occurred.

FIG. 4A is a schematic representation of a split format mitochondrial base editor design. The meltase+CD domain of the construct was replaced with selected sequences to measure intrinsic C-to-T activity. Each selected sequence was divided into two halves to avoid potential cellular toxicity. In this design, the left and right arm contained the C and N-terminal portions, respectively, of the protein. The human mitochondrial ND4 gene was targeted for this base editing experimental test paradigm using 293T cells. FIG. 4B is a graph plotting levels of mitochondrial base editing as measured using NGS and EditR. The dotted line indicates the background rates of sequence variation from this approach. Each meltase was tested in three separate biological replicates conducted on separate days. Data are represented as average plus standard deviation.

FIG. 5A is a schematic representation of a monomeric format mitochondrial A-to-G base editor design. Each test meltase domain included a protein modification to greatly reduce and/or remove any intrinsic cytosine deaminase (dCD). Each candidate meltase construct was fused with an extrinsic Adenosine Deaminase (AD) to provide the net A-to-G base editing activity. The human mitochondrial NDI gene was targeted for this base editing experiment using human primary fibroblast cells, and the base editors were delivered as synthetic RNA. FIG. 5B is a graph plotting levels of mitochondrial base editing as measured using EditR. The dotted line indicates the background rates of sequence variation from this approach. Each meltase was tested in three separate biological replicates conducted on separate days. Data are represented as average plus standard deviation.

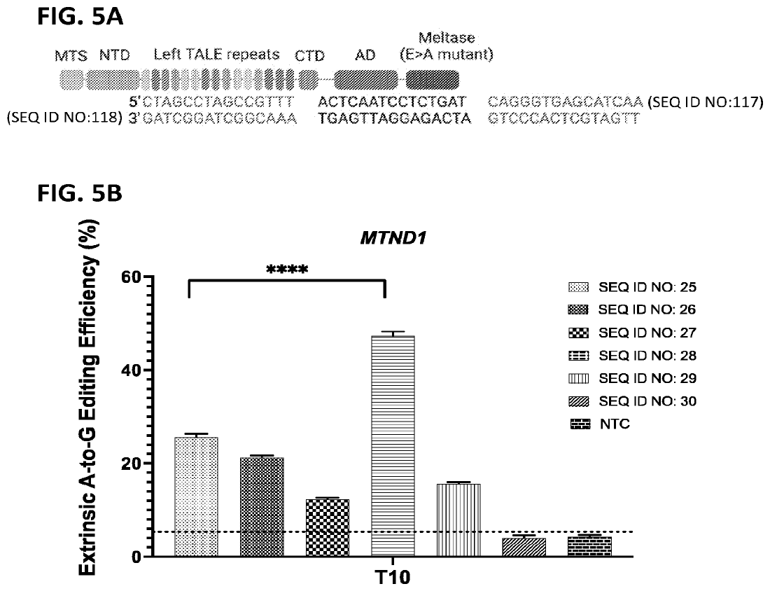

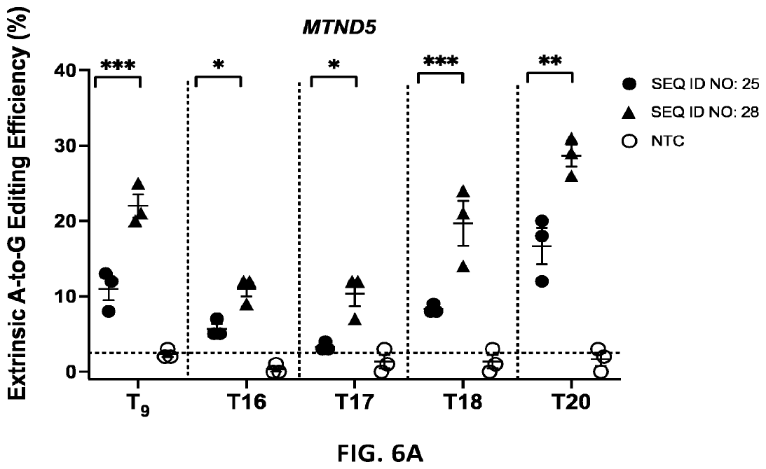

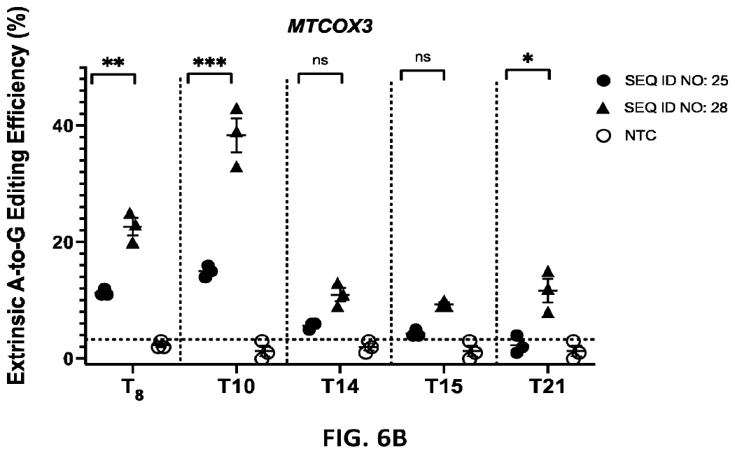

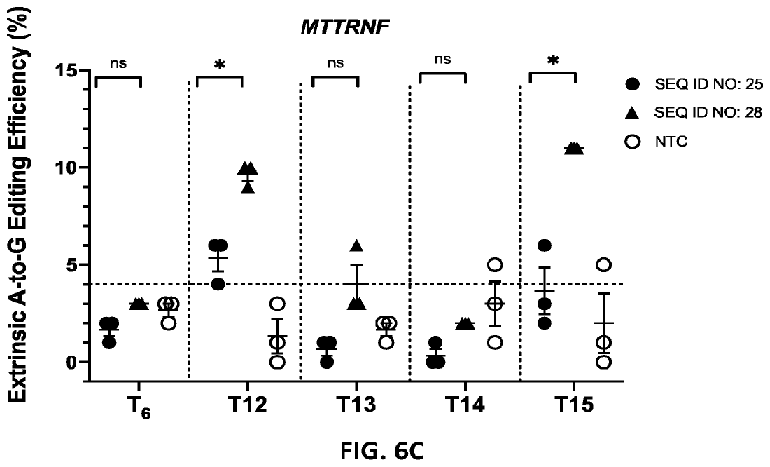

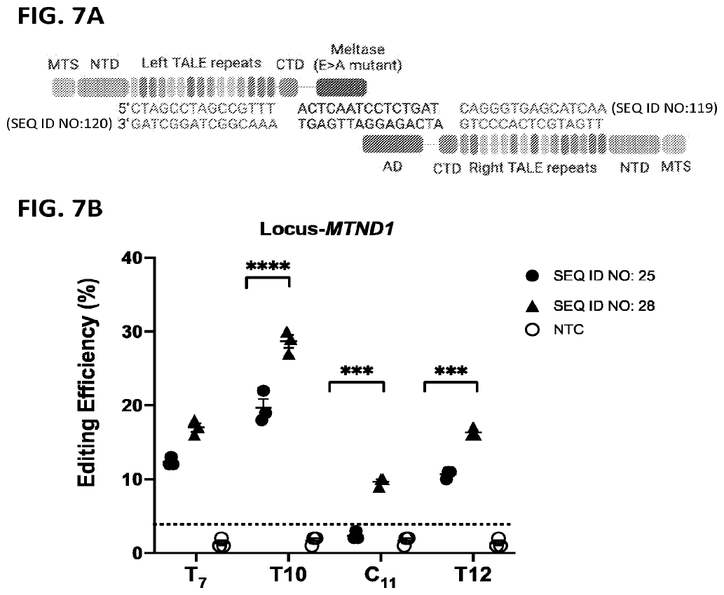

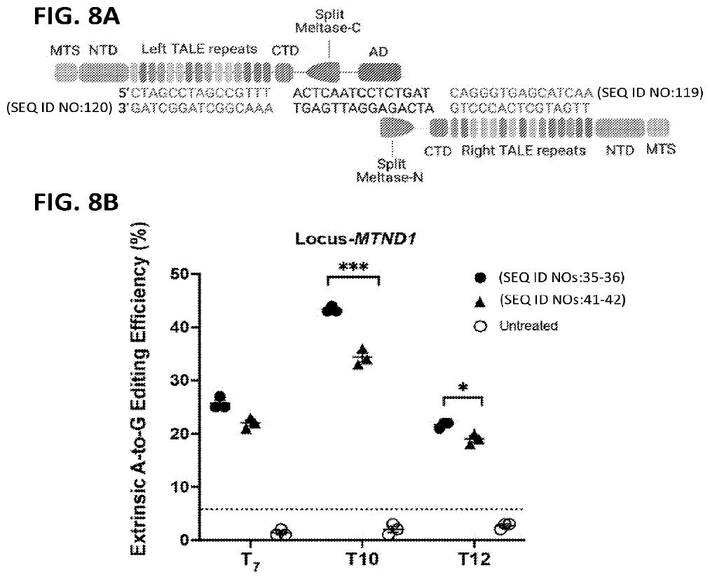

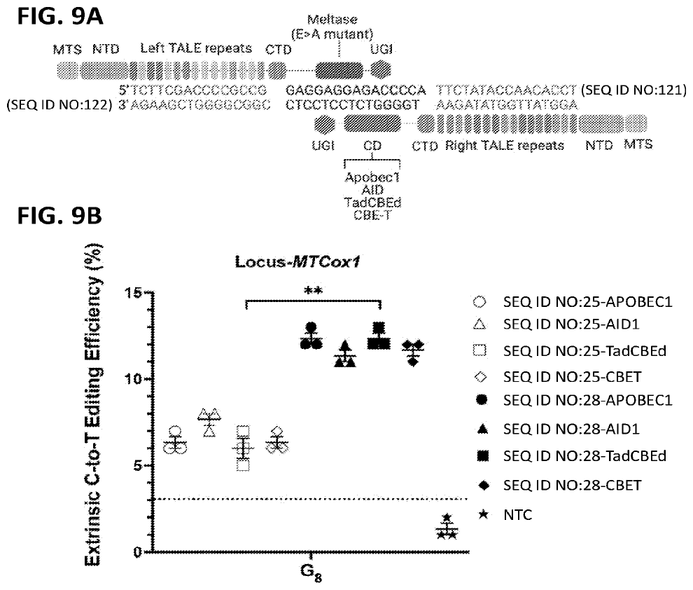

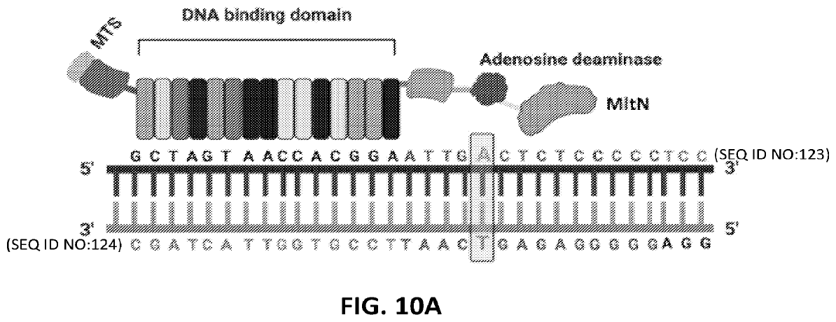

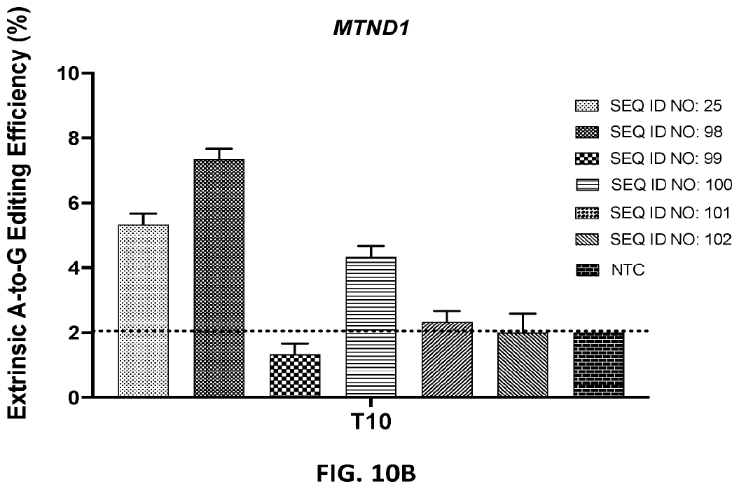

For FIGS. 6A-6D, the monomeric format mitochondrial A-to-G base editor (FIG. 5A) was used on three additional but with delivery as synthetic mRNA. Mitochondrial base editing was measured using Sanger sequencing. The parallel dotted line indicates the estimated background rates of sequence variation from this approach. Each meltase was tested in three separate biological replicates conducted on separate days. Data are represented as the average plus standard deviation. Human mitochondrial ND5 (FIG. 6A), COX3 (FIG. 6B), and TRNF (FIG. 6C) genes were targeted for the base editing experiment using human primary fibroblast cells. Data are represented as average plus standard deviation.

FIG. 7A is a schematic representation of a dimeric format mitochondrial A-to-G base editor design. The Mlt_Bc (SEQ ID NO:25) and Mlt_Ri (SEQ ID NO:28) meltase domains each included an E>A mutation to greatly reduce and/or remove any intrinsic cytosine deaminase (dCD). The right TALE was fused with an extrinsic Adenosine Deaminase (AD) to provide net A-to-G base editing activity. The human mitochondrial NDI gene was targeted for this base editing experiment using 293T cells. FIG. 7B is a graph plotting mitochondrial base editing outcomes as assessed using NGS and EditR. The dashed line represents background rates of sequence variation observed in this approach. This editing experiment demonstrated Mlt_Ri's enhanced meltase activity compared to Mlt_Bc in the dimeric design. The results are presented as the average plus standard deviation.

FIG. 8A is a schematic representation of a split format mitochondrial A-to-G base editor design. The Mlt_Bc (SEQ ID NO:25) and Mlt_Ri (SEQ ID NO:28) meltase domains were split and the Adenosine Deaminase (AD) was tethered to the N-terminal portion of each meltase and programmed to bind the right side of the target. Both right and left TALEs were co-transfected to assess the net A-to-G base editing activity. The experiment targeted the human mitochondrial NDI gene using HEK293T cells. FIG. 8B is a graph plotting mitochondrial base editing outcomes, assessed using NGS and EditR. The dashed line represents background rates of sequence variation observed in this approach. This editing experiment demonstrated that Mlt_Bc perfomed better in this format at the mitochondrial NDI locus. The results are presented as average with standard deviation.

FIG. 9A is a schematic representation of a meltase-assisted extrinsic C-to-T design. The E>A mutated Mlt_Bc (SEQ ID NO:25) and Mlt_Ri (SEQ ID NO:28) meltase domains were tethered to the left TALE arm. The right TALE was fused with four individual Cytosine Deaminases (CDs) to enable net extrinsic C-to-T base editing activity. UGI molecules were added to the constructs to enhance the net C-to-T editing. This experiment targeted the human mitochondrial Coxl gene using HEK293T cells. FIG. 9B is a graph plotting mitochondrial base editing outcomes using NGS and EditR. The dashed line represents background rates of sequence variation observed in this approach. This editing experiment again showcased Mlt_Ri′s enhanced meltase activity over Mlt_Bc in this design, as every Mlt_Ri-assisted CD exhibited higher activity compared to its respective Mlt_Bc counterpart. The results are presented as average with standard deviation.

FIG. 10A is a schematic representation of a chimeric polypeptide having an exemplary monomeric format mitochondrial base editor design. In this exemplary design, the nucleic acid interacting domain is depicted as a TAL effector DNA-binding domain (“DNA binding domain”), the facilitating domain is depicted as a meltase (“MltN”) having an intrinsic cytosine deaminase domain that is deactivated or removed, and the base editing domain is depicted as an extrinsic adenosine deaminase. Each of the amino acid sequences set forth in SEQ ID NOS: 9-13 was modified to reduce or abolish cytosine deaminase activity and to create the facilitating domain, which was then fused to the other components (e.g., a nucleic acid interacting domain and the extrinsic adenosine deaminase). The depicted target dsDNA (SEQ ID NOs: 123 and 124) is that of a human mitochondrial ND1 gene sequence present in 293T cells. FIG. 10B is a graph plotting mitochondrial A-to-G base editing efficiency of chimeric polypeptides having the indicated MltN sequences, measured using NGS and EditR. Each chimeric polypeptide was tested in three separate biological replicates conducted on separate days. Data are represented as the average plus standard deviation. NTC, non-transfected control.

### DETAILED DESCRIPTION

DddAₜₒₓ is a bacterial toxin with the unique biochemical property of functioning as a dual component molecule that binds double-stranded DNA for subsequent use as a substrate by an intrinsically encoded cytosine deaminase. The use of DddAₜₒₓ as a DNA base editor to generate C-to-T mutations in mitochondria (in conjunction with programmable transcription activator-like enhancer (TALE) DNA binding proteins) is described elsewhere (Mok et al., Nature, 583 (7817): 631-637 (2020); and Mok et al., Nature Biotechnol, 1-10, (2022)). The DddAₜₒₓ toxin was derived from Burkholderia cenocepacia, and was shown to function even after being split into two halves when localized near each other at a single DNA locus using two different TALE domains. Other studies also have used the DddAₜₒₓ protein molecule to introduce C-to-T mutations in mitochondria (Sabharwal et al., The CRISPR Journal, 4 (6): 799-821 (2021); Lee et al., Nature Commun., 12 (1): 1-6 (2021); Kar et al., STAR Protocols, 3 (2): 101288 (2022); and Guo et al., Cell Discov., 7 (1): 1-5 (2021)). DddAₜₒₓ's function of enabling the use of dsDNA as a substrate is separable from the intrinsic deaminase. In particular, targeted amino acid substitutions were introduced into the cytosine deaminase active site to yield a “dead” toxin that was fused with a separate deaminase, resulting in the generation of a mitochondrial base editor that can introduce A-to-G substitutions in mtDNA (Cho et al., Cell, 185 (10): 1764-1776 (2022)).

This document provides chimeric polypeptides that, in general, can include a nucleic acid interacting domain that is targeted to a specific nucleotide sequence, a facilitating domain, and a base editing domain (e.g., a deaminase domain). Such chimeric polypeptides can have the ability to bind to dsDNA and facilitate and carry out subsequent and separable programmable DNA modifications such as single base editing.

Any appropriate nucleic acid interacting domain can be included in the chimeric polypeptides provided herein, to target the chimeric polypeptides to specific nucleotide sequences. In some cases, a nucleic acid interacting domain can interact with a specific DNA sequence. Examples of nucleic acid interacting domains that can be included in the chimeric polypeptides provided herein include, without limitation, zinc finger domains (e.g., a zinc finger DNA binding domain derived from the mouse transcription factor Zif268; see, e.g., Porteus and Baltimore, Science, 300:763 (2003)), TALE domains, and CRISPR/Cas domains.

In some cases, chimeric polypeptides can be designed to include a nucleic acid interacting domain derived from a TALE. TALEs are polypeptides of plant pathogenic bacteria that are injected by the pathogen into the plant cell, where they travel to the nucleus and function as transcription factors to turn on specific plant genes (see, e.g., Gu et al., Nature, 435:1122 (2005); Yang et al., Proc. Natl. Acad. Sci. USA, 103:10503 (2006); Kay et al., Science, 318:648 (2007); Sugio et al., Proc. Natl. Acad. Sci. USA, 104:10720 (2007); and Römer et al., Science, 318:645 (2007)). The specificity of DNA binding is conferred by an effector-variable number of consecutive, imperfect, typically 34 amino acid repeats (Schornack et al., J. Plant Physiol., 163:256 (2006)). Polymorphisms that are present primarily at repeat positions 12 and 13 (referred to as the repeat variable-diresidue or RVD) correspond to the individual nucleotides in the TALE target sites in a direct, linear fashion (one RVD to one nucleotide) with some degeneracy and no apparent context dependency. Since the primary amino acid sequence of a TALE dictates the nucleotide sequence to which it binds, TALEs can be designed for the purpose of binding to particular nucleotide sequences. For example, by linking a TALE to a base editing domain, a sequence-specific TALE base editor can be designed and generated to recognize a preselected target nucleotide sequence present in a cell (e.g., in the mitochondria or the nucleus of a cell).

When using a TALE-derived nucleic acid interacting domain as the nucleic acid interacting domain of a chimeric polypeptide provided herein, the TALE-derived nucleic acid interacting domain within the chimeric polypeptide can include any appropriate number of repeat sequences. For example, a TALE-derived nucleic acid interacting domain of a chimeric polypeptide provided herein can include about 15 to about 20 (e.g., 15, 16, 17, 18, 19, or 20) repeat sequences, such that the target nucleotide sequence for the chimeric polypeptide is about 15 to about 20 nucleotides in length. In some cases, a chimeric polypeptides provided herein can be designed to include additional TALE sequences that flank the repeat region that contains the nucleic acid interacting domain. For example, a chimeric polypeptide provided herein can include a TALE DNA binding domain flanked by an N-terminal TALE sequence and/or a C-terminal TALE sequence. The N- and C-terminal TALE DNA binding domain flanking sequences can have any appropriate length. For example, a N-terminal TALE DNA binding domain flanking sequence can have a length between about 80 to about 200 amino acids (e.g., about 80 to about 100 amino acids, about 100 to about 120 amino acids, about 120 to about 140 amino acids, about 130 to about 150 amino acids, about 140 to about 160 amino acids, about 150 to about 170 amino acids, about 160 to about 180 amino acids, or about 180 to about 200 amino acids). A C-terminal TALE DNA binding domain flanking sequence can have a length between about 10 to about 80 amino acids (e.g., about 10 to about 20 amino acids, about 20 to about 30 amino acids, about 30 to about 40 amino acids, about 40 to about 50 amino acids, about 50 to about 60 amino acids, about 60 to about 70 amino acids, or about 70 to about 80 amino acids). Moreover, the TALE portion of a chimeric polypeptide provided herein can include one or more additional variations (e.g., substitutions, deletions, or additions) as compared to a wild type TALE sequence. A representative TALE DNA binding domain is set forth in SEQ ID NO:51. The N-terminal and C-terminal flanking sequences on either side of the repeat domain are underlined, and the RVD within each repeat is in italics.

(SEQ ID NO: 51)
GIRIQDLRTLGYSQQQQEKIKPKVRSTVAQHHEALVGHGFTHAHIVALS
QHPAALGTVAVKYQDMIAALPEATHEAIVGVGKOWSGARALEALLTVAG
ELRGPPLQLDTGOLLKIAKRGGVTAVEAVHAWRNALTGAPLNLTPDQVV
AIASHDGGKQALETVQRLLPVLCQAHGLTPDQVVAIASNGGGKQALETV
QRLLPVLCQDHGLTPAQVVAIASNIGGKQALETVQRLLPVLCQDHGLTP
DQVVAIASNNGGKQALETVQRLLPVLCQAHGLTPDQVVAIASHDGGKQA
LETVQRLLPVLCQDHGLTPDQVVAIASHDGGKQALETVQRLLPVLCQAH
GLTPAQVVAIASNGGGKQALETVQRLLPVLCQDHGLTPAQVVAIASNIG
GKQALETVQRLLPVLCQAHGLTPDQVVAIASNNGGKQALETVQRLLPVL
CQAHGLTPAQVVAIASHDGGKQALETVQRLLPVLCQDHGLTPDQVVAIA
SHDGGKQALETVQRLLPVLCQAHGLTPEQVVAIASNNGGKQALETVQRL
LPVLCQAHGLTPAQVVAIASNGGGKQALETVQRLLPVLCQAHGLTPAQV
VAIASNGGGKQALETVQRLLPVLCQAHGLTPEQVVAIASNGGGKQALES
IVAQLSRPDPALAALLV

In some cases, a chimeric polypeptide provided herein can be designed to include a nucleic acid interacting domain from a Clustered Regularly Interspersed Short Palindromic Repeats (CRISPR)/CRISPR associated system (Cas) system. In its native context, the CRISPR/Cas system provides bacteria and archaea with immunity to invading foreign nucleic acids (Jinek et al., Science, 337:816-821 (2012)). The CRISPR/Cas system relies on (a) small RNAs that base-pair with sequences carried by invading nucleic acid, and (b) a specialized class of Cas endonucleases that cleave nucleic acids complementary to the small RNA. Thus, the CRISPR/Cas system uses base pairing directed by the CRISPR RNAs to direct DNA or RNA cleavage by the Cas nuclease. The CRISPR/Cas system can be reprogrammed to create targeted double-strand DNA breaks in higher eukaryotic genomes, including animal and plant cells (Mali et al., Science, 339:823-826 (2013); and Li et al., Nature Biotechnol., 31 (8): 688-691 (2013)). Further, by modifying specific amino acids in the Cas protein that are responsible for DNA cleavage, the nuclease activity can be attenuated or ablated. For example, a Cas9 polypeptide can include a D10A mutation and a H840A mutation, such that the Cas9 nuclease is inactivated. The amino acid sequence of a representative inactivated Cas9 polypeptide from Streptococcus pyogenes is set forth in SEQ ID NO:52, with the amino acid residues at positions 10 and 840 underlined and in bold:

(SEQ ID NO: 52)
MDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

In addition to nucleic acid interacting domains, chimeric polypeptides provided herein also can include a facilitating domain that can enhance the activity of base editing by the chimeric polypeptides. The facilitating domain of a chimeric polypeptide provided herein can be any appropriate facilitating domain. In general, the facilitating domain of a chimeric polypeptide provided herein is intrinsically coupled to a cytidine deaminase domain or a portion thereof. In some cases, the cytidine deaminase domain can have cytidine deaminase activity. In some cases, the cytidine deaminase domain can include one or more mutations that reduce or abolish the cytidine deaminase activity, in which case the chimeric polypeptide also can include an extrinsic base editing domain (e.g., an adenosine deaminase domain) as described herein.

Examples of amino acid sequences of facilitating domains having potential facilitating functions and including an intrinsic cytidine deaminase domain as a base editing domain are set forth in SEQ ID NOS: 1-13:

Burkholderia cenocepacia
(SEQ ID NO: 1)
GSYALGPYQISAPQLPAYNGQTVGTFYYVNDAGGLESKVFSSGGPTPYP
NYANAGHVEGQSALFMRDNGISEGLVFHNNPEGTCGFCVNMTETLLPEN
AKMTVVPPEGAIPVKRGATGETKVFTGNSNSPKSPTKGGC
Burkholderia gladioli
(SEQ ID NO: 2)
GSYTLGSYQISAPQLPAYNGQTVGTFYYVNDAGGLESRTFSSGGPTPYP
NYANAGHVEGQSALFMRDNGISDGLVFHNNPEGTCGFCVNMTETLLPEN
SKLTVVPPEGSIPVKRGATGETRTFTGNSKSPKSPVKGGC
Caballeronia sp.
(SEQ ID NO: 3)
TETFPLGSNQIPRPGLPAYDGKTSGVFYYLDGDGGMQSKVFTSGGVTEF
PNYANAGHVEGQSALFMRSNGVGEGVVFHNNPEGTCGFCVSMTETLLPE
NAKLTVVPPEGAVPVKRGATGDVRVFTGNKNSPKSPSSGGAC
Roseburia intestinalis
(SEQ ID NO: 4)
SQYPCKEEMSAGAGESGRKTISLPEYDGTTTHGVLVLDDGTQIGFTSGN
GDPRYTNYRNNGHVEQKSALYMRENNISNATVYHNNINGTCGYCNTMTA
TFLPEGATLTVVPPENAVANNSRAIDYVKTYTGTSNDPKISPRYKGN
Falcatimonas sp.
(SEQ ID NO: 5)
GGSSSINLPEYDGKTTHGVLVLDDGTQVPFSSGNANPNYKNYIPASHVE
GKSAIYMRENGINNGTVFHNNTDGTCPYCDKMLPTLLEEGSTLTVVPPA
NANAPKPSWVDTVKTYIGNDKIPKKPK
Clostridium sp.
(SEQ ID NO: 6)
QSNMSITDRLAKQKEKQDNTNIIDNRPKLPDYDGKTTHGILVTPNSEHI
PFSSGNPNPNYKNYIPASHVEGKSAIYMRENGITSGTIYYNNTDGTCPY
CDKMLSTLLEEGSVLEVIPPINAKAPKPSWVDKPKTYIGNNKVPKPNK
Ruminococcus bicirculans
(SEQ ID NO: 7)
NANCNQEKPVLPKYDGKTTEGVMVTPDGKQISFKSGNSSTPSYPQYKAQ
SASHVEGKAALYMRENGINEATVFHNNPNGTCGFCDRQVPALLPKGAKL
TVVPPSNSVANNVRAIPVPKTYIGNSTVPKIK
Burkholderia ubonensis
(SEQ ID NO: 8)
ALLREAYPSMEGATLPPFDGKTTIGLMFYTDASGQYQVKKLFSGEKVLS
NYDATGHVEGKAALIMRNEKITEAVVMHNHPSGTCNYCDKQVETLLPKN
ATLRVIPPENAKAPTSYWNDQPTTYRGDGKDPKAPSKK
Treponema sp.
(SEQ ID NO: 9)
SNNEKKKNANKQKIELPPYDGKTTYGVLILDDGKQYSFNSGKPAPIYRN
YIPASHVEGKAAIYMRENKIQSGTVYHNNTDGTCPYCDKMLPTLLEKDS
TLKVVPPQNATSSKKGWITNEKIYIGNDKIPKTAR
Paraburkholderia guartelaensis
(SEQ ID NO: 10)
REDALLREQFPSMDAVTLPPFDGKTTIGYMFYTDANGQYHVRKLYSGGK
VLSNYDSSGHVEGMAALIMRKGRITEAVVMHNHPSGTCHYCNGQVETLL
PKNAKLKVIPPANAKAPTKYWYDQPVDYLGNSNDPKPPS
Coriobacteriia bacterium
(SEQ ID NO: 11)
SGFYQPANIPSSEVVLPEFDGKTTYGELRTPDGKSIPLQSGDPDPQYSN
YVSSSHVEGKAAQYMRENGIEQATVYHNNANGTCGYCDKMLPTLLPDGS
ELTVIPPASAVPNNPQAVAAPKTYTGNSAVPKTNPRFK
Pseudoduganella violaceinigra
(SEQ ID NO: 12)
GTNAAGSSGKNVRMPRDYASELPEYDGKTTHGVLVTNEGKVIQLRSGGK
EEPYTGYKAVSASHVEGKAAIWIRENGSSGGTVYHNNTTGTCGYCNSQV
KALLPEGVELKIVPPTNAVAKNAQARAVPTINVGNGTQPGRKQK
Phosphitispora fastidiosa
(SEQ ID NO: 13)
SRPRTPEDESIAAAIAERGKMTPAPKGKTSASVEGNSTESGWTTGPRAA
ENTEAVMELSKKMGHDLQPNKLLDQGKPGRYHASHAEKQAAVAAPNKPI
AVSAPMCDNCRRFFRALARYTGKPQTVAEPRAVWVFRPDGSVLVVPK

In some cases, a facilitating domain can be engineered to contain amino acid sequences (or portions thereof) from two or more other facilitating domains. Examples of such engineered facilitating domains that can have potential facilitating functions and can include a cytidine deaminase domain as a base editing domain are set forth in SEQ ID NOS: 14-22:

Engineered protein sequence 1
(SEQ ID NO: 14)
TETFPLGSNQIPRPGLPAYDGKTSGVFYYLDGDGGMQSKVFTSGNGDPR
YTNYRNNGHVAQKSALYMRENNISNATVYHNNTNGTCGYCNTMTATFLP
EGATLTVVPPENAVANNSRAIDYVKTYTGTSNDPKISPRYKGN
Engineered protein sequence 2
(SEQ ID NO: 15)
TETFPLGSNQIPRPGLPAYDGKTSGVFYYLDGDGGMQSKVFKSGNSSTP
SYPQYKAQSASHVAGKAALYMRENGINEATVFHNNPNGTCGFCDRQVPA
LLPKGAKLTVVPPSNSVANNVRAIPVPKTYIGNSTVPKIK
Engineered protein sequence 3
(SEQ ID NO: 16)
SQYPCKEEMSAGAGESGRKTISLPEYDGTTTHGVLVLDDGTQIGFKSGN
SSTPSYPQYKAQSASHVAGKAALYMRENGINEATVFHNNPNGTCGFCDR
QVPALLPKGAKLTVVPPSNSVANNVRAIPVPKTYIGNSTVPKIK
Engineered protein sequence 4
(SEQ ID NO: 17)
TETFPLGSNQIPRPGLPAYDGKTSGVFYYLDGDGGMQSKVFTSGGVTEF
PNYANAGHVAGQSALFMRSNGVGEGVVYHNNTNGTCGYCNTMTATFLPE
GATLTVVPPENAVANNSRAIDYVKTYTGTSNDPKISPRYKGN
Engineered protein sequence 5
(SEQ ID NO: 18)
TETFPLGSNQIPRPGLPAYDGKTSGVFYYLDGDGGMQSKVFTSGGVTEF
PNYANAGHVAGQSALFMRSNGVGEGVVFHNNPNGTCGFCDRQVPALLPK
GAKLTVVPPSNSVANNVRAIPVPKTYIGNSTVPKIK
Engineered protein sequence 6
(SEQ ID NO: 19)
SQYPCKEEMSAGAGESGRKTISLPEYDGTTTHGVLVLDDGTQIGFTSGN
GDPRYTNYRNNGHVAQKSALYMRENNISNATVFHNNPNGTCGFCDRQVP
ALLPKGAKLTVVPPSNSVANNVRAIPVPKTYIGNSTVPKIK
Engineered protein sequence 7
(SEQ ID NO: 20)
TETFPLGSNQIPRPGLPAYDGKTSGVFYYLDGDGGMQSKVFTSGGVTEF
PNYANAGHVAGQSALFMRSNGVGEGVVFHNNPEGTCGFCVSMTETLLPE
NAKLTVVPPENAVANNSRAIDYVKTYTGTSNDPKISPRYKGN
Engineered protein sequence 8
(SEQ ID NO: 21)
TETFPLGSNQIPRPGLPAYDGKTSGVFYYLDGDGGMQSKVFTSGGVTEF
PNYANAGHVAGQSALFMRSNGVGEGVVFHNNPEGTCGFCVSMTETLLPE
NAKLTVVPPSNSVANNVRAIPVPKTYIGNSTVPKIK
Engineered protein sequence 9
(SEQ ID NO: 22)
SQYPCKEEMSAGAGESGRKTISLPEYDGTTTHGVLVLDDGTQIGFTSGN
GDPRYTNYRNNGHVAQKSALYMRENNISNATVYHNNTNGTCGYCNTMTA
TFLPEGATLTVVPPSNSVANNVRAIPVPKTYIGNSTVPKIK

As described herein, a chimeric polypeptide provided herein can be designed to include a facilitating domain having an amino acid sequence that includes the amino acid sequence set forth in SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO:11, SEQ ID NO:12, or SEQ ID NO:13. In some cases, a chimeric polypeptide provided herein can be designed to include a facilitating domain having an amino acid sequence that is based on the amino acid sequence set forth in SEQ ID NO:2, SEQ ID NO:3, SEQ ID NO: 4, SEQ ID NO:5, SEQ ID NO:6, SEQ ID NO: 7, or SEQ ID NO:8, but is less than 100 percent identical to the amino acid sequence set forth in SEQ ID NO:2, SEQ ID NO: 3, SEQ ID NO:4, SEQ ID NO:5, SEQ ID NO:6, SEQ ID NO:7, SEQ ID NO:8, SEQ ID NO: 9, SEQ ID NO: 10, SEQ ID NO:11, SEQ ID NO: 12, or SEQ ID NO: 13.

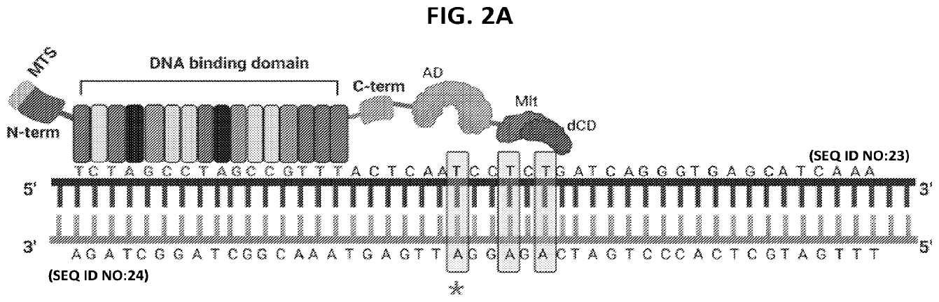

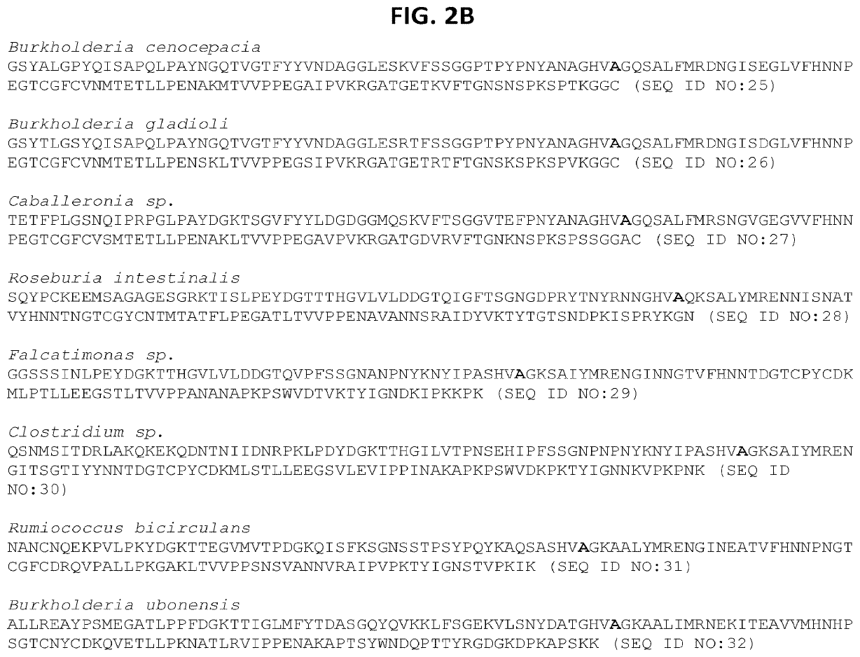

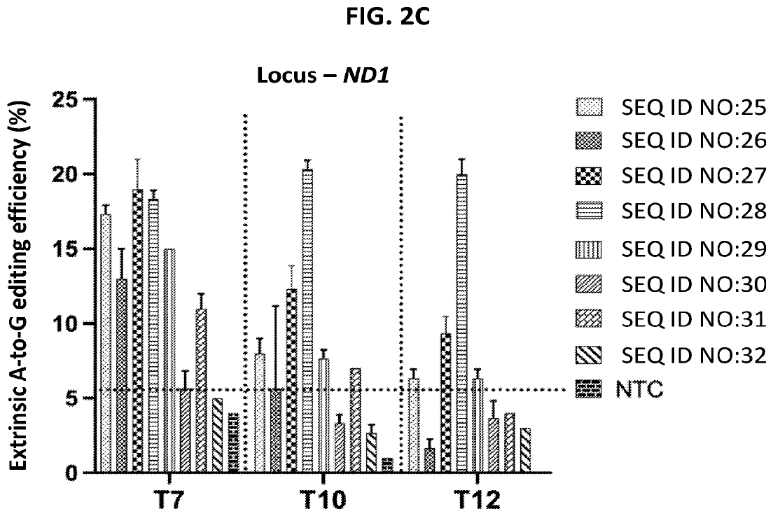

For example, a chimeric polypeptide provided herein can be designed to include a facilitating domain having an amino acid sequence that is at least 92 percent identical (e.g., at least 93 percent, at least 94 percent, at least 95 percent, at least 96 percent, at least 97 percent, at least 98 percent, or at least 99 percent identical, or 100 percent identical) to the amino acid sequence set forth in SEQ ID NO:2. In some cases, a chimeric polypeptide provided herein can be designed to include the amino acid sequence set forth in SEQ ID NO:2 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having the amino acid sequence set forth in SEQ ID NO:26 (FIG. 2B), in which an alanine amino acid residue was substituted for the glutamate amino acid residue at position 58 of SEQ ID NO: 2. Other examples of amino acid additions, subtractions, or substitutions that can be made within SEQ ID NO:2 include, without limitation, substitution of a serine residue for the glycine residue at position 59 of SEQ ID NO:2.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:3. In some cases, a chimeric polypeptide provided herein can be designed to include SEQ ID NO:3 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having the amino acid sequence set forth in SEQ ID NO:27 (FIG. 2B), in which an alanine amino acid residue was substituted for the glutamate amino acid residue at position 59 of SEQ ID NO:3. Other examples of amino acid additions, subtractions, or substitutions that can be made within SEQ ID NO:3 include, without limitation, substitution of a serine residue for the glycine residue at position 60 of SEQ ID NO:3.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:4. In some cases, a chimeric polypeptide provided herein can be designed to include SEQ ID NO:4 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having the amino acid sequence set forth in SEQ ID NO:28 (FIG. 2B), in which an alanine amino acid residue was substituted for the glutamate amino acid residue at position 64 of SEQ ID NO:4. Other examples of amino acid additions, subtractions, or substitutions that can be made within SEQ ID NO:4 include, without limitation, substitution of a serine residue for the glutamine residue at position 65 of SEQ ID NO:4.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:5. In some cases, a chimeric polypeptide provided herein can be designed to include SEQ ID NO:5 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having the amino acid sequence set forth in SEQ ID NO:29 (FIG. 2B), in which an alanine amino acid residue was substituted for the glutamate amino acid residue at position 49 of SEQ ID NO:5. Other examples of amino acid additions, subtractions, or substitutions that can be made within SEQ ID NO:5 include, without limitation, substitution of a serine residue for the glycine residue at position 50 of SEQ ID NO:5.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:6. In some cases, a chimeric polypeptide provided herein can be designed to include SEQ ID NO:6 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having the amino acid sequence set forth in SEQ ID NO:30 (FIG. 2B), in which an alanine amino acid residue was substituted for the glutamate amino acid residue at position 70 of SEQ ID NO:6. Other examples of amino acid additions, subtractions, or substitutions that can be made within SEQ ID NO:6 include, without limitation, substitution of a serine residue for the glycine residue at position 71 of SEQ ID NO:6.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:7. In some cases, a chimeric polypeptide provided herein can be designed to include SEQ ID NO:7 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having the amino acid sequence set forth in SEQ ID NO:31 (FIG. 2B), in which an alanine amino acid residue was substituted for the glutamate amino acid residue at position 55 of SEQ ID NO:7. Other examples of amino acid additions, subtractions, or substitutions that can be made within SEQ ID NO:7 include, without limitation, substitution of a serine residue for the glycine residue at position 56 of SEQ ID NO:7.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:8. In some cases, a chimeric polypeptide provided herein can be designed to include SEQ ID NO:8 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having the amino acid sequence set forth in SEQ ID NO:31 (FIG. 2B), in which an alanine amino acid residue was substituted for the glutamate amino acid residue at position 58 of SEQ ID NO:8. Other examples of amino acid additions, subtractions, or substitutions that can be made within SEQ ID NO:8 include, without limitation, substitution of a serine residue for the glycine residue at position 59 of SEQ ID NO:8.

In some cases, a chimeric polypeptide provided herein can be designed to include a facilitating domain that includes the amino acid sequence set forth in any of SEQ ID NOs: 9 to 22. In some cases, a chimeric polypeptide provided herein can be designed to include a facilitating domain having an amino acid sequence that is based on the amino acid sequence set forth in any of SEQ ID NOs: 9 to 22, but is less than 100 percent identical to the amino acid sequence set forth in SEQ ID NOs: 9 to 22, respectively.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:9. In some cases, a chimeric polypeptide provided herein can be designed to include SEQ ID NO:9 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having an amino acid sequence in which an alanine amino acid residue was substituted for the glutamate amino acid residue at position 57 of SEQ ID NO:9. Other examples of amino acid additions, subtractions, or substitutions that can be made within SEQ ID NO:9 include, without limitation, substitution of a serine residue for the glycine residue at position 58 of SEQ ID NO:9.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:10. In some cases, a chimeric polypeptide provided herein can be designed to include SEQ ID NO:10 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having an amino acid sequence in which an alanine amino acid residue was substituted for the glutamate amino acid residue at position 61 of SEQ ID NO:10. Other examples of amino acid additions, subtractions, or substitutions that can be made within SEQ ID NO:10 include, without limitation, substitution of a serine residue for the glycine residue at position 62 of SEQ ID NO:10.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:11. In some cases, a chimeric polypeptide provided herein can be designed to include SEQ ID NO:11 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having an amino acid sequence in which an alanine amino acid residue was substituted for the glutamate amino acid residue at position 57 of SEQ ID NO:11. Other examples of amino acid additions, subtractions, or substitutions that can be made within SEQ ID NO:11 include, without limitation, substitution of a serine residue for the glycine residue at position 58 of SEQ ID NO:11.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:12. In some cases, a chimeric polypeptide provided herein can be designed to include SEQ ID NO:12 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having an amino acid sequence in which an alanine amino acid residue was substituted for the glutamate amino acid residue at position 65 of SEQ ID NO:12. Other examples of amino acid additions, subtractions, or substitutions that can be made within SEQ ID NO: 12 include, without limitation, substitution of a serine residue for the glycine residue at position 66 of SEQ ID NO:12.

In some cases, a chimeric polypeptide provided herein can be designed to include a facilitating domain having an amino acid sequence that is at least 75 percent identical (e.g., at least 80 percent, at least 85 percent, at least 90 percent, at least 95 percent, at least 96 percent, at least 97 percent, at least 98 percent, or at least 99 percent identical, or 100 percent identical) to the amino acid sequence set forth in SEQ ID NO:13. In some cases, a chimeric polypeptide provided herein can be designed to include SEQ ID NO: 13 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, a chimeric polypeptide provided herein can be designed to include a facilitating domain having an amino acid sequence in which an alanine amino acid residue was substituted for the glutamate amino acid residue at position 86 of SEQ ID NO:13. Other examples of amino acid additions, subtractions, or substitutions that can be made within SEQ ID NO:13 include, without limitation, substitution of a serine residue for the lysine residue at position 87 of SEQ ID NO: 13.

The percent sequence identity between a particular amino acid or nucleic acid sequence and an amino acid or nucleic acid sequence referenced by a particular sequence identification number is determined as follows. First, an amino acid or nucleic acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows:-i is set to a file containing the first nucleic acid sequence to be compared (e.g., C: \seq1.txt);-j is set to a file containing the second nucleic acid sequence to be compared (e.g., C: \seq2.txt);-p is set to blastn;-o is set to any desired file name (e.g., C: \output.txt);-q is set to-1;-r is set to 2; and all other options are left at their default setting. For example, the following command can be used to generate an output file containing a comparison between two sequences: C: \B12seq-i c: \seq1.txt-j c: \seq2.txt-p blastn-o c: \output.txt-q-1-r 2. To compare two amino acid sequences, the options of B12seq are set as follows:-i is set to a file containing the first amino acid sequence to be compared (e.g., C: \seq1.txt);-j is set to a file containing the second amino acid sequence to be compared (e.g., C: \seq2.txt);-p is set to blastp;-o is set to any desired file name (e.g., C: \output.txt); and all other options are left at their default setting. For example, the following command can be used to generate an output file containing a comparison between two amino acid sequences: C: \B12seq-i c: \seq1.txt-j c: \seq2.txt-p blastp-o c: \output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. A matched position refers to a position in which an identical nucleotide or amino acid residue occurs at the same position in aligned sequences. The percent sequence identity is determined by dividing the number of matches by the length of the sequence set forth in the identified sequence (e.g., SEQ ID NO:2), followed by multiplying the resulting value by 100. For example, an amino acid sequence that has 130 matches when aligned with the sequence set forth in SEQ ID NO:2 is 94.2 percent identical to the sequence set forth in SEQ ID NO:2 (i.e., 130+138×100=94.2). It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It also is noted that the length value will always be an integer.

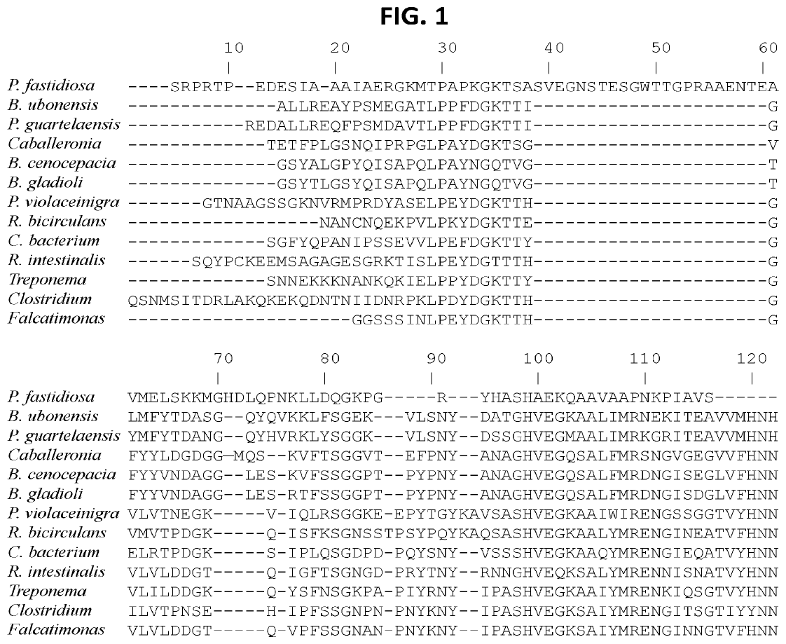

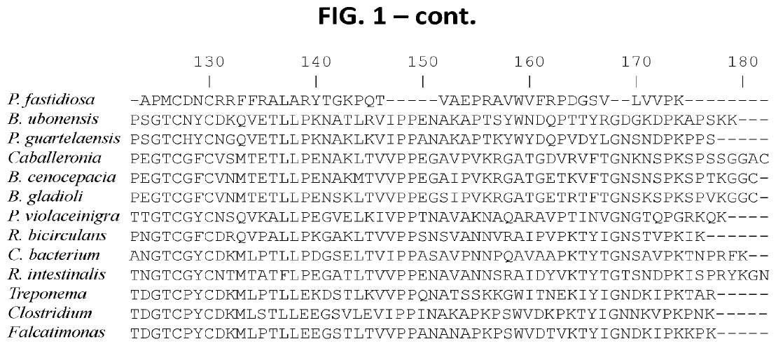

In some cases, a chimeric polypeptide provided herein can be designed to include a facilitating domain that also includes an intrinsic cytidine deaminase domain. In some cases, a chimeric polypeptide provided herein can be designed to include a facilitating domain having one or more mutations that reduce (e.g., eliminate) activity of an intrinsic cytidine deaminase domain. For example, an amino acid residue at (or predicted to be at) the active site of a cytosine deaminase active site can be substituted with another amino acid residue. In some cases, a glutamic acid amino acid residue at (or predicted to be at) the active site of a cytosine deaminase active site can be substituted with another amino acid residue (e.g., an alanine amino acid residue). Examples of such glutamic acid amino acid residues within SEQ ID NOs: 2-8 are shown within the box in FIG. 1. Amino acid sequences corresponding to SEQ ID NOs: 2-8 but having putative active site glutamine residues substituted with alanine are set forth in SEQ ID NOs: 26-32 (FIG. 2B). Amino acid sequences corresponding to SEQ ID NOs: 9-13 but having putative active site glutamine residues substituted with alanine are set forth in SEQ ID NOs: 98-102:

(SEQ ID NO: 98)
SNNEKKKNANKQKIELPPYDGKTTYGVLILDDGKQYSFNSGKPAPIYRN
YIPASHVAGKAAIYMRENKIQSGTVYHNNTDGTCPYCDKMLPTLLEKDS
TLKVVPPQNATSSKKGWITNEKIYIGNDKIPKTAR (based on
Treponema sp. sequence)
(SEQ ID NO: 99)
REDALLREQFPSMDAVTLPPFDGKTTIGYMFYTDANGQYHVRKLYSGGK
VLSNYDSSGHVAGMAALIMRKGRITEAVVMHNHPSGTCHYCNGQVETLL
PKNAKLKVIPPANAKAPTKYWYDQPVDYLGNSNDPKPPS (based on
Paraburkholderia guartelaensis sequence)
(SEQ ID NO: 100)
SGFYQPANIPSSEVVLPEFDGKTTYGELRTPDGKSIPLQSGDPDPQYSN
YVSSSHVAGKAAQYMRENGIEQATVYHNNANGTCGYCDKMLPTLLPDGS
ELTVIPPASAVPNNPQAVAAPKTYTGNSAVPKTNPRFK (based on
Coriobacteriia bacterium sequence)
(SEQ ID NO: 101)
GTNAAGSSGKNVRMPRDYASELPEYDGKTTHGVLVTNEGKVIQLRSGGK
EEPYTGYKAVSASHVAGKAAIWIRENGSSGGTVYHNNTTGTCGYCNSQV
KALLPEGVELKIVPPTNAVAKNAQARAVPTINVGNGTQPGRKQK
(based on Pseudoduganella violaceinigra sequence)
(SEQ ID NO: 102)
SRPRTPEDESIAAAIAERGKMTPAPKGKTSASVEGNSTESGWTTGPRAA
ENTEAVMELSKKMGHDLQPNKLLDQGKPGRYHASHAAKQAAVAAPNKPI
AVSAPMCDNCRRFFRALARYTGKPQTVAEPRAVWVFRPDGSVLVVPK
(based on Phosphitispora fastidiosa sequence)

Other examples of amino acid residues that can be mutated within a facilitating domain described herein to reduce intrinsic cytidine deaminase activity include, without limitation, the amino acid residue that is immediately adjacent on the C-terminal side of the glutamic acid residue at (or predicted to be at) the active side of the intrinsic cytidine deaminase.

In some cases, a chimeric polypeptide provided herein can be designed to include a nucleic acid interacting domain, a facilitating domain having a mutation that reduces or eliminates an intrinsic cytidine deaminase activity of the facilitating domain, and an extrinsic base editing domain. Without being bound by any particular mechanism of action, the nucleic acid binding domain can target the chimeric polypeptide provided herein to a selected nucleotide sequence (e.g., a mitochondrial DNA sequence or a nuclear genomic DNA sequence), and the extrinsic base editing domain can generate a single base edit (e.g., a single C-to-T mutation, or a single A-to-G mutation) at or near the targeted sequence. Any appropriate extrinsic base editing domain can be included in such chimeric polypeptides. In some cases, for example, an adenosine deaminase domain can be included. A representative amino acid sequence for an adenosine deaminase domain is provided in SEQ ID NO:53:

(SEQ ID NO: 53)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNSKRGAAGSLMNVLNYPGMNHRVEITEGILADECAALLCDF
YRMPRQVFNAQKKAQSSIN

In some cases, a chimeric polypeptide provided herein can be designed to include an extrinsic base editing domain that includes the amino acid sequence set forth in SEQ ID NO: 51, or an extrinsic base editing domain having an amino acid sequence that is at least 90 percent (e.g., at least 95 percent, at least 96 percent, at least 97 percent, at least 98 percent, or at least 99 percent identical, or 100 percent identical) to the amino acid sequence set forth in SEQ ID NO:53. In some cases, a chimeric polypeptide provided herein can be designed to include SEQ ID NO:53 with (a) 1, 2, 3, 4, 5, 6, 7, 8, 9, or 10 amino acid additions, (b) 1, 2, 3, 4, 5, 6, 7, 8, 9, or 10 amino acid subtractions, (c) 1, 2, 3, 4, 5, 6, 7, 8, 9, or 10 amino acid substitutions, or (d) combinations thereof. For example, an extrinsic base editing domain can have an amino acid sequence that includes one or more conservative substitutions as compared to the amino acid sequence set forth in SEQ ID NO: 53.

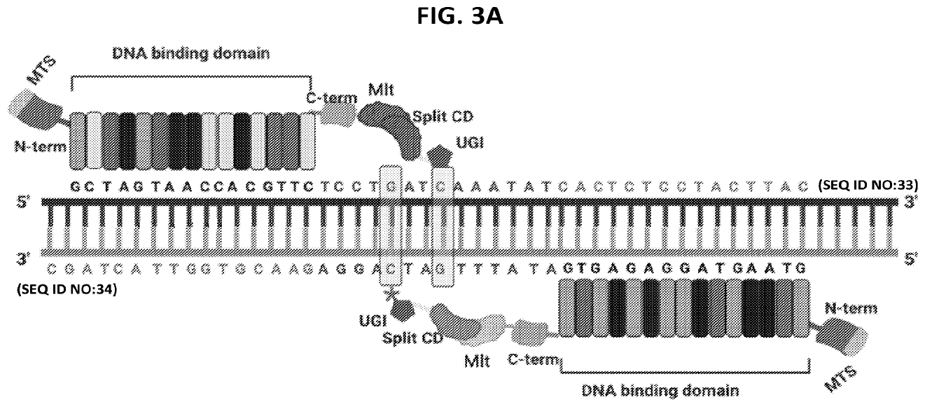

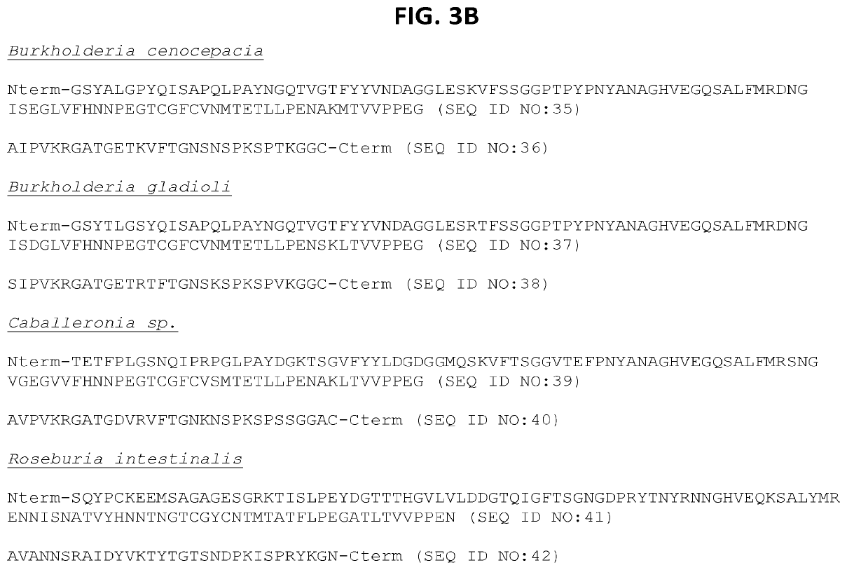

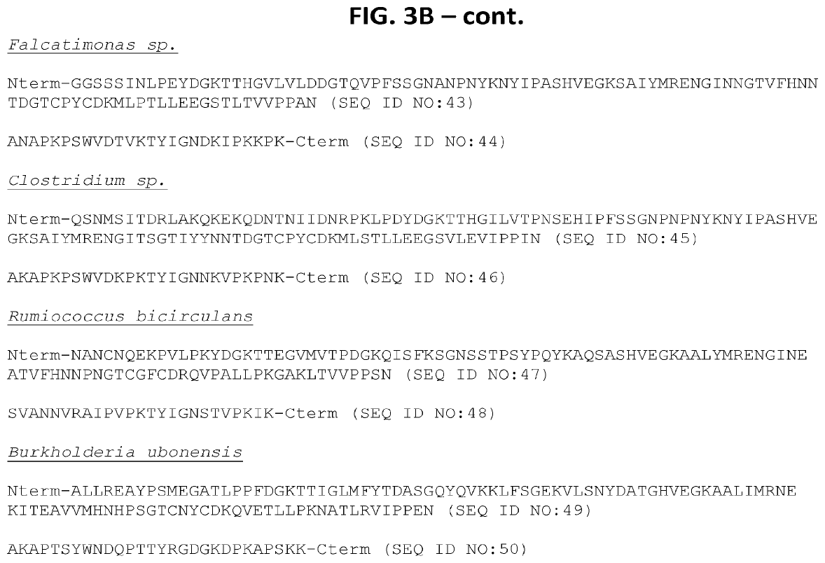

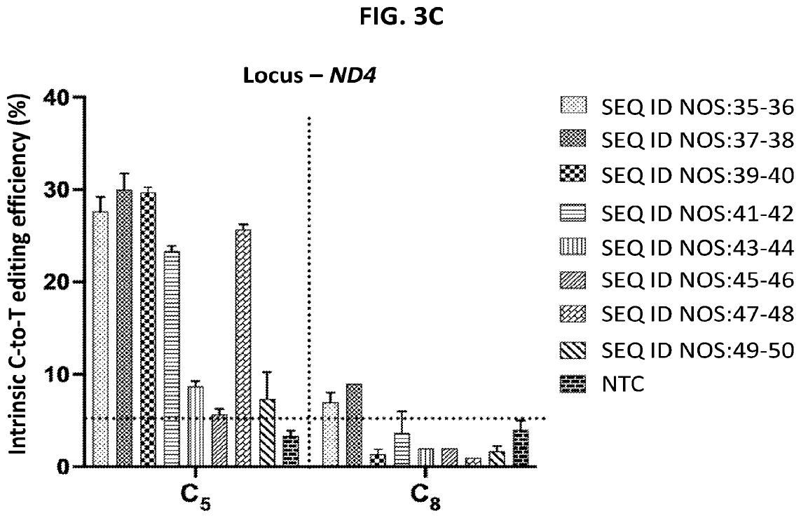

In some cases, a pair of chimeric polypeptides can be designed to act together to generate single base edits. For example, a first chimeric polypeptide can be designed to include a first nucleic acid interacting domain and the N-terminal portion of a facilitating domain, and a second chimeric polypeptide can be designed to include a second nucleic acid interacting domain and the C-terminal portion of a facilitating domain. In such cases, the first nucleic acid interacting domain can be targeted to a first nucleotide sequence, and the second nucleic acid interacting domain can be targeted to a second nucleotide sequence. On their own, neither chimeric polypeptide of the pair can generate a nucleic acid mutation (e.g., a single base edit) when bound to nucleic acid alone, but when each of the chimeric polypeptides of the pair bind to nearby locations of a target dsDNA (see, e.g., FIG. 3A) as a pair, base editing can occur. As illustrated in FIG. 3A, for example, first and second target nucleotide sequences can be selected such that when the first and second chimeric polypeptides interact with the first and second target sequences, respectively, the N-terminal and C-terminal portions of the facilitating domain are positioned so that base editing by the intrinsic cytidine deaminase can occur. Without being bound by any particular mechanism, separating the facilitating domain into two portions can reduce the potential cellular toxicity of the intrinsic cytidine deaminase when the chimeric polypeptides are introduced into a cell.

A facilitating domain described herein can be separated into any two appropriate portions. In some cases, for example, a facilitating domain described herein (e.g., a facilitating domain having the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO: 9, SEQ ID NO: 10, SEQ ID NO:11, SEQ ID NO:12, or SEQ ID NO: 13) can be split at any appropriate point to yield two portions of any appropriate length. In some cases, the N-terminal portion can be about 80 to about 130 amino acids in length (e.g., about 80 to about 90, about 90 to about 100, about 100 to about 110, about 110 to about 120, or about 120 to about 130 amino acids in length). In some cases, the N-terminal portion can be at least about 80 amino acids in length (e.g., at least about 80, at least about 85, at least about 90, at least about 95, at least about 100, at least about 105, at least about 110, at least about 115, at least about 120, or at least about 125 amino acids in length). In some cases, the C-terminal portion can be about 10 to about 40 amino acids in length (e.g., about 10 to about 20, about 15 to about 25, about 20 to about 30, about 25 to about 35, or about 30 to about 40 amino acids in length). In some cases, the C-terminal portion can be at least about 10 amino acids in length (e.g., at least about 10, at least about 15, at least about 20, at least about 25, or at least about 30 amino acids in length).

Representative examples of amino acid sequences for N-terminal and C-terminal portions of the facilitating domains set forth in SEQ ID NOS: 1-8 are set forth in SEQ ID NOS: 35-50 (FIG. 3B). Additional representative examples of amino acid sequences for N-terminal and C-terminal portions of the facilitating domains set forth in SEQ ID NOS: 9-13 are set forth in SEQ ID NOS: 103-112:

Representative Treponema sp. N-terminal portion:
(SEQ ID NO: 103)
SNNEKKKNANKQKIELPPYDGKTTYGVLILDDGKQYSFNSGKPAPIYRN
YIPASHVEGKAAIYMRENKIQSGTVYHNNTDGTCPYCDKMLPTLLEKDS
TLKVVPPQN
Representative Treponema sp. C-terminal portion:
(SEQ ID NO: 104)
ATSSKKGWITNEKIYIGNDKIPKTAR
Representative P. guartelaensis N-terminal
portion:
(SEQ ID NO: 105)
REDALLREQFPSMDAVTLPPFDGKTTIGYMFYTDANGQYHVRKLYSGGK
VLSNYDSSGHVEGMAALIMRKGRITEAVVMHNHPSGTCHYCNGQVETLL
PKNAKLKVIPPAN
Representative P. guartelaensis C-terminal
portion:
(SEQ ID NO: 106)
AKAPTKYWYDQPVDYLGNSNDPKPPS
Representative C. bacterium N-terminal portion:
(SEQ ID NO: 107)
SGFYQPANIPSSEVVLPEFDGKTTYGELRTPDGKSIPLQSGDPDPQYSN
YVSSSHVEGKAAQYMRENGIEQATVYHNNANGTCGYCDKMLPTLLPDGS
ELTVIPPAS
Representative C. bacterium C-terminal portion:
(SEQ ID NO: 108)
AVPNNPQAVAAPKTYTGNSAVPKTNPRFK
Representative P. violaceinigra N-terminal
portion:
(SEQ ID NO: 109)
GTNAAGSSGKNVRMPRDYASELPEYDGKTTHGVLVTNEGKVIQLRSGGK
EEPYTGYKAVSASHVEGKAAIWIRENGSSGGTVYHNNTTGTCGYCNSQV
KALLPEGVELKIVPPTN
Representative P. violaceinigra C-terminal
portion:
(SEQ ID NO: 110)
AVAKNAQARAVPTINVGNGTQPGRKQK
Representative P. fastidiosa N-terminal portion:
(SEQ ID NO: 111)
SRPRTPEDESIAAAIAERGKMTPAPKGKTSASVEGNSTESGWTTGPRAA
ENTEAVMELSKKMGHDLQPNKLLDQGKPGRYHASHAEKQAAVAAPNKPI
AVSAPMCDNCRRFFRALARYTGKPQT
Representative P. fastidiosa C-terminal portion:
(SEQ ID NO: 112)
VAEPRAVWVFRPDGSVLVVPK

In some cases, a chimeric polypeptide provided herein can include a nucleic acid interacting domain and/or a facilitating domain and/or an extrinsic base editing domain having one or more amino acid substitutions relative to a reference amino acid sequence (e.g., relative to any of SEQ ID NOs: 2-8, 9-13, 26-32, 37-50, 51, 52, 53, 98-102, and 103-112), where the one or more substituted amino acids are conservative substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of useful conservative substitutions can include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some cases, a chimeric polypeptide provided herein can include one or more non-conservative substitutions. Non-conservative substitutions typically entail exchanging a member of one of the classes described above for a member of another class. Such production can be desirable to provide large quantities or alternative embodiments of such compounds. Whether an amino acid change results in a functional polypeptide can readily be determined by assaying the specific activity of the polypeptide using, for example, methods disclosed herein.

In some cases, a chimeric polypeptide provided herein can include a mitochondrial targeting sequence (MTS) that can direct the chimeric polypeptide to a mitochondrion when the chimeric polypeptide is expressed in a cell. A chimeric polypeptide provided herein can be designed to have one or more MTS(s) located at any appropriate position. For example, a MTS can be present at the N-terminus of a chimeric polypeptide provided herein. Any appropriate MTS can be used. Non-limiting examples of an MTS that can be used include an isocitrate dehydrogenase 2 MTS, a human COX8A MTS, or a human SOD2 MTS. A representative COX8A MTS amino acid sequence is MASVLTPLLLRGLTGSARRLPVPRAKIHSL (SEQ ID NO:94), and a representative SOD2 MTC amino acid sequence is MALSRAVCGTSRQLAPVLGYL GSRQKHSLPD (SEQ ID NO:95). Other useful MTSs that can be used as described herein are described elsewhere (see, for example, Claros and Vincens, Eur. J. Biochem., 241:779-786, (1996)).

In some cases, a chimeric polypeptide provided herein can further include one or more linker sequences. For example, a chimeric polypeptide provided herein can include a linker sequence between the nucleic acid interacting domain and the facilitating domain. When a chimeric polypeptide provided herein includes an extrinsic base editing domain, the chimeric polypeptide can include a linker sequence between the nucleic acid interacting domain and the facilitating domain, and/or a linker sequence between the facilitating domain and the extrinsic base editing domain. In some cases, when a chimeric polypeptide provided herein includes a MTS, the chimeric polypeptide also can include a linker sequence between the MTS and the adjacent domain (e.g., the nucleic acid interacting domain). When two or more linker sequences are present, they can have the same amino acid sequence or they can have different amino acid sequences. The linker(s) can have any appropriate length. For example, a linker can be about 2 to about 10 amino acids in length, about 5 to about 15 amino acids in length, or about 10 to about 30 amino acids in length. A linker can have any appropriate sequence. Representative examples of linker sequences include, without limitation, SR, LVGS, SGGS, and SGSETPGTSESATPES (SEQ ID NO:96).

In some cases, a chimeric polypeptide provided herein designed to include cytosine deaminase activity (or each member of a pair of chimeric polypeptides provided herein designed to include cytosine deaminase activity when brought together) can further include one or more inhibitor of uracil DNA glycosylase domains (UGI domains) to inhibit base excision repair that changes a uracil back to a cytosine. An example of a UGI domain that can be incorporated into a chimeric polypeptide provided herein includes, without limitation, the UGI have the following amino acid sequence: TNLSDIIEKETGKQLVIQESILMLPEEVEEVIGNKPESDIL VHTAYDESTDENVMLL TSDAPEYKPWALVIQDSNGENKIKML (SEQ ID NO:97). In some cases, a chimeric polypeptide provided herein can be designed to include a nucleic acid interacting domain, a facilitating domain lacking intrinsic cytosine deaminase activity, an extrinsic base editing domain, one or more optional linker sequences, and one or more optional MTSs, in any appropriate order. By way of example, a chimeric polypeptide provided herein can include, in order from N-terminus to C-terminus, a MTS, a nucleic acid interacting domain, a linker, a facilitating domain lacking intrinsic cytosine deaminase activity, a second linker, and an extrinsic base editing domain (e.g., an extrinsic adenosine deaminase domain). In some cases, a chimeric polypeptide provided herein can be designed to include a nucleic acid interacting domain, a facilitating domain having an intrinsic base editing domain, one or more optional linker sequences, and one or more optional MTSs, in any appropriate order. By way of example, a chimeric polypeptide provided herein can include, in order from N-terminus to C-terminus, a MTS, a nucleic acid interacting domain, a linker, and a facilitating domain.

In some cases, a pair of chimeric polypeptides provided herein can be designed with a first one of the chimeric polypeptides of the pair including a first nucleic acid interacting domain, a first facilitating domain having a first portion of an intrinsic base editing domain, one or more optional linker sequences, and one or more optional MTSs, in any appropriate order, and a second one of the chimeric polypeptides of the pair including a second nucleic acid interacting domain, a second facilitating domain having a second portion of an intrinsic base editing domain, one or more optional linker sequences, and one or more optional MTSs, in any appropriate order. By way of example, a first one of a pair of chimeric polypeptides provided herein can include, in order from N-terminus to C-terminus, a MTS, a first nucleic acid interacting domain, a linker, and a first facilitating domain having a first portion of an intrinsic base editing domain, and a second one of the pair of chimeric polypeptides provided herein can include, in order from N-terminus to C-terminus, a MTS, a second nucleic acid interacting domain, a linker, and a second facilitating domain having a second portion of an intrinsic base editing domain. When the first and second portions of the intrinsic base editing domain are brought together, they can form a functional intrinsic base editing domain.

This document also provides nucleic acid molecules encoding a chimeric polypeptide provided herein. The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

In some cases, this document provides isolated nucleic acid molecules encoding a chimeric polypeptide provided herein that includes a nucleic acid interacting domain, a facilitating domain, and an extrinsic base editing domain, with or without a MTS and with or without one or more linker sequences. In some cases, this document provides isolated nucleic acid molecules encoding a chimeric polypeptide provided herein that includes a nucleic acid interacting domain and a portion of a facilitating domain (e.g., an N-terminal portion of a facilitating domain or a C-terminal portion of a facilitating domain), with or without a MTS and with or without one or more linkers.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

Isolated nucleic acid molecules can be produced using any appropriate techniques, including, without limitation, molecular cloning and chemical nucleic acid synthesis techniques. For example, polymerase chain reaction (PCR) techniques can be used to obtain an isolated nucleic acid containing nucleotide sequence that encodes a chimeric polypeptide provided herein. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication, or nucleic acid sequence-based amplification also can be used to obtain isolated nucleic acids. See, for example, Lewis, Genetic Engineering News, 12:1 (1992); Guatelli et al., Proc. Natl. Acad. Sci. USA, 87:1874-1878 (1990); and Weiss Science, 254:1292 (1991).

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

This document also provides vectors containing a nucleic acid encoding a chimeric polypeptide described herein. A “vector” is a replicon, such as a plasmid, phage, or cosmid, into which another DNA segment may be inserted so as to bring about the replication of the inserted segment. An “expression vector” is a vector that includes one or more expression control sequences, and an “expression control sequence” is a DNA sequence that controls and regulates the transcription and/or translation of another DNA sequence.

In an expression vector, a nucleic acid (e.g., a nucleic acid encoding a chimeric polypeptide provided herein) can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 to 500 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence. Expression vectors thus can be useful to produce antibodies as well as other multivalent molecules.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen/Life Technologies (Carlsbad, CA).

Also provided herein are methods that include using a chimeric polypeptide described herein to modify nucleic acid (e.g., mitochondrial DNA or nuclear genomic DNA) within a cell (e.g., a eukaryotic cell). For example, methods provided herein can be used to generate single base mutations within the mitochondrial or nuclear genomic DNA of a cell. The methods can include introducing into a cell (e.g., a cell in culture in vitro, or a cell in vivo within a eukaryotic organism) one or more chimeric polypeptides provided herein. In some cases, a method provided herein can include introducing, into a cell, a chimeric polypeptide provided herein that includes a nucleic acid interacting domain, a facilitating domain having a mutated intrinsic base editing domain (e.g., a non-functional cytidine deaminase domain), and an extrinsic base editing domain (e.g., an adenosine deaminase domain). In some cases, a method provided herein can include introducing, into a cell, nucleic acid encoding a chimeric polypeptide provided herein that includes a nucleic acid interacting domain, a facilitating domain having a mutated intrinsic base editing domain (e.g., a non-functional cytidine deaminase domain), and an extrinsic base editing domain (e.g., an adenosine deaminase domain). When such a chimeric polypeptide is introduced into a cell, or when nucleic acid encoding such a chimeric polypeptide is introduced into and subsequently expressed in a cell, the chimeric polypeptide can interact with its target sequence via the nucleic acid interacting domain, and a base within the sequence adjacent to the target sequence (e.g., a base within about 2 to about 30 nucleotides, about 5 to about 10 nucleotides, about 10 to about 15 nucleotides, or about 15 to about 20 nucleotides of the target sequence) can be modified (e.g., deaminated) by the extrinsic base editing domain.

In some cases, a method provided herein can include introducing, into a cell, a pair of chimeric polypeptides provided herein, where the first member of the pair includes a first nucleic acid interacting domain targeted to a first selected nucleotide sequence and a first facilitating domain that includes a first portion (e.g., an N-terminal portion) of an intrinsic cytidine deaminase, and where the second member of the pair includes a second nucleic acid interacting domain targeted to a second selected nucleotide sequence and a second facilitating domain that includes a second portion (e.g., a C-terminal portion) of an intrinsic cytidine deaminase. In some cases, a method provided herein can include introducing, into a cell, nucleic acid encoding a pair of chimeric polypeptides provided herein, where the first member of the pair includes a first nucleic acid interacting domain targeted to a first selected nucleotide sequence and a first facilitating domain that includes a first portion (e.g., an N-terminal portion) of an intrinsic cytidine deaminase, and where the second member of the pair includes a second nucleic acid interacting domain targeted to a second selected nucleotide sequence and a second facilitating domain that includes a second portion (e.g., a C-terminal portion) of an intrinsic cytidine deaminase. When such a pair of chimeric polypeptides provided herein are used, the first and second nucleic acid interacting domains can be targeted to sequences such that when the first nucleic acid interacting domain interacts with the first target sequence and the second nucleic acid interacting domain interacts with the second target sequence, the first and second portions of the cytidine deaminase are in proximity to one another and can generate a single base C-to-T mutation. The first and second target sequences can be selected such that they are separated by a spacer sequence within which the single base mutation is to be made. The spacer sequence can have any appropriate length. For example, the spacer can be about 10 to about 20 (e.g., 10 to 12, 10 to 15, 12 to 14, 14 to 16, 15 to 20, 16 to 18, or 18 to 20) nucleotides in length. In some cases, the first and second target sequences can be on opposite strands of DNA (e.g., as illustrated in FIG. 3A).

Any appropriate cells can be used in the methods provided herein. For example, vertebrate cells (e.g., zebrafish cells, mouse cells, rat cells, rabbit cells, sheep cells, pig cells, cow cells, horse cells, dog cells, or human cells) can be used. The methods can include introducing into a cell one or more (e.g., one, two, three, four, or more than four) chimeric polypeptides, either by introducing one or more chimeric polypeptides or by introducing nucleic acid encoding the one or more chimeric polypeptides, as described herein. In some cases, the methods provided herein can include introducing chimeric polypeptides (or nucleic acid molecules encoding chimeric polypeptides) targeted to different sequences.

Any suitable method can be used to introduce a nucleic acid molecule encoding one or more chimeric polypeptides into a cell in vivo or in vitro. For example, calcium phosphate precipitation, electroporation, lipofection, microinjection, nanoparticle-based delivery, and viral-mediated nucleic acid transfer are methods that can be used to introduce one or more isolated nucleic acid molecules (e.g., one or more isolated nucleic acids encoding one or more chimeric polypeptides provided herein) into a cell. The cell can be cultured such that the one or more chimeric polypeptides can interact with cellular nucleic acid (e.g., genomic nucleic acid and/or mitochondrial nucleic acid) and generate a mutation (e.g., a single base C-to-T mutation or a single base A-to-G mutation).

The cell, or progeny thereof, can be assessed using, for example, PCR and sequencing techniques to determine whether a mutation has been generated at or near a sequence targeted by the nucleic acid interacting domain of a chimeric polypeptide provided herein. In some cases, when mitochondrial DNA is targeted by one or more chimeric polypeptides, software such as EditR can be used to assess mitochondrial heteroplasmy, and to estimate the percentage of edits in the target loci. In some cases, when the transformed cells are within an organism (e.g., an embryo), the organism can be allowed to develop, and cells within the resulting organism can be assessed to determine whether the mutation(s) have been maintained.

### EXEMPLARY EMBODIMENTS

Embodiment 1 is chimeric polypeptide comprising a nucleic acid interacting domain and a facilitating domain, wherein said facilitating domain comprises:
an amino acid sequence (i) that comprises the amino acid sequence set forth in SEQ ID NO:2, (ii) that is at least 92 percent identical to the amino acid sequence set forth in SEQ ID NO:2, (iii) that comprises an N-terminal portion of the amino acid sequence set forth in SEQ ID NO:2 that is at least 100 amino acid residues in length, (iv) that is at least 92 percent identical to said N-terminal portion, (v) that comprises a C-terminal portion of the amino acid sequence set forth in SEQ ID NO:2 that is at least 25 amino acid residues in length, or (vi) that is at least 92 percent identical to said C-terminal portion;
an amino acid sequence (i) that comprises the amino acid sequence set forth in SEQ ID NO:3, (ii) that is at least 85 percent identical to the amino acid sequence set forth in SEQ ID NO:3, (iii) that comprises an N-terminal portion of the amino acid sequence set forth in SEQ ID NO:3 that is at least 100 amino acid residues in length, (iv) that is at least 85 percent identical to said N-terminal portion, (v) that comprises a C-terminal portion of the amino acid sequence set forth in SEQ ID NO:3 that is at least 25 amino acid residues in length, or (vi) that is at least 85 percent identical to said C-terminal portion;
an amino acid sequence (i) that comprises the amino acid sequence set forth in SEQ ID NO:4, (ii) that is at least 85 percent identical to the amino acid sequence set forth in SEQ ID NO:4, (iii) that comprises an N-terminal portion of the amino acid sequence set forth in SEQ ID NO:4 that is at least 105 amino acid residues in length, (iv) that is at least 85 percent identical to said N-terminal portion, (v) that comprises a C-terminal portion of the amino acid sequence set forth in SEQ ID NO:4 that is at least 25 amino acid residues in length, or (vi) that is at least 85 percent identical to said C-terminal portion;
an amino acid sequence (i) that comprises the amino acid sequence set forth in SEQ ID NO:5, (ii) that is at least 85 percent identical to the amino acid sequence set forth in SEQ ID NO:5, (iii) that comprises an N-terminal portion of the amino acid sequence set forth in SEQ ID NO:5 that is at least 90 amino acid residues in length, (iv) that is at least 85 percent identical to said N-terminal portion, (v) that comprises a C-terminal portion of the amino acid sequence set forth in SEQ ID NO:5 that is at least 20 amino acid residues in length, or (vi) that is at least 85 percent identical to said C-terminal portion;
an amino acid sequence (i) that comprises the amino acid sequence set forth in SEQ ID NO:6, (ii) that is at least 85 percent identical to the amino acid sequence set forth in SEQ ID NO:6, (iii) that comprises an N-terminal portion of the amino acid sequence set forth in SEQ ID NO:6 that is at least 110 amino acid residues in length, (iv) that is at least 85 percent identical to said N-terminal portion, (v) that comprises a C-terminal portion of the amino acid sequence set forth in SEQ ID NO:6 that is at least 20 amino acid residues in length, or (vi) that is at least 85 percent identical to said C-terminal portion;
an amino acid sequence (i) that comprises the amino acid sequence set forth in SEQ ID NO:7, (ii) that is at least 85 percent identical to the amino acid sequence set forth in SEQ ID NO:7, (iii) that comprises an N-terminal portion of the amino acid sequence set forth in SEQ ID NO:7 that is at least 100 amino acid residues in length, (iv) that is at least 85 percent identical to said N-terminal portion, (v) that comprises a C-terminal portion of the amino acid sequence set forth in SEQ ID NO:7 that is at least 20 amino acid residues in length, or (vi) that is at least 85 percent identical to said C-terminal portion;
an amino acid sequence (i) that comprises the amino acid sequence set forth in SEQ ID NO:8, (ii) that is at least 85 percent identical to the amino acid sequence set forth in SEQ ID NO:8, (iii) that comprises an N-terminal portion of the amino acid sequence set forth in SEQ ID NO:8 that is at least 100 amino acid residues in length, (iv) that is at least 85 percent identical to said N-terminal portion, (v) that comprises a C-terminal portion of the amino acid sequence set forth in SEQ ID NO:8 that is at least 20 amino acid residues in length, or (vi) that is at least 85 percent identical to said C-terminal portion;
an amino acid sequence (i) that comprises the amino acid sequence set forth in SEQ ID NO:9, (ii) that is at least 90 percent identical to the amino acid sequence set forth in SEQ ID NO:9, (iii) that comprises an N-terminal portion of the amino acid sequence set forth in SEQ ID NO:9 that is at least 100 amino acid residues in length, (iv) that is at least 93% identical to said N-terminal portion, (v) that comprises a C-terminal portion of the amino acid sequence set forth in SEQ ID NO:9 that is at least 23 amino acid residues in length, or (vi) that is at least 88% identical to said C-terminal portion;
an amino acid sequence (i) that comprises the amino acid sequence set forth in SEQ ID NO:10, (ii) that is at least 90 percent identical to the amino acid sequence set forth in SEQ ID NO:10, (iii) that comprises an N-terminal portion of the amino acid sequence set forth in SEQ ID NO: 10 that is at least 105 amino acid residues in length, (iv) that is at least 94% identical to said N-terminal portion, (v) that comprises a C-terminal portion of the amino acid sequence set forth in SEQ ID NO:10 that is at least 23 amino acid residues in length, or (vi) that is at least 88% identical to said C-terminal portion;
an amino acid sequence (i) that comprises the amino acid sequence set forth in SEQ ID NO:11, (ii) that is at least 90 percent identical to the amino acid sequence set forth in SEQ ID NO:11, (iii) that comprises an N-terminal portion of the amino acid sequence set forth in SEQ ID NO: 11 that is at least 100 amino acid residues in length, (iv) that is at least 93% identical to said N-terminal portion, (v) that comprises a C-terminal portion of the amino acid sequence set forth in SEQ ID NO:11 that is at least 25 amino acid residues in length, or (vi) that is at least 86% identical to said C-terminal portion;
an amino acid sequence (i) that comprises the amino acid sequence set forth in SEQ ID NO:12, (ii) that is at least 90 percent identical to the amino acid sequence set forth in SEQ ID NO:12, (iii) that comprises an N-terminal portion of the amino acid sequence set forth in SEQ ID NO: 12 that is at least 108 amino acid residues in length, (iv) that is at least 93% identical to said N-terminal portion, (v) that comprises a C-terminal portion of the amino acid sequence set forth in SEQ ID NO:12 that is at least 23 amino acid residues in length, or (vi) that is at least 85% identical to said C-terminal portion; or
an amino acid sequence (i) that comprises the amino acid sequence set forth in SEQ ID NO:13, (ii) that is at least 90 percent identical to the amino acid sequence set forth in SEQ ID NO: 13, (iii) that comprises an N-terminal portion of the amino acid sequence set forth in SEQ ID NO:13 that is at least 116 amino acid residues in length, (iv) that is at least 93% identical to said N-terminal portion, (v) that comprises a C-terminal portion of the amino acid sequence set forth in SEQ ID NO:13 that is at least 18 amino acid residues in length, or (vi) that is at least 85% identical to said C-terminal portion.

Embodiment 2 is the chimeric polypeptide of embodiment 1, wherein said nucleic acid interacting domain is N-terminal to said facilitating domain.

Embodiment 3 is the chimeric polypeptide of embodiment 1 or embodiment 2, wherein said nucleic acid interacting domain comprises a transcription activator-like effector (TALE) DNA binding domain.

Embodiment 4 is the chimeric polypeptide of embodiment 1 or embodiment 2, wherein said nucleic acid interacting domain comprises a zinc finger DNA binding domain.

Embodiment 5 is the chimeric polypeptide of embodiment 1 or embodiment 2, wherein said nucleic acid interacting domain comprises CRISPR/Cas DNA binding components.

Embodiment 6 is the chimeric polypeptide of any one of embodiments 1 to 5, further comprising a linker between said nucleic acid interacting domain and said facilitating domain.

Embodiment 7 is the chimeric polypeptide of any one of embodiments 1 to 6, further comprising a mitochondrial targeting sequence (MTS).

Embodiment 8 is the chimeric polypeptide of embodiment 7, wherein said MTS is an isocitrate dehydrogenase 2 MTS, a human COX8A MTS, or a human SOD2 MTS.

Embodiment 9 is the chimeric polypeptide of embodiment 7 or embodiment 8, wherein said MTS is at the N-terminus of said chimeric polypeptide.

Embodiment 10 is the chimeric polypeptide of embodiment 9, wherein said chimeric polypeptide comprises, in order from N-terminus to C-terminus, said MTS, said nucleic acid interacting domain, and said facilitating domain, and wherein said chimeric polypeptide further comprises a linker between said MTS and said nucleic acid interacting domain.

Embodiment 11 is the chimeric polypeptide of any one of embodiments 1 to 10, wherein said facilitating domain comprises intrinsic cytosine deaminase activity.

Embodiment 12 is the chimeric polypeptide of any one of embodiments 1 to 10, wherein said facilitating domain lacks cytosine deaminase activity, and wherein said chimeric polypeptide further comprises an extrinsic deaminase domain.

Embodiment 13 is the chimeric polypeptide of embodiment 12, wherein said extrinsic deaminase domain is an adenosine deaminase domain.

Embodiment 14 is the chimeric polypeptide of embodiment 13, wherein said adenosine deaminase domain comprises an amino acid sequence at least 85 percent identical to the amino acid sequence set forth in SEQ ID NO:53.

Embodiment 15 is the chimeric polypeptide of embodiment 12, wherein said extrinsic deaminase domain is a cytosine deaminase domain.

Embodiment 16 is the chimeric polypeptide of any one of embodiments 12 to 15, wherein said chimeric polypeptide comprises, in order from N-terminus to C-terminus, said nucleic acid interacting domain, said facilitating domain, and said extrinsic deaminase domain.

Embodiment 17 is the chimeric polypeptide of embodiment 16, further comprising a MTS at the N-terminus of said chimeric polypeptide, a first linker between said nucleic acid interacting domain and said facilitating domain, and a second linker between said facilitating domain and said extrinsic deaminase domain.

Embodiment 18 is the chimeric polypeptide of any one of embodiments 1 to 10, wherein said facilitating domain comprises:
said N-terminal portion of the amino acid sequence set forth in SEQ ID NO:2 that is at least 100 amino acid residues in length, or said amino acid sequence that is at least 92 percent identical to said N-terminal portion;
said N-terminal portion of the amino acid sequence set forth in SEQ ID NO:3 that is at least 100 amino acid residues in length, or said amino acid sequence that is at least 85 percent identical to said N-terminal portion;
said N-terminal portion of the amino acid sequence set forth in SEQ ID NO:4 that is at least 105 amino acid residues in length, or said amino acid sequence that is at least 85 percent identical to said N-terminal portion;
said N-terminal portion of the amino acid sequence set forth in SEQ ID NO:5 that is at least 90 amino acid residues in length, or said amino acid sequence that is at least 85 percent identical to said N-terminal portion;
said N-terminal portion of the amino acid sequence set forth in SEQ ID NO:6 that is at least 110 amino acid residues in length, or said amino acid sequence that is at least 85 percent identical to said N-terminal portion;
said N-terminal portion of the amino acid sequence set forth in SEQ ID NO:7 that is at least 100 amino acid residues in length, or said amino acid sequence that is at least 85 percent identical to said N-terminal portion; or
said N-terminal portion of the amino acid sequence set forth in SEQ ID NO:8 that is at least 100 amino acid residues in length, or said amino acid sequence that is at least 85 percent identical to said N-terminal portion.

Embodiment 19 is the chimeric polypeptide of any one of embodiments 1 to 10, wherein said facilitating domain comprises:
said C-terminal portion of the amino acid sequence set forth in SEQ ID NO:2 that is at least 25 amino acid residues in length, or said amino acid sequence that is at least 92 percent identical to said C-terminal portion;
said C-terminal portion of the amino acid sequence set forth in SEQ ID NO:3 that is at least 25 amino acid residues in length, or said amino acid sequence that is at least 85 percent identical to said C-terminal portion;
said C-terminal portion of the amino acid sequence set forth in SEQ ID NO:4 that is at least 25 amino acid residues in length, or said amino acid sequence that is at least 85 percent identical to said C-terminal portion;
said C-terminal portion of the amino acid sequence set forth in SEQ ID NO:5 that is at least 20 amino acid residues in length, or said amino acid sequence that is at least 85 percent identical to said C-terminal portion;
said C-terminal portion of the amino acid sequence set forth in SEQ ID NO:6 that is at least 20 amino acid residues in length, or said amino acid sequence that is at least 85 percent identical to said C-terminal portion;
said C-terminal portion of the amino acid sequence set forth in SEQ ID NO:7 that is at least 20 amino acid residues in length, or said amino acid sequence that is at least 85 percent identical to said C-terminal portion; or
said C-terminal portion of the amino acid sequence set forth in SEQ ID NO:8 that is at least 20 amino acid residues in length, or said amino acid sequence that is at least 85 percent identical to said C-terminal portion.

Embodiment 20 is a nucleic acid comprising a nucleotide sequence encoding the chimeric polypeptide of any one of embodiments 1 to 19.

Embodiment 21 is a vector comprising the nucleic acid of embodiment 20.

Embodiment 22 is a cell comprising the nucleic acid of embodiment 19 or the vector of embodiment 20.

Embodiment 23 is a method for generating a mutation within mitochondrial or nuclear genomic DNA of a cell, comprising (a) introducing, into the cell, a chimeric polypeptide comprising a nucleic acid interacting domain and a facilitating domain, and (b) incubating said cell such that said chimeric polypeptide generates a mutation within said mitochondrial or said nucleic genomic DNA, wherein said facilitating domain comprises:
(1) an amino acid sequence (i) that comprises the amino acid sequence set forth in SEQ ID NO:2, (ii) that is at least 92 percent identical to the amino acid sequence set forth in SEQ ID NO:2, (iii) that comprises an N-terminal portion of the amino acid sequence set forth in SEQ ID NO:2 that is at least 100 amino acid residues in length, (iv) that is at least 92 percent identical to said N-terminal portion, (v) that comprises a C-terminal portion of the amino acid sequence set forth in SEQ ID NO:2 that is at least 25 amino acid residues in length, or (vi) that is at least 92 percent identical to said C-terminal portion;
(2) an amino acid sequence (i) that comprises the amino acid sequence set forth in SEQ ID NO:3, (ii) that is at least 85 percent identical to the amino acid sequence set forth in SEQ ID NO:3, (iii) that comprises an N-terminal portion of the amino acid sequence set forth in SEQ ID NO:3 that is at least 100 amino acid residues in length, (iv) that is at least 85 percent identical to said N-terminal portion, (v) that comprises a C-terminal portion of the amino acid sequence set forth in SEQ ID NO:3 that is at least 25 amino acid residues in length, or (vi) that is at least 85 percent identical to said C-terminal portion;
(3) an amino acid sequence (i) that comprises the amino acid sequence set forth in SEQ ID NO:4, (ii) that is at least 85 percent identical to the amino acid sequence set forth in SEQ ID NO:4, (iii) that comprises an N-terminal portion of the amino acid sequence set forth in SEQ ID NO:4 that is at least 105 amino acid residues in length, (iv) that is at least 85 percent identical to said N-terminal portion, (v) that comprises a C-terminal portion of the amino acid sequence set forth in SEQ ID NO:4 that is at least 25 amino acid residues in length, or (vi) that is at least 85 percent identical to said C-terminal portion;
(4) an amino acid sequence (i) that comprises the amino acid sequence set forth in SEQ ID NO:5, (ii) that is at least 85 percent identical to the amino acid sequence set forth in SEQ ID NO:5, (iii) that comprises an N-terminal portion of the amino acid sequence set forth in SEQ ID NO:5 that is at least 90 amino acid residues in length, (iv) that is at least 85 percent identical to said N-terminal portion, (v) that comprises a C-terminal portion of the amino acid sequence set forth in SEQ ID NO:5 that is at least 20 amino acid residues in length, or (vi) that is at least 85 percent identical to said C-terminal portion;
(5) an amino acid sequence (i) that comprises the amino acid sequence set forth in SEQ ID NO:6, (ii) that is at least 85 percent identical to the amino acid sequence set forth in SEQ ID NO:6, (iii) that comprises an N-terminal portion of the amino acid sequence set forth in SEQ ID NO:6 that is at least 110 amino acid residues in length, (iv) that is at least 85 percent identical to said N-terminal portion, (v) that comprises a C-terminal portion of the amino acid sequence set forth in SEQ ID NO:6 that is at least 20 amino acid residues in length, or (vi) that is at least 85 percent identical to said C-terminal portion;
(6) an amino acid sequence (i) that comprises the amino acid sequence set forth in SEQ ID NO:7, (ii) that is at least 85 percent identical to the amino acid sequence set forth in SEQ ID NO:7, (iii) that comprises an N-terminal portion of the amino acid sequence set forth in SEQ ID NO:7 that is at least 100 amino acid residues in length, (iv) that is at least 85 percent identical to said N-terminal portion, (v) that comprises a C-terminal portion of the amino acid sequence set forth in SEQ ID NO:7 that is at least 20 amino acid residues in length, or (vi) that is at least 85 percent identical to said C-terminal portion;
(7) an amino acid sequence (i) that comprises the amino acid sequence set forth in SEQ ID NO:8, (ii) that is at least 85 percent identical to the amino acid sequence set forth in SEQ ID NO:8, (iii) that comprises an N-terminal portion of the amino acid sequence set forth in SEQ ID NO:8 that is at least 100 amino acid residues in length, (iv) that is at least 85 percent identical to said N-terminal portion, (v) that comprises a C-terminal portion of the amino acid sequence set forth in SEQ ID NO:8 that is at least 20 amino acid residues in length, or (vi) that is at least 85 percent identical to said C-terminal portion;
(8) an amino acid sequence (i) that comprises the amino acid sequence set forth in SEQ ID NO:9, (ii) that is at least 90 percent identical to the amino acid sequence set forth in SEQ ID NO:9, (iii) that comprises an N-terminal portion of the amino acid sequence set forth in SEQ ID NO:9 that is at least 100 amino acid residues in length, (iv) that is at least 93% identical to said N-terminal portion, (v) that comprises a C-terminal portion of the amino acid sequence set forth in SEQ ID NO:9 that is at least 23 amino acid residues in length, or (vi) that is at least 88% identical to said C-terminal portion;
(9) an amino acid sequence (i) that comprises the amino acid sequence set forth in SEQ ID NO:10, (ii) that is at least 90 percent identical to the amino acid sequence set forth in SEQ ID NO:10, (iii) that comprises an N-terminal portion of the amino acid sequence set forth in SEQ ID NO:10 that is at least 105 amino acid residues in length, (iv) that is at least 94% identical to said N-terminal portion, (v) that comprises a C-terminal portion of the amino acid sequence set forth in SEQ ID NO: 10 that is at least 23 amino acid residues in length, or (vi) that is at least 88% identical to said C-terminal portion;
(10) an amino acid sequence (i) that comprises the amino acid sequence set forth in SEQ ID NO:11, (ii) that is at least 90 percent identical to the amino acid sequence set forth in SEQ ID NO:11, (iii) that comprises an N-terminal portion of the amino acid sequence set forth in SEQ ID NO:11 that is at least 100 amino acid residues in length, (iv) that is at least 93% identical to said N-terminal portion, (v) that comprises a C-terminal portion of the amino acid sequence set forth in SEQ ID NO:11 that is at least 25 amino acid residues in length, or (vi) that is at least 86% identical to said C-terminal portion;
(11) an amino acid sequence (i) that comprises the amino acid sequence set forth in SEQ ID NO:12, (ii) that is at least 90 percent identical to the amino acid sequence set forth in SEQ ID NO:12, (iii) that comprises an N-terminal portion of the amino acid sequence set forth in SEQ ID NO:12 that is at least 108 amino acid residues in length, (iv) that is at least 93% identical to said N-terminal portion, (v) that comprises a C-terminal portion of the amino acid sequence set forth in SEQ ID NO: 12 that is at least 23 amino acid residues in length, or (vi) that is at least 85% identical to said C-terminal portion; or
(12) an amino acid sequence (i) that comprises the amino acid sequence set forth in SEQ ID NO: 13, (ii) that is at least 90 percent identical to the amino acid sequence set forth in SEQ ID NO:13, (iii) that comprises an N-terminal portion of the amino acid sequence set forth in SEQ ID NO:13 that is at least 116 amino acid residues in length, (iv) that is at least 93% identical to said N-terminal portion, (v) that comprises a C-terminal portion of the amino acid sequence set forth in SEQ ID NO:13 that is at least 18 amino acid residues in length, or (vi) that is at least 85% identical to said C-terminal portion.

Embodiment 24 is the method of embodiment 23, wherein said mutation is a single base substitution.

Embodiment 25 is the method of embodiment 23 or embodiment 24, wherein said introducing comprises introducing nucleic acid encoding said chimeric polypeptide into said cell.

Embodiment 26 is the method of any one of embodiments 23 to 25, wherein said nucleic acid interacting domain is N-terminal to said facilitating domain.

Embodiment 27 is the method of any one of embodiments 23 to 26, wherein said nucleic acid interacting domain comprises a TALE DNA binding domain.

Embodiment 28 is the method of any one of embodiments 23 to 26, wherein said nucleic acid interacting domain comprises a zinc finger DNA binding domain.

Embodiment 29 is the method of any one of embodiments 23 to 26, wherein said nucleic acid interacting domain comprises CRISPR/Cas DNA binding components.

Embodiment 30 is the method of any one of embodiments 23 to 29, wherein said chimeric polypeptide further comprises a linker between said nucleic acid interacting domain and said facilitating domain.

Embodiment 31 is the method of any one of embodiments 23 to 30, wherein said chimeric polypeptide further comprises a MTS.

Embodiment 32 is the method of embodiment 31, wherein said MTS is at the N-terminus of said chimeric polypeptide.

Embodiment 33 is the chimeric polypeptide of embodiment 31 or embodiment 32, wherein said MTS is an isocitrate dehydrogenase 2 MTS, a human COX8A MTS, or a human SOD2 MTS.

Embodiment 34 is the method of any one of embodiments 31 to 33, wherein said chimeric polypeptide comprises, in order from N-terminus to C-terminus, said MTS, said nucleic acid interacting domain, and said facilitating domain, and wherein said chimeric polypeptide further comprises a linker between said MTS and said nucleic acid interacting domain.

Embodiment 35 is the method of any one of embodiments 23 to 34, wherein said facilitating domain comprises intrinsic cytosine deaminase activity.

Embodiment 36 is the method of any one of embodiments 23 to 34, wherein said facilitating domain lacks cytosine deaminase activity, and wherein said chimeric polypeptide further comprises an extrinsic deaminase domain.

Embodiment 37 is the method of embodiment 36, wherein said extrinsic deaminase domain is an adenosine deaminase domain.

Embodiment 38 is the method of embodiment 37, wherein said adenosine deaminase domain comprises an amino acid sequence at least 90 percent identical to the amino acid sequence set forth in SEQ ID NO:53.

Embodiment 39 is the method of embodiment 36, wherein said extrinsic deaminase domain is a cytosine deaminase domain.

Embodiment 40 is the method of any one of embodiments 36 to 39, wherein said chimeric polypeptide comprises, in order from N-terminus to C-terminus, said nucleic acid interacting domain, said facilitating domain, and said extrinsic deaminase domain.

Embodiment 41 is the method of embodiment 40, wherein said chimeric polypeptide further comprises a MTS at the N-terminus of said chimeric polypeptide, a first linker between said nucleic acid interacting domain and said facilitating domain, and a second linker between said facilitating domain and said extrinsic deaminase domain.

Embodiment 42 is the method of any one of embodiments 23 to 34, wherein said method comprises introducing a first chimeric polypeptide and a second chimeric polypeptide into said cell, wherein said facilitating domain of said first chimeric polypeptide comprises:
said N-terminal portion of the amino acid sequence set forth in SEQ ID NO:2 that is at least 100 amino acid residues in length, or said amino acid sequence that is at least 92 percent identical to said N-terminal portion;
said N-terminal portion of the amino acid sequence set forth in SEQ ID NO:3 that is at least 100 amino acid residues in length, or said amino acid sequence that is at least 85 percent identical to said N-terminal portion;
said N-terminal portion of the amino acid sequence set forth in SEQ ID NO:4 that is at least 105 amino acid residues in length, or said amino acid sequence that is at least 85 percent identical to said N-terminal portion;
said N-terminal portion of the amino acid sequence set forth in SEQ ID NO:5 that is at least 90 amino acid residues in length, or said amino acid sequence that is at least 85 percent identical to said N-terminal portion;
said N-terminal portion of the amino acid sequence set forth in SEQ ID NO:6 that is at least 110 amino acid residues in length, or said amino acid sequence that is at least 85 percent identical to said N-terminal portion;
said N-terminal portion of the amino acid sequence set forth in SEQ ID NO:7 that is at least 100 amino acid residues in length, or said amino acid sequence that is at least 85 percent identical to said N-terminal portion; or
said N-terminal portion of the amino acid sequence set forth in SEQ ID NO:8 that is at least 100 amino acid residues in length, or said amino acid sequence that is at least 85 percent identical to said N-terminal portion, and wherein said facilitating domain of said second chimeric polypeptide comprises:
said C-terminal portion of the amino acid sequence set forth in SEQ ID NO:2 that is at least 25 amino acid residues in length, or said amino acid sequence that is at least 92 percent identical to said C-terminal portion;
said C-terminal portion of the amino acid sequence set forth in SEQ ID NO:3 that is at least 25 amino acid residues in length, or said amino acid sequence that is at least 85 percent identical to said C-terminal portion;
said C-terminal portion of the amino acid sequence set forth in SEQ ID NO:4 that is at least 25 amino acid residues in length, or said amino acid sequence that is at least 85 percent identical to said C-terminal portion;
said C-terminal portion of the amino acid sequence set forth in SEQ ID NO:5 that is at least 20 amino acid residues in length, or said amino acid sequence that is at least 85 percent identical to said C-terminal portion;
said C-terminal portion of the amino acid sequence set forth in SEQ ID NO:6 that is at least 20 amino acid residues in length, or said amino acid sequence that is at least 85 percent identical to said C-terminal portion;
said C-terminal portion of the amino acid sequence set forth in SEQ ID NO:7 that is at least 20 amino acid residues in length, or said amino acid sequence that is at least 85 percent identical to said C-terminal portion; or
said C-terminal portion of the amino acid sequence set forth in SEQ ID NO:8 that is at least 20 amino acid residues in length, or said amino acid sequence that is at least 85 percent identical to said C-terminal portion.

Embodiment 43 is the method of embodiment 42, wherein said method comprises introducing a first chimeric polypeptide and a second chimeric polypeptide into said cell, and
wherein said facilitating domain of said first chimeric polypeptide comprises said N-terminal portion of the amino acid sequence set forth in SEQ ID NO:2 that is at least 100 amino acid residues in length, or said amino acid sequence that is at least 92 percent identical to said N-terminal portion, and wherein said facilitating domain of said second chimeric polypeptide comprises said C-terminal portion of the amino acid sequence set forth in SEQ ID NO:2 that is at least 25 amino acid residues in length, or said amino acid sequence that is at least 92 percent identical to said C-terminal portion; or
wherein said facilitating domain of said first chimeric polypeptide comprises said N-terminal portion of the amino acid sequence set forth in SEQ ID NO:3 that is at least 100 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:3 that is at least 25 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:4 that is at least 105 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:4 that is at least 25 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:5 that is at least 90 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:5 that is at least 20 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:6 that is at least 110 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:6 that is at least 20 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:7 that is at least 100 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:7 that is at least 20 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:8 that is at least 100 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:8 that is at least 20 amino acid residues in length, or said amino acid sequence that is at least 85 percent identical to said C-terminal portion.

The invention will be further described in the following examples, which do not limit the scope of the invention described in the claims.

### EXAMPLES

### Example 1

### Materials and Methods

Bioinformatics analysis: Several DddAₜₒₓ-related proteins were identified using the core facilitating (e.g., “meltase”)/deaminase dual domain of the Burkholderia cenocepacia DddAₜₒₓ protein and its known domain mapping of the intrinsically encoded cytosine deaminase as a search query. Each likely cytosine deaminase was annotated, and the amino acid sequences of the remaining protein subdomains were aligned as potential facilitating domains (sometimes referred to as “modular meltases”). A phylogenetic tree was generated using these alignment parameters, and a set of candidate facilitating polypeptides was selected according to a range of evolutionary divergence for downstream studies. Seven candidates derived from different branches of this tree were then selected (TABLE 1) and evaluated to functionally determine their facilitating and intrinsic base editing efficiencies. The sequence alignment of those candidates was performed using CLUSTAL Omega v. 1.2.4 (default parameters) and visualized using Jalview software. The AI-based AlphaFold server was used to generate the protein models for those candidates. Alignment and structure-based studies were used to decide the split point in all candidates to avoid potential toxicity. The alignment data also were useful to predict the active site residue for generating dead versions to test the potential facilitating function when in conjunction with an extrinsic deaminase activity.

TABLE 1
Sequences tested

| | SEQ ID NO: | SEQ ID NO: | SEQ ID NOS: |
|---|---|---|---|
| Origin | (wt) | (inactivated by E→A) | (split sequences) |
| B. cenocepacia | 1 | 25 | 35 and 36 |
| B. gladioli | 2 | 26 | 37 and 38 |
| Caballeronia sp. | 3 | 27 | 39 and 40 |
| R. intestinalis | 4 | 28 | 41 and 42 |
| Falcatimonas sp. | 5 | 29 | 43 and 44 |
| Clostridium sp. | 6 | 30 | 45 and 46 |
| R. bicirculans | 7 | 31 | 47 and 48 |
| B. ubonensis | 8 | 32 | 49 and 50 |

Gene synthesis and plasmid construction: De novo gene synthesis was used for both the active and catalytic mutant forms of the seven facilitating domain candidates. The genes were codon optimized for expression in human cells. For testing the facilitating domain-enabled intrinsic C-to-T deaminase activity at the MT-ND4 locus, 14 different plasmids were prepared. The N- and C-terminal halves of each candidate polypeptide were amplified from the ordered gene blocks. PCR products were digested with Xbal and BamHI. Digested N- and C-terminal fragments were then cloned into the ND4-Right-DddAₜₒₓ-N and ND4-Left-DddAₜₒₓ-C vectors, respectively, which were linearized with Xbal and BamHI (Sabharwal et al., supra). To test the facilitating domain-enabled extrinsic A-to-G activity of the selected sequences, the predicted cytosine deaminase active site glutamate was mutated to alanine in each candidate to generate a putative mutant version for each (TABLE 1 and FIG. 2B). The E1347A DddAtex domain of the ND1 specific monomeric TALED (TALED_Left-ND1-AD-E1347A, addgene plasmid #183894) was replaced with the mutant versions of the polypeptides (SEQ ID NOS: 25-32; FIG. 2B). Full-length mutant proteins were amplified, and each amplicon was digested with Eag1 and Apal and then ligated into the TALED_Left-ND1-AD-E1347A vector linearized with Eag1 and Apal. The sequences of all plasmids were confirmed by whole plasmid sequencing (Primordium Labs; Arcadia, CA).

Cell culture and transfection: The HEK293T cell line was obtained from ATCC and cultured in DMEM media (Thermo Scientific) supplemented with 10% fetal bovine serum (Gibco) and 1×penicillin/streptomycin solution (Pen/Strep) (Thermo Scientific). The HEK293T cell line was maintained at 37° C. with 5% CO₂ and was passaged before cells reached 80% confluency. Prior to transfection, a total of 300,000 cells/well were seeded in six-well plate 18 hours before the time of lipofection with LIPOFECTAMINE″ 3000 (Thermo Scientific). When only one construct was required for the assay, the amount of transfected plasmid was 1000 ng; when two constructs were required, the total amount of plasmid was 1000 ng (500 ng each).

Genomic DNA isolation and mitochondrial genotyping: After 72 hours, media was aspirated, and cells were washed with 1× phosphate-buffered saline (Thermo Scientific). The transfected cells were harvested, and total genomic DNA was extracted from cells using the DNEASY® Blood and Tissue Kit (Qiagen). Primers flanking the target loci were used to amplify the edited loci using MyTaq polymerase (Bioline). Primer sequence details for gene cloning and genotyping are presented in TABLE 2. PCR amplicons were gel extracted and purified using the QIAQUICK® Gel Extraction Kit (Qiagen). Purified samples were submitted to Genewiz (GENEWIZ LLC) for Sanger sequencing. Mitochondrial heteroplasmy level was quantified using EditR software to predict the percentage of edits in the target loci.

TABLE 2
Primer sequences

| Primer name | Primer sequence | SEQ ID NO: |
|---|---|---|
| TadA8e-mTALED-FP | ACTGACTGCGGCCGCGTGGTGT | 54 |
| Seq1-mTALED-RP | ACTGACTGGGGCCCTTAACAACCGCCCTTCACAGGG | 55 |
| Seq2-mTALED-RP | ACTGACTGGGGCCCTTAGCACGCACCTCCTGAGGAT | 56 |
| Seq3-mTALED-RP | ACTGACTGGGGCCCTTAGTTGCCCTTATATCTGGGGCTGATC | 57 |
| Seq4-mTALED-RP | ACTGACTGGGGCCCTTATTTAGGCTTTTTAGGGATCTTGTCGTTC | 58 |
| Seq5-mTALED-RP | ACTGACTGGGGCCCTTACTTGTTAGGCTTAGGCACCTTGTT | 59 |
| Seq6-mTALED-RP | ACTGACTGGGGCCCTTACTTTATCTTTGGCACCGTTGAATT | 60 |
| Seq7-mTALED-RP | ACTGACTGGGGCCCTTATTTCTTGCTAGGGGCCTTGGG | 61 |
| Seq1-N-CD-FP | CGTGGTCTAGAGGATCTTACACACTGGGCTC | 62 |
| Seq1-N-CD-RP | GCCTCGGATCCGCCAGAACAACCG | 63 |
| Seq1-C-CD-FP | CGTGGTCTAGAAGCATCCCCGTGAAAAGAGGCG | 64 |
| Seq1-C-CD-RP | GCCTCGGATCCGCCAGAACAACCG | 65 |
| Seq2-N-CD-FP | CGTGGTCTAGAACTGAGACCTTCCCGCTCG | 66 |
| Seq2-N-CD-RP | GCCTCGGATCCGCCAGAACCTTCTGGAGGCACTACCG | 67 |
| Seq2-C-CD-FP | CGTGGTCTAGAGCAGTGCCCGTTAAGCG | 68 |
| Seq2-C-CD-RP | GCCTCGGATCCGCCAGAGCACGCACCTCCTGAGGA | 69 |
| Seq3-N-CD-FP | CGTGGTCTAGAAGCCAGTACCCCTGCAAG | 70 |
| Seq3-N-CD-RP | GCCTCGGATCCGCCAGAGTTCTCAGGAGGCACCACG | 71 |
| Seq3-C-CD-FP | CGTGGTCTAGAGCCGTGGCCAACAACAG | 72 |
| Seq3~C-CD-RP | GCCTCGGATCCGCCAGAGTTGCC | 73 |
| Seq4-N-CD-FP | CGTGGTCTAGAGGCGGTTCTTCTAGTATCAACCTGC | 74 |
| Seq4-N-CD-RP | GCCTCGGATCCGCCAGAATTCGCTGGTGGCACTACGG | 75 |
| Seq4-C-CD-FP | CGTGGTCTAGAGCAAACGCACCAAAACCCAG | 76 |
| Seq4-C-CD-RP | GCCTCGGATCCGCCAGATTTAGGCTTTTTAGGGATCTTGTCGTTC | 77 |
| Seq5-N-CD-FP | CGTGGTCTAGACAGAGCAACATGAGCATCAC | 78 |
| Seq5-N-CD-RP | GGCTCGGATCCGCCAGAGTTGATAGGTGGGATCACCTCCAG | 79 |
| Seq5-C-CD-FP | CGTGGTCTAGAGCCAAGGCCCCTAAACCC | 80 |
| Seq5-C-CD-RP | GCCTCGGATCCGCCAGACTTGTTAGGC | 81 |
| Seq6-N-CD-FP | CGTGGTCTAGAAATGCTAATTGCAATCAAGAGAAGCCC | 82 |
| Seq6-N-CD-RP | GCCTCGGATCCGCCAGAGTTACTGGGTGGAACGACGG | 83 |
| Seq6-C-CD-FP | CGTGGTCTAGATCAGTTGCCAACAATGTAAGAGCG | 84 |
| Seq6-C-CD-RP | GCCTCGGATCCGCCAGACTTTATCTTTGGCACCGTTGAATTAC | 85 |
| Seq7-N-CD-FP | CGTGGTCTAGAGCTCTGCTGAGAGAGG | 86 |
| Seq7-N-CD-RP | GCCTCGGATCCGCCAGAGTTCTCGGGGGGGATCAC | 87 |
| Seq7-C-CD-FP | CGTGGTCTAGAGCCAAGGCCCCAACAAG | 88 |
| Seq7-C-CD-RP | GCCTCGGATCCGCCAGATTTCTTGCTAG | 89 |
| ND1-genotyping-FP | CCCCAACGTTGTAGGCC | 90 |
| ND1-genotyping-RP | GGTTGGTCTCTGCTAGTGTGG | 91 |
| ND4-genotyping-FP | CACCGGCGCAGTCATTCTC | 92 |
| ND4-genotyping-RP | CATGGGGGCATGAGTTAGCAG | 93 |
| COX1-genotyping-FP | TACCCTCCCTTAGCAGGG | 113 |
| COX1-genotyping-RP | CTCGTGTGTCTACGTCTATTCC | 114 |

### Results

Sequence analysis: The polypeptide sequences of all seven potential candidates were aligned with the minimal domain from Burkholderia cenocepacia, and the putative cytosine deaminase active site residues were identified (FIG. 1). The split position was also determined by the alignment output and from predicted protein structure work. The potential catalytic glutamate residue of each sequence (boxed in FIG. 1) was mutated to alanine to generate the mutant versions used in the mTALED constructs.

Comparative extrinsic A-to-G editing: In the initial mTALED construct, the cytosine deaminase protein domain derived from B. cenocepacia was largely blocked using as little as a single amino acid substitution. This ‘dead’ polypeptide was shown to still encode a full dsDNA-enabling activity when fused to a separate adenosine deaminase, enabling A-to-G editing in the presumptive DNA ‘bubble’ generated by this polypeptide (Cho et al., supra). The catalytic mutant DddAₜₒₓ domain in the construct was replaced with the seven potential candidates described above (SEQ ID NOs: 26-32; FIG. 2B), and the resulting chimeric polypeptides (illustrated in FIG. 2A) were tested for the extrinsic A-to-G editing by AD. A range of meltase activity in these different clones, from no activity above background (SEQ ID NOs: 30 and 32), intermediate activity (SEQ ID NOs: 26, 29, and 31), and as high or higher activity than the reference clone (SEQ ID NOs: 27 and 28) (TABLE 3 and FIG. 2C). The target protospacer included three adenosine residues at positions 7, 10, and 12 of the opposite strand, where the A-to-G base editing was observed (T-to-C editing in the forward strand). The range of editing efficiency of AD at T10 and T12 was markedly higher when the meltase property of SEQ ID NO: 28 was used.

Comparative intrinsic C-to-T editing: To access and compare the meltase enabled intrinsic C-to-T editing of each of the selected polypeptide candidates, the cytosine on the reverse strand at the m.G11922 position of the MT-ND4 gene was selected (FIG. 3A). HEK293T cells were transfected with each of the monomers of the ND4-Left-(SEQ ID NOs: 35, 37, 39, 41, 43, 45, 47, and 49)-C and complementary ND4-Right-(SEQ ID NOs: 36, 38, 40, 42, 44, 46, 48, and 50)-N(left) (FIG. 3B), and the editing efficiencies were measured 72 hours after transfection. The editing activities of the potential meltase candidates were compared with the original ND4 specific FusXTBE construct (Sabbarwal et al., supra) targeting the same mtDNA locus. A range of meltase activity was observed with the different clones, from no activity significantly above background (SEQ ID NOs: 45/46 and 49/50), just above background (SEQ ID NO:43/44), intermediate activity (SEQ ID NOs: 41/42 and 47/48) and as high or higher activity than the reference clone (SEQ ID NOs: 37/38 and 39/40) (TABLE 4 and FIG. 3C). Intrinsic C-to-T editing was observed at the C5 and C8 positions of the protospacer, with a higher degree of editing occurring at C5 (the primary target).

TABLE 3
Extrinsic A to G editing

Extrinsic activity

| Origin | T₇ | T₁₀ | T₁₂ |
|---|---|---|---|
| B. cenocepacia | 17 ± 0.57 | 8 ± 1 | 6 ± 0.57 |
| B. gladioli | 13 ± 2 | 6 ± 5.5 | 2 ± 0.57 |
| Caballeronia sp. | 19 ± 2 | 12 ± 1.5 | 9 ± 1.15 |
| R. intestinalis | 18 ± 0.57 | 20 ± 0.57 | 20 ± 1 |
| Falcatimonas sp. | 15 | 8 ± 0.57 | 6 ± 0.57 |
| Clostridium sp. | 6 ± 1.15 | 3 ± 0.57 | 4 ± 1.15 |
| R. bicirculans | 11 ± 1 | 7 ± 0 | 4 ± 0 |
| B. ubonensis | 5 ± 0 | 3 ± 0.57 | 3 ± 0 |

TABLE 4
Intrinsic C to T editing for split facilitating domains

Intrinsic activity

| Origin | C₅ | C₈ |
|---|---|---|
| B. cenocepacia | 27.7 ± 1.4 | 7 ± 0.92 |
| B. gladioli | 30 ± 1.6 | 9 ± 0 |
| Caballeronia sp. | 29.7 ± 0.53 | 1.3 ± 0.53 |
| R. intestinalis | 23.3 ± 0.53 | 3.7 ± 2.13 |
| Falcatimonas sp. | 8.7 ± 0.7 | 2 ± 0 |
| Clostridium sp. | 5.6 ± 0.53 | 2 ± 0 |
| R. bicirculans | 25.7 ± 0.53 | 1 ± 0 |
| B. ubonensis | 7.3 ± 2.7 | 1.7 ± 0.53 |

### Example 2

The results in this Example re-present and expand on at least some of the results provided in other Examples.

### Materials and Methods

Bioinformatics analysis and selection of candidate DddAₜₒₓ homologs: To identify DddAₜₒₓ-related proteins, a bioinformatics approach using the core meltase/deaminase dual domain from B. cenocepacia and its known domain mapping of the intrinsically encoded cytosine deaminase was employed as a search query in a current public database. Each putative cytosine deaminase was then annotated and the remaining protein subdomains were aligned to identify potential dsDNA modular meltases. A phylogenetic tree was generated based on these alignment parameters to select a set of candidate meltase proteins, considering a range of evolutionary divergence for subsequent investigations. From this tree, seven candidates representing different branches were selected for functional analysis of their meltase and intrinsic base editing efficiencies (TABLE 5). Sequence alignment of these candidates was performed using CLUSTAL Omega v.1.2.4 with default parameters, and the results were visualized using Jalview software. For structural insights, the AI-based AlphaFold server was used to generate protein models for the selected candidates. Additionally, the alignment data assisted with identification of the optimal split point in all candidates to avoid potential toxicity. The alignment information also used to predict the active site residues, enabling the generation of “dead” versions of the candidates for testing potential meltase activity in conjunction with an extrinsic deaminase activity.

TABLE 5
Short-listed sequences for activity testing and their origin

| SEQ ID NO: | Origin | NCBI Accession | Rename |
|---|---|---|---|
| 1 | B. cenocepacia | WP_212255806.1 | Mlt_Bc |
| 2 | B. gladioli | WP_186127404.1 | Mlt_Bg |
| 3 | Caballeronia sp | WP_250441259.1 | Mlt_Cab |
| 4 | R. intestinalis | WP_195441564.1 | Mlt_Ri |
| 5 | Falcatimonas sp. | WP_216577045.1 | Mlt_Fal |
| 6 | Clostridium sp. | NLI59004.1 | Mlt_Clo |
| 7 | R. bicirculans | MBS4926674.1 | Mlt_Rb |
| 8 | B. ubonensis | WP_059728184 | Mlt_Bu |

Gene synthesis and plasmid construction: De novo gene synthesis was performed for all seven meltase candidates, generating both active and catalytic mutant forms. In each candidate, the predicted cytosine deaminase active site glutamate (boxed in FIG. 1) was modified to alanine, resulting in putative mutant versions. These genes were codon optimized for expression in human cells.

Plasmid construction for intrinsic C-to-T activity: To test the meltase-enabled intrinsic C-to-T deaminase activity at the MT-ND4 locus, 14 plasmids were prepared. The N- and C-terminal halves of each candidate were amplified from ordered gene blocks and digested with the restriction enzymes Xbal and BamHI. The digested fragments were cloned into the ND4-Right-DddAₜₒₓ-1397N and ND4-Left-DddAₜₒₓ-1397C vectors, respectively (Sabharwal et al., supra), which were linearized using Xbal and BamHI.

Plasmid construction for meltase-assisted extrinsic A-to-G activity: To test the meltase-enabled extrinsic A-to-G activity of the selected sequences, the E1347A DddAₜₒₓ domain of the ND1-specific monomeric A-to-G editors called TALEDs (TALED_Left-ND1-AD-E1347A, addgene plasmid #183894; Cho et al., supra) was replaced. Additionally, the extrinsic A-to-G meltase activity of sequence 4 (Mlt_Ri) was investigated in both dimeric and split designs. In the dimeric design, the E1347A DddAₜₒₓ domain of the ND1-specific dimeric left TALED (TALED_Left-ND1-E1347A, addgene plasmid #183895) was replaced with a mutant version (E2328A) of Mlt_Ri. This mutant plasmid was co-transfected with the ND1-specific dimeric right TALED (TALED_Right-ND1-AD, addgene plasmid #183900). For the split design, the N and C termini of Mlt_Bc (DddAₜₒₓ) were replaced with the N (2265-2378 aa) and C (2379-2409 aa) termini of Mlt_Ri in the ND1-specific split right TALED (TALED_Right-ND1-1397N, addgene plasmid #183898) and split left TALED (TALED_Left-ND1-1397C-AD, addgene plasmid #183892), respectively. All plasmids were confirmed through whole plasmid sequencing (Primordium Labs).

The extrinsic A-to-G editing acting also was tested via RNA delivery in monomeric TALED format for different target sites. Two destination plasmids were constructed with sequences encoding Mlt_Bc and Mlt_Ri as meltases and having a T3 promoter for RNA production. These vectors were compatible for the FusXTBE assembly protocol to make them destined for target sites (Sabharwal et al., supra; and Kar et al., supra). After the assembly process, the final plasmids were sequence verified by whole plasmid sequencing (Primordium Labs). Sequence-verified plasmids were linearized with PstI. The resulting linearized plasmids were employed as templates for in vitro mRNA synthesis using the T3 mMESSAGE mMACHINE™ Transcription Kit (Thermo Scientific, AM1348). The products of in vitro transcription subsequently underwent a polyadenylation step to yield complete synthetic mRNA using the Poly (A) Tailing Kit from Thermo Scientific (AM1350). Following the poly(A) sitailing, the mRNA was purified using the MONARCH® RNA Cleanup Kit (New England Biolabs, #T2040). In the context of mitoBE RNA fibroblast electroporations, EP buffer (Etta Biotech Co.) was added to 9 μg of each RNA, resulting in a final volume of 15 μL. The diluted RNA solution was then mixed with 300 μL of fibroblast cells at a concentration of 20×10⁶ cells/mL in EP buffer. Next, 105 μL of this cell and RNA mixture was added into separate cuvettes in triplicates. Fibroblast cells were electroporated with an Etta H1 electroporator (Etta Biotech Co.) using the following parameters: 200 V, 784 ms interval, six pulses, and 1,000 μs duration. Cells from each cuvette were then transferred to a single well of a six-well tissue cell culture plate containing 3 mL of complete DMEM.

Plasmid construction for meltase-assisted extrinsic C-to-T activity: To investigate the meltase-assisted extrinsic C-to-T deaminase activity, the ND4 locus on human mitochondrial DNA was targeted. The catalytically dead form of Mlt_Bc and Mlt_Ri was amplified from ordered gene blocks and cloned to generate the Cox1-Left-Mlt_Bc-UGI and Cox1-Left-Mlt_Ri-UGI plasmids, respectively. These plasmids were designed to incorporate the meltase enzymes Mlt_Bc and Mlt_Ri, along with the UGI enzyme known for inhibiting uracil excision activity. The ND4-Left-Mlt_Bc-UGI and ND4-Left-Mlt_Ri-UGI plasmids were independently co-transfected with specific right TALE constructs containing active cytosine deaminases (APOBEC1, AID, TadCBEd, or CBET), targeting the ND4 locus. All plasmids were confirmed through whole plasmid sequencing (Primordium Labs).

Cell Culture and Transfection: The HEK293T cell line was obtained from ATCC and cultured in DMEM media (Thermo Scientific) supplemented with 10% fetal bovine serum (Gibco) and 1× penicillin/streptomycin solution (Pen/Strep) (Thermo Scientific). The cells were maintained at 37° C. with 5% CO₂ and passaged when they reached 80% confluency. For transfection, a total of 300,000 cells/well were seeded in a six-well plate 18 hours before lipofection using LIPOFECTAMINE™ 3000 (Thermo Scientific). When conducting an assay with a single construct, the total amount of transfected plasmid was 1000 ng. For assays requiring two constructs, the total amount of plasmid was 1000 ng, with 500 ng for each construct.

Genomic DNA isolation and mitochondrial genotyping: After 72 hours, the media was aspirated and cells were washed with 1× phosphate-buffered saline (Thermo Scientific). Transfected cells were harvested, and total genomic DNA was extracted using the DNEASY® Blood and Tissue Kit (Qiagen). For genotyping, primers flanking the target loci were used to amplify the edited regions with MyTaq polymerase (Bioline). The primer sequences for gene cloning and genotyping are presented in TABLE 1 above. The PCR amplicons were purified using the QIAQUICK® Gel Extraction Kit (Qiagen) after gel extraction. The purified samples were then sent to Genewiz (GENEWIZ LLC) for Sanger sequencing to confirm the edits in the target loci. Mitochondrial heteroplasmy levels were quantified using EditR software, enabling prediction of the percentage of edits at the target loci.

### Results

Sequence analysis: The protein sequences of all seven potential candidates were aligned with the minimal domain from B. cenocepacia, and putative cytosine deaminase active site residues were identified (FIG. 1). The split positions were determined both from the alignment output and predicted protein structure analysis. To generate the mutant versions used in the TALED constructs, the potential catalytic glutamate residue of each sequence (boxed in FIG. 1) was mutated to alanine.

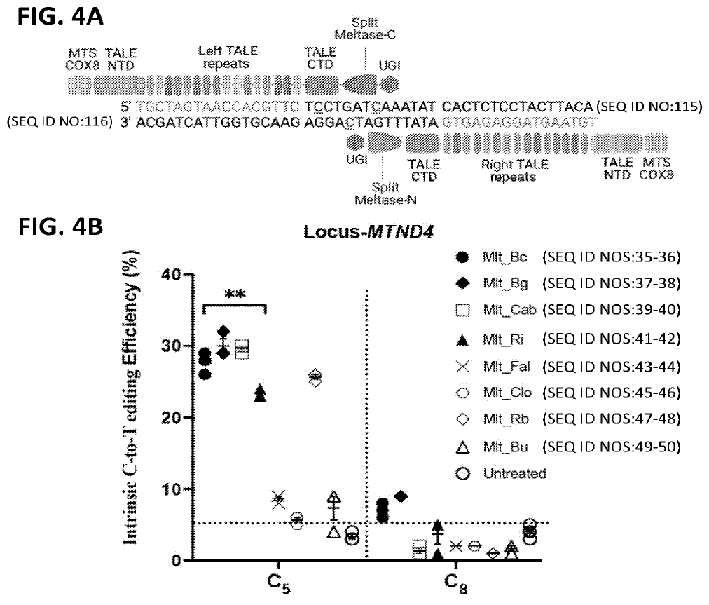

Comparative intrinsic C-to-T editing: Studies to assess and compare the meltase-enabled intrinsic C-to-T editing of each selected protein candidate were focused on the cytosine at the m.G11922 position of the MT-ND4 gene (FIG. 4A). HEK293T cells were transfected with each monomer of the ND4-LeftTALE [1-8]-C and the complementary ND4-Right-TALE [1-8]-N(left). Editing efficiencies were measured 72 hours after transfection. The editing activities of the potential meltase candidates were compared with the original ND4-specific FusXTBE construct (Sabharwal et al., supra) targeted to the same mtDNA locus. These studies revealed a range of meltase activity across different clones, from no activity significantly above background (Mlt_Clo and Mlt_Bu), slightly above background (Mlt_Fal), intermediate activity (Mlt_Ri and Mlt_Rb), to activity equal to or higher than the reference clone (Mlt_Bg and Mlt_Cab) (FIG. 4B).

Comparative extrinsic A-to-G editing: In the initial mTALED construct, the cytosine deaminase protein domain from B. cenocepacia was effectively blocked with as little as a single amino acid substitution. However, the resulting “dead” protein still retained its full dsDNA-enabling activity when fused to a separate adenosine deaminase (AD), enabling efficient A-to-G editing within the presumed DNA ‘bubble’ created by the meltase (Cho et al., supra). To explore the extrinsic A-to-G editing capabilities of the seven potential candidates, the mTALED design was modified to replace the catalytic mutant DddAₜₒₓ domain in the construct with each candidate, and the candidates were then tested for AD-assisted A-to-G editing (FIG. 5A). These studies revealed a diverse range of meltase activities among the different clones (FIG. 5B). Some clones exhibited no activity above background (Mlt_Clo and Mlt_Bu), while others displayed intermediate activity (Mlt_Bg, Mlt_Fal, and Mlt_Rb). Interestingly, certain clones demonstrated A-to-G editing activity that surpassed even the reference clone (Mlt_Cab and Mlt_Ri). In fact, the meltase property of Mlt_Ri showed significantly improved editing efficiency of AD at the T10 and T12 positions.

The aforementioned ND1-specific experiment was conducted using plasmid DNA delivery, and the results distinctly showcased that Mlt_Ri exhibited enhanced activity for extrinsic A-to-G activity in comparison to Mlt_Bc. A further investigation was undertaken to investigate the function of Mlt_Bc and Mlt_Ri within the framework of mTALED formats at MTND1, MTND5, MTCOX3, and MTTRNF target sites, this time employing RNA delivery using human primary fibroblast cells. At the ND1 locus, the extrinsic A-to-G base editing activity exhibited a notably higher average when the mTALED constructs were introduced via RNA electroporation, as opposed to plasmid DNA transfection (FIG. 6A vs. FIG. 5B). Moreover, a broader window of meltase-facilitated A-to-G editing activity was noted for both Mlt_Bc and Mlt_Ri at the ND1 target site (T15 position), when delivery was executed through RNA electroporation (FIG. 6A). This observation may be attributed to the presence of a slightly extended linker connecting the TALE repeats and the AD. Mlt_Ri consistently outperformed Mlt_Bc across multiple target sites in the context of meltase-assisted extrinsic A-to-G editing (FIGS. 6A-6D). These findings collectively indicated a heightened extrinsic A-to-G base editing activity on average when Mlt_Ri was employed as the meltase.

The mTALED experiments indicated that Mlt_Ri outperformed Mlt_Bc in meltase-assisted A-to-G editing (FIGS. 5B and 6B). Consequently, further studies were focused on Mlt_Ri, both in dimeric and split setups. In the dTALED design, the mutant version (E>A) of Mlt_Bc and Mlt_Ri proteins were tethered to the left arm of ND1-specific TALE, while the extrinsic base editing experiment was conducted alongside ND1-specific right TALE linked to AD (FIG. 7A). Remarkably, the overall extrinsic A-to-G activity was enhanced when Mlt_Ri was used as the meltase (FIG. 7B). Unexpected C-to-T editing also was observed at the C11 position.

The extrinsic A-to-G editing efficiency in the split version of TALED was further investigated (FIG. 8A). These studies demonstrated that the split of Mlt_Ri at position 2378N (corresponding to the 1397G split position of Mlt_Bc) displayed lower average extrinsic A-to-G activity compared to the split version of Mlt_Bc (FIG. 8B).

Comparative extrinsic C-to-T editing: To investigate the potential of the DNA meltases to enable extrinsic C-to-T editing, the dTALED setup was reconfigured and plasmids were designed for testing meltase-assisted extrinsic C-to-T editing. In particular, the left TALE (specific to the Cox1 locus) was linked either to mutant Mlt_Bc or Mlt_Ri and a UGI molecule (FIG. 9A). These constructs were then co-transfected with the Cox1-specific right TALE, tethered to a cytidine deaminase (APOBEC1/AID/TadCBEd/CBET) and a UGI molecule. The overall extrinsic C-to-T activity, or G-to-A editing on the opposite strand, assisted by Mlt_Ri at G8 position was about two-fold higher than the outcomes assisted by Mlt_Bc (FIG. 9B). Further, every cytidine deaminase (CD) assisted by Mlt_Ri exhibited higher activity compared to its corresponding Mlt_Bc-assisted counterpart (FIG. 9B). Moreover, the experiments with CBET showed the highest extrinsic C-to-T editing at the Cox1 locus when assisted by Mlt_Ri (FIG. 9B). This enhanced activity indicated that Mlt_Ri can serve as a highly efficient meltase in facilitating extrinsic C-to-T editing processes.

In further studies, the extrinsic A-to-G editing of chimeric polypeptides having an exemplary monomeric format mitochondrial base editor design depicted in FIG. 10A as tested. The facilitating domain in the polypeptides contained SEQ ID NOS: 1 and 9-13, where each of the amino acid sequences set forth in SEQ ID NOS: 1 and 9-13 was modified to reduce or abolish cytosine deaminase activity. Editing of a human mitochondrial NDI gene sequence (SEQ ID NOS: 123 and 124) was tested in 293T cells. Results are plotted in FIG. 10B, which shows that the Trepanoma sp.-derived sequence (SEQ ID NO:98) demonstrated the greatest level of editing efficiency.

TABLE 6
Sequences tested

| | SEQ ID NO: | SEQ ID NO: |
|---|---|---|
| Origin | (wt) | (inactivated by E→A) |
| B. cenocepacia | 1 | 25 |
| Treponema sp. | 9 | 98 |
| P. guartelaensis | 10 | 99 |
| C. bacterium | 11 | 100 |
| P. violaceinigra | 12 | 101 |
| P. fastidiosa | 13 | 102 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A chimeric polypeptide comprising a nucleic acid interacting domain and a facilitating domain, wherein said facilitating domain comprises:
an amino acid sequence (i) that comprises the amino acid sequence set forth in SEQ ID NO: 2, (ii) that is at least 92 percent identical to the amino acid sequence set forth in SEQ ID NO: 2, (iii) that comprises an N-terminal portion of the amino acid sequence set forth in SEQ ID NO: 2 that is at least 100 amino acid residues in length, (iv) that is at least 92 percent identical to said N-terminal portion, (v) that comprises a C-terminal portion of the amino acid sequence set forth in SEQ ID NO:2 that is at least 25 amino acid residues in length, or (vi) that is at least 92 percent identical to said C-terminal portion;
an amino acid sequence (i) that comprises the amino acid sequence set forth in SEQ ID NO: 3, (ii) that is at least 85 percent identical to the amino acid sequence set forth in SEQ ID NO: 3, (iii) that comprises an N-terminal portion of the amino acid sequence set forth in SEQ ID NO: 3 that is at least 100 amino acid residues in length, (iv) that is at least 85 percent identical to said N-terminal portion, (v) that comprises a C-terminal portion of the amino acid sequence set forth in SEQ ID NO:3 that is at least 25 amino acid residues in length, or (vi) that is at least 85 percent identical to said C-terminal portion;
an amino acid sequence (i) that comprises the amino acid sequence set forth in SEQ ID NO: 4, (ii) that is at least 85 percent identical to the amino acid sequence set forth in SEQ ID NO: 4, (iii) that comprises an N-terminal portion of the amino acid sequence set forth in SEQ ID NO: 4 that is at least 105 amino acid residues in length, (iv) that is at least 85 percent identical to said N-terminal portion, (v) that comprises a C-terminal portion of the amino acid sequence set forth in SEQ ID NO:4 that is at least 25 amino acid residues in length, or (vi) that is at least 85 percent identical to said C-terminal portion;
an amino acid sequence (i) that comprises the amino acid sequence set forth in SEQ ID NO: 5, (ii) that is at least 85 percent identical to the amino acid sequence set forth in SEQ ID NO: 5, (iii) that comprises an N-terminal portion of the amino acid sequence set forth in SEQ ID NO: 5 that is at least 90 amino acid residues in length, (iv) that is at least 85 percent identical to said N-terminal portion, (v) that comprises a C-terminal portion of the amino acid sequence set forth in SEQ ID NO:5 that is at least 20 amino acid residues in length, or (vi) that is at least 85 percent identical to said C-terminal portion;
an amino acid sequence (i) that comprises the amino acid sequence set forth in SEQ ID NO: 6, (ii) that is at least 85 percent identical to the amino acid sequence set forth in SEQ ID NO: 6, (iii) that comprises an N-terminal portion of the amino acid sequence set forth in SEQ ID NO: 6 that is at least 110 amino acid residues in length, (iv) that is at least 85 percent identical to said N-terminal portion, (v) that comprises a C-terminal portion of the amino acid sequence set forth in SEQ ID NO:6 that is at least 20 amino acid residues in length, or (vi) that is at least 85 percent identical to said C-terminal portion;
an amino acid sequence (i) that comprises the amino acid sequence set forth in SEQ ID NO: 7, (ii) that is at least 85 percent identical to the amino acid sequence set forth in SEQ ID NO: 7, (iii) that comprises an N-terminal portion of the amino acid sequence set forth in SEQ ID NO: 7 that is at least 100 amino acid residues in length, (iv) that is at least 85 percent identical to said N-terminal portion, (v) that comprises a C-terminal portion of the amino acid sequence set forth in SEQ ID NO:7 that is at least 20 amino acid residues in length, or (vi) that is at least 85 percent identical to said C-terminal portion;
an amino acid sequence (i) that comprises the amino acid sequence set forth in SEQ ID NO: 8, (ii) that is at least 85 percent identical to the amino acid sequence set forth in SEQ ID NO: 8, (iii) that comprises an N-terminal portion of the amino acid sequence set forth in SEQ ID NO: 8 that is at least 100 amino acid residues in length, (iv) that is at least 85 percent identical to said N-terminal portion, (v) that comprises a C-terminal portion of the amino acid sequence set forth in SEQ ID NO:8 that is at least 20 amino acid residues in length, or (vi) that is at least 85 percent identical to said C-terminal portion;
an amino acid sequence (i) that comprises the amino acid sequence set forth in SEQ ID NO: 9, (ii) that is at least 90 percent identical to the amino acid sequence set forth in SEQ ID NO: 9, (iii) that comprises an N-terminal portion of the amino acid sequence set forth in SEQ ID NO: 9 that is at least 100 amino acid residues in length, (iv) that is at least 93% identical to said N-terminal portion, (v) that comprises a C-terminal portion of the amino acid sequence set forth in SEQ ID NO:9 that is at least 23 amino acid residues in length, or (vi) that is at least 88% identical to said C-terminal portion;
an amino acid sequence (i) that comprises the amino acid sequence set forth in SEQ ID NO: 10, (ii) that is at least 90 percent identical to the amino acid sequence set forth in SEQ ID NO: 10, (iii) that comprises an N-terminal portion of the amino acid sequence set forth in SEQ ID NO: 10 that is at least 105 amino acid residues in length, (iv) that is at least 94% identical to said N-terminal portion, (v) that comprises a C-terminal portion of the amino acid sequence set forth in SEQ ID NO:10 that is at least 23 amino acid residues in length, or (vi) that is at least 88% identical to said C-terminal portion;
an amino acid sequence (i) that comprises the amino acid sequence set forth in SEQ ID NO: 11, (ii) that is at least 90 percent identical to the amino acid sequence set forth in SEQ ID NO: 11, (iii) that comprises an N-terminal portion of the amino acid sequence set forth in SEQ ID NO: 11 that is at least 100 amino acid residues in length, (iv) that is at least 93% identical to said N-terminal portion, (v) that comprises a C-terminal portion of the amino acid sequence set forth in SEQ ID NO:11 that is at least 25 amino acid residues in length, or (vi) that is at least 86% identical to said C-terminal portion;
an amino acid sequence (i) that comprises the amino acid sequence set forth in SEQ ID NO: 12, (ii) that is at least 90 percent identical to the amino acid sequence set forth in SEQ ID NO: 12, (iii) that comprises an N-terminal portion of the amino acid sequence set forth in SEQ ID NO: 12 that is at least 108 amino acid residues in length, (iv) that is at least 93% identical to said N-terminal portion, (v) that comprises a C-terminal portion of the amino acid sequence set forth in SEQ ID NO: 12 that is at least 23 amino acid residues in length, or (vi) that is at least 85% identical to said C-terminal portion; or
an amino acid sequence (i) that comprises the amino acid sequence set forth in SEQ ID NO: 13, (ii) that is at least 90 percent identical to the amino acid sequence set forth in SEQ ID NO: 13, (iii) that comprises an N-terminal portion of the amino acid sequence set forth in SEQ ID NO: 13 that is at least 116 amino acid residues in length, (iv) that is at least 93% identical to said N-terminal portion, (v) that comprises a C-terminal portion of the amino acid sequence set forth in SEQ ID NO:13 that is at least 18 amino acid residues in length, or (vi) that is at least 85% identical to said C-terminal portion.

2. The chimeric polypeptide of claim 1, wherein said nucleic acid interacting domain is N-terminal to said facilitating domain.

3-5. (canceled)

6. The chimeric polypeptide of claim 1, further comprising a linker between said nucleic acid interacting domain and said facilitating domain.

7. The chimeric polypeptide of claim 1, further comprising a mitochondrial targeting sequence (MTS).

8. The chimeric polypeptide of claim 7, wherein said MTS is an isocitrate dehydrogenase 2 MTS, a human COX8A MTS, or a human SOD2 MTS.

9. The chimeric polypeptide of claim 7, wherein said MTS is at the N-terminus of said chimeric polypeptide.

10. The chimeric polypeptide of claim 9, wherein said chimeric polypeptide comprises, in order from N-terminus to C-terminus, said MTS, said nucleic acid interacting domain, and said facilitating domain, and wherein said chimeric polypeptide further comprises a linker between said MTS and said nucleic acid interacting domain.

11-19. (canceled)

20. A nucleic acid comprising a nucleotide sequence encoding a chimeric polypeptide comprising a nucleic acid interacting domain and a facilitating domain, wherein said facilitating domain comprises:
an amino acid sequence (i) that comprises the amino acid sequence set forth in SEQ ID NO: 2, (ii) that is at least 92 percent identical to the amino acid sequence set forth in SEQ ID NO: 2, (iii) that comprises an N-terminal portion of the amino acid sequence set forth in SEQ ID NO: 2 that is at least 100 amino acid residues in length, (iv) that is at least 92 percent identical to said N-terminal portion, (v) that comprises a C-terminal portion of the amino acid sequence set forth in SEQ ID NO:2 that is at least 25 amino acid residues in length, or (vi) that is at least 92 percent identical to said C-terminal portion;
an amino acid sequence (i) that comprises the amino acid sequence set forth in SEQ ID NO: 3, (ii) that is at least 85 percent identical to the amino acid sequence set forth in SEQ ID NO: 3, (iii) that comprises an N-terminal portion of the amino acid sequence set forth in SEQ ID NO: 3 that is at least 100 amino acid residues in length, (iv) that is at least 85 percent identical to said N-terminal portion, (v) that comprises a C-terminal portion of the amino acid sequence set forth in SEQ ID NO:3 that is at least 25 amino acid residues in length, or (vi) that is at least 85 percent identical to said C-terminal portion;
an amino acid sequence (i) that comprises the amino acid sequence set forth in SEQ ID NO: 4, (ii) that is at least 85 percent identical to the amino acid sequence set forth in SEQ ID NO: 4, (iii) that comprises an N-terminal portion of the amino acid sequence set forth in SEQ ID NO: 4 that is at least 105 amino acid residues in length, (iv) that is at least 85 percent identical to said N-terminal portion, (v) that comprises a C-terminal portion of the amino acid sequence set forth in SEQ ID NO:4 that is at least 25 amino acid residues in length, or (vi) that is at least 85 percent identical to said C-terminal portion;
an amino acid sequence (i) that comprises the amino acid sequence set forth in SEQ ID NO: 5, (ii) that is at least 85 percent identical to the amino acid sequence set forth in SEQ ID NO: 5, (iii) that comprises an N-terminal portion of the amino acid sequence set forth in SEQ ID NO: 5 that is at least 90 amino acid residues in length, (iv) that is at least 85 percent identical to said N-terminal portion, (v) that comprises a C-terminal portion of the amino acid sequence set forth in SEQ ID NO:5 that is at least 20 amino acid residues in length, or (vi) that is at least 85 percent identical to said C-terminal portion;
an amino acid sequence (i) that comprises the amino acid sequence set forth in SEQ ID NO: 6, (ii) that is at least 85 percent identical to the amino acid sequence set forth in SEQ ID NO: 6, (iii) that comprises an N-terminal portion of the amino acid sequence set forth in SEQ ID NO: 6 that is at least 110 amino acid residues in length, (iv) that is at least 85 percent identical to said N-terminal portion, (v) that comprises a C-terminal portion of the amino acid sequence set forth in SEQ ID NO:6 that is at least 20 amino acid residues in length, or (vi) that is at least 85 percent identical to said C-terminal portion;
an amino acid sequence (i) that comprises the amino acid sequence set forth in SEQ ID NO: 7, (ii) that is at least 85 percent identical to the amino acid sequence set forth in SEQ ID NO: 7, (iii) that comprises an N-terminal portion of the amino acid sequence set forth in SEQ ID NO: 7 that is at least 100 amino acid residues in length, (iv) that is at least 85 percent identical to said N-terminal portion, (v) that comprises a C-terminal portion of the amino acid sequence set forth in SEQ ID NO:7 that is at least 20 amino acid residues in length, or (vi) that is at least 85 percent identical to said C-terminal portion;
an amino acid sequence (i) that comprises the amino acid sequence set forth in SEQ ID NO: 8, (ii) that is at least 85 percent identical to the amino acid sequence set forth in SEQ ID NO: 8, (iii) that comprises an N-terminal portion of the amino acid sequence set forth in SEQ ID NO: 8 that is at least 100 amino acid residues in length, (iv) that is at least 85 percent identical to said N-terminal portion, (v) that comprises a C-terminal portion of the amino acid sequence set forth in SEQ ID NO:8 that is at least 20 amino acid residues in length, or (vi) that is at least 85 percent identical to said C-terminal portion;
an amino acid sequence (i) that comprises the amino acid sequence set forth in SEQ ID NO: 9, (ii) that is at least 90 percent identical to the amino acid sequence set forth in SEQ ID NO: 9, (iii) that comprises an N-terminal portion of the amino acid sequence set forth in SEQ ID NO: 9 that is at least 100 amino acid residues in length, (iv) that is at least 93% identical to said N-terminal portion, (v) that comprises a C-terminal portion of the amino acid sequence set forth in SEQ ID NO:9 that is at least 23 amino acid residues in length, or (vi) that is at least 88% identical to said C-terminal portion;
an amino acid sequence (i) that comprises the amino acid sequence set forth in SEQ ID NO: 10, (ii) that is at least 90 percent identical to the amino acid sequence set forth in SEQ ID NO: 10, (iii) that comprises an N-terminal portion of the amino acid sequence set forth in SEQ ID NO: 10 that is at least 105 amino acid residues in length, (iv) that is at least 94% identical to said N-terminal portion, (v) that comprises a C-terminal portion of the amino acid sequence set forth in SEQ ID NO:10 that is at least 23 amino acid residues in length, or (vi) that is at least 88% identical to said C-terminal portion;
an amino acid sequence (i) that comprises the amino acid sequence set forth in SEQ ID NO: 11, (ii) that is at least 90 percent identical to the amino acid sequence set forth in SEQ ID NO: 11, (iii) that comprises an N-terminal portion of the amino acid sequence set forth in SEQ ID NO: 11 that is at least 100 amino acid residues in length, (iv) that is at least 93% identical to said N-terminal portion, (v) that comprises a C-terminal portion of the amino acid sequence set forth in SEQ ID NO:11 that is at least 25 amino acid residues in length, or (vi) that is at least 86% identical to said C-terminal portion;
an amino acid sequence (i) that comprises the amino acid sequence set forth in SEQ ID NO: 12, (ii) that is at least 90 percent identical to the amino acid sequence set forth in SEQ ID NO: 12, (iii) that comprises an N-terminal portion of the amino acid sequence set forth in SEQ ID NO: 12 that is at least 108 amino acid residues in length, (iv) that is at least 93% identical to said N-terminal portion, (v) that comprises a C-terminal portion of the amino acid sequence set forth in SEQ ID NO:12 that is at least 23 amino acid residues in length, or (vi) that is at least 85% identical to said C-terminal portion; or
an amino acid sequence (i) that comprises the amino acid sequence set forth in SEQ ID NO: 13, (ii) that is at least 90 percent identical to the amino acid sequence set forth in SEQ ID NO: 13, (iii) that comprises an N-terminal portion of the amino acid sequence set forth in SEQ ID NO: 13 that is at least 116 amino acid residues in length, (iv) that is at least 93% identical to said N-terminal portion, (v) that comprises a C-terminal portion of the amino acid sequence set forth in SEQ ID NO:13 that is at least 18 amino acid residues in length, or (vi) that is at least 85% identical to said C-terminal portion.

21-22. (canceled)

23. A method for generating a mutation within mitochondrial or nuclear genomic DNA of a cell, comprising:
(a) introducing, into the cell, a chimeric polypeptide comprising a nucleic acid interacting domain and a facilitating domain, wherein said facilitating domain comprises:
(1) an amino acid sequence (i) that comprises the amino acid sequence set forth in SEQ ID NO:2, (ii) that is at least 92 percent identical to the amino acid sequence set forth in SEQ ID NO:2, (iii) that comprises an N-terminal portion of the amino acid sequence set forth in SEQ ID NO:2 that is at least 100 amino acid residues in length, (iv) that is at least 92 percent identical to said N-terminal portion, (v) that comprises a C-terminal portion of the amino acid sequence set forth in SEQ ID NO:2 that is at least 25 amino acid residues in length, or (vi) that is at least 92 percent identical to said C-terminal portion;
(2) an amino acid sequence (i) that comprises the amino acid sequence set forth in SEQ ID NO:3, (ii) that is at least 85 percent identical to the amino acid sequence set forth in SEQ ID NO:3, (iii) that comprises an N-terminal portion of the amino acid sequence set forth in SEQ ID NO:3 that is at least 100 amino acid residues in length, (iv) that is at least 85 percent identical to said N-terminal portion, (v) that comprises a C-terminal portion of the amino acid sequence set forth in SEQ ID NO:3 that is at least 25 amino acid residues in length, or (vi) that is at least 85 percent identical to said C-terminal portion;
(3) an amino acid sequence (i) that comprises the amino acid sequence set forth in SEQ ID NO:4, (ii) that is at least 85 percent identical to the amino acid sequence set forth in SEQ ID NO:4, (iii) that comprises an N-terminal portion of the amino acid sequence set forth in SEQ ID NO:4 that is at least 105 amino acid residues in length, (iv) that is at least 85 percent identical to said N-terminal portion, (v) that comprises a C-terminal portion of the amino acid sequence set forth in SEQ ID NO:4 that is at least 25 amino acid residues in length, or (vi) that is at least 85 percent identical to said C-terminal portion;
(4) an amino acid sequence (i) that comprises the amino acid sequence set forth in SEQ ID NO:5, (ii) that is at least 85 percent identical to the amino acid sequence set forth in SEQ ID NO:5, (iii) that comprises an N-terminal portion of the amino acid sequence set forth in SEQ ID NO:5 that is at least 90 amino acid residues in length, (iv) that is at least 85 percent identical to said N-terminal portion, (v) that comprises a C-terminal portion of the amino acid sequence set forth in SEQ ID NO:5 that is at least 20 amino acid residues in length, or (vi) that is at least 85 percent identical to said C-terminal portion;
(5) an amino acid sequence (i) that comprises the amino acid sequence set forth in SEQ ID NO:6, (ii) that is at least 85 percent identical to the amino acid sequence set forth in SEQ ID NO:6, (iii) that comprises an N-terminal portion of the amino acid sequence set forth in SEQ ID NO:6 that is at least 110 amino acid residues in length, (iv) that is at least 85 percent identical to said N-terminal portion, (v) that comprises a C-terminal portion of the amino acid sequence set forth in SEQ ID NO:6 that is at least 20 amino acid residues in length, or (vi) that is at least 85 percent identical to said C-terminal portion;
(6) an amino acid sequence (i) that comprises the amino acid sequence set forth in SEQ ID NO:7, (ii) that is at least 85 percent identical to the amino acid sequence set forth in SEQ ID NO:7, (iii) that comprises an N-terminal portion of the amino acid sequence set forth in SEQ ID NO:7 that is at least 100 amino acid residues in length, (iv) that is at least 85 percent identical to said N-terminal portion, (v) that comprises a C-terminal portion of the amino acid sequence set forth in SEQ ID NO:7 that is at least 20 amino acid residues in length, or (vi) that is at least 85 percent identical to said C-terminal portion;
(7) an amino acid sequence (i) that comprises the amino acid sequence set forth in SEQ ID NO:8, (ii) that is at least 85 percent identical to the amino acid sequence set forth in SEQ ID NO:8, (iii) that comprises an N-terminal portion of the amino acid sequence set forth in SEQ ID NO:8 that is at least 100 amino acid residues in length, (iv) that is at least 85 percent identical to said N-terminal portion, (v) that comprises a C-terminal portion of the amino acid sequence set forth in SEQ ID NO:8 that is at least 20 amino acid residues in length, or (vi) that is at least 85 percent identical to said C-terminal portion;
(8) an amino acid sequence (i) that comprises the amino acid sequence set forth in SEQ ID NO:9, (ii) that is at least 90 percent identical to the amino acid sequence set forth in SEQ ID NO:9, (iii) that comprises an N-terminal portion of the amino acid sequence set forth in SEQ ID NO:9 that is at least 100 amino acid residues in length, (iv) that is at least 93% identical to said N-terminal portion, (v) that comprises a C-terminal portion of the amino acid sequence set forth in SEQ ID NO:9 that is at least 23 amino acid residues in length, or (vi) that is at least 88% identical to said C-terminal portion;
(9) an amino acid sequence (i) that comprises the amino acid sequence set forth in SEQ ID NO:10, (ii) that is at least 90 percent identical to the amino acid sequence set forth in SEQ ID NO:10, (iii) that comprises an N-terminal portion of the amino acid sequence set forth in SEQ ID NO: 10 that is at least 105 amino acid residues in length, (iv) that is at least 94% identical to said N-terminal portion, (v) that comprises a C-terminal portion of the amino acid sequence set forth in SEQ ID NO: 10 that is at least 23 amino acid residues in length, or (vi) that is at least 88% identical to said C-terminal portion;
(10) an amino acid sequence (i) that comprises the amino acid sequence set forth in SEQ ID NO:11, (ii) that is at least 90 percent identical to the amino acid sequence set forth in SEQ ID NO:11, (iii) that comprises an N-terminal portion of the amino acid sequence set forth in SEQ ID NO:11 that is at least 100 amino acid residues in length, (iv) that is at least 93% identical to said N-terminal portion, (v) that comprises a C-terminal portion of the amino acid sequence set forth in SEQ ID NO:11 that is at least 25 amino acid residues in length, or (vi) that is at least 86% identical to said C-terminal portion;
(11) an amino acid sequence (i) that comprises the amino acid sequence set forth in SEQ ID NO:12, (ii) that is at least 90 percent identical to the amino acid sequence set forth in SEQ ID NO:12, (iii) that comprises an N-terminal portion of the amino acid sequence set forth in SEQ ID NO:12 that is at least 108 amino acid residues in length, (iv) that is at least 93% identical to said N-terminal portion, (v) that comprises a C-terminal portion of the amino acid sequence set forth in SEQ ID NO:12 that is at least 23 amino acid residues in length, or (vi) that is at least 85% identical to said C-terminal portion; or
(12) an amino acid sequence (i) that comprises the amino acid sequence set forth in SEQ ID NO:13, (ii) that is at least 90 percent identical to the amino acid sequence set forth in SEQ ID NO:13, (iii) that comprises an N-terminal portion of the amino acid sequence set forth in SEQ ID NO:13 that is at least 116 amino acid residues in length, (iv) that is at least 93% identical to said N-terminal portion, (v) that comprises a C-terminal portion of the amino acid sequence set forth in SEQ ID NO:13 that is at least 18 amino acid residues in length, or (vi) that is at least 85% identical to said C-terminal portion, and
(b) incubating said cell such that said chimeric polypeptide generates a mutation within said mitochondrial or said nucleic genomic DNA.

24. The method of claim 23, wherein said mutation is a single base substitution.

25. The method of claim 23, wherein said introducing comprises introducing nucleic acid encoding said chimeric polypeptide into said cell.

26. The method of claim 23, wherein said nucleic acid interacting domain is N-terminal to said facilitating domain.

27-29. (canceled)

30. The method of claim 23, wherein said chimeric polypeptide further comprises a linker between said nucleic acid interacting domain and said facilitating domain.

31. The method of claim 23, wherein said chimeric polypeptide further comprises a MTS.

32. The method of claim 31, wherein said MTS is at the N-terminus of said chimeric polypeptide.

33. The method of claim 31, wherein said MTS is an isocitrate dehydrogenase 2 MTS, a human COX8A MTS, or a human SOD2 MTS.

34. The method of claim 31, wherein said chimeric polypeptide comprises, in order from N-terminus to C-terminus, said MTS, said nucleic acid interacting domain, and said facilitating domain, and wherein said chimeric polypeptide further comprises a linker between said MTS and said nucleic acid interacting domain.

35-41. (canceled)

42. The method of claim 23, wherein said method comprises introducing a first chimeric polypeptide and a second chimeric polypeptide into said cell, wherein said facilitating domain of said first chimeric polypeptide comprises:
said N-terminal portion of the amino acid sequence set forth in SEQ ID NO:2 that is at least 100 amino acid residues in length, or said amino acid sequence that is at least 92 percent identical to said N-terminal portion;
said N-terminal portion of the amino acid sequence set forth in SEQ ID NO:3 that is at least 100 amino acid residues in length, or said amino acid sequence that is at least 85 percent identical to said N-terminal portion;
said N-terminal portion of the amino acid sequence set forth in SEQ ID NO:4 that is at least 105 amino acid residues in length, or said amino acid sequence that is at least 85 percent identical to said N-terminal portion;
said N-terminal portion of the amino acid sequence set forth in SEQ ID NO:5 that is at least 90 amino acid residues in length, or said amino acid sequence that is at least 85 percent identical to said N-terminal portion;
said N-terminal portion of the amino acid sequence set forth in SEQ ID NO:6 that is at least 110 amino acid residues in length, or said amino acid sequence that is at least 85 percent identical to said N-terminal portion;
said N-terminal portion of the amino acid sequence set forth in SEQ ID NO:7 that is at least 100 amino acid residues in length, or said amino acid sequence that is at least 85 percent identical to said N-terminal portion; or
said N-terminal portion of the amino acid sequence set forth in SEQ ID NO:8 that is at least 100 amino acid residues in length, or said amino acid sequence that is at least 85 percent identical to said N-terminal portion,
and wherein said facilitating domain of said second chimeric polypeptide comprises:
said C-terminal portion of the amino acid sequence set forth in SEQ ID NO:2 that is at least 25 amino acid residues in length, or said amino acid sequence that is at least 92 percent identical to said C-terminal portion;
said C-terminal portion of the amino acid sequence set forth in SEQ ID NO:3 that is at least 25 amino acid residues in length, or said amino acid sequence that is at least 85 percent identical to said C-terminal portion;
said C-terminal portion of the amino acid sequence set forth in SEQ ID NO:4 that is at least 25 amino acid residues in length, or said amino acid sequence that is at least 85 percent identical to said C-terminal portion;
said C-terminal portion of the amino acid sequence set forth in SEQ ID NO:5 that is at least 20 amino acid residues in length, or said amino acid sequence that is at least 85 percent identical to said C-terminal portion;
said C-terminal portion of the amino acid sequence set forth in SEQ ID NO:6 that is at least 20 amino acid residues in length, or said amino acid sequence that is at least 85 percent identical to said C-terminal portion;
said C-terminal portion of the amino acid sequence set forth in SEQ ID NO:7 that is at least 20 amino acid residues in length, or said amino acid sequence that is at least 85 percent identical to said C-terminal portion; or
said C-terminal portion of the amino acid sequence set forth in SEQ ID NO:8 that is at least 20 amino acid residues in length, or said amino acid sequence that is at least 85 percent identical to said C-terminal portion.

43. The method of claim 42, wherein said method comprises introducing a first chimeric polypeptide and a second chimeric polypeptide into said cell, and
wherein said facilitating domain of said first chimeric polypeptide comprises said N-terminal portion of the amino acid sequence set forth in SEQ ID NO:2 that is at least 100 amino acid residues in length, or said amino acid sequence that is at least 92 percent identical to said N-terminal portion, and wherein said facilitating domain of said second chimeric polypeptide comprises said C-terminal portion of the amino acid sequence set forth in SEQ ID NO:2 that is at least 25 amino acid residues in length, or said amino acid sequence that is at least 92 percent identical to said C-terminal portion; or
wherein said facilitating domain of said first chimeric polypeptide comprises said N-terminal portion of the amino acid sequence set forth in SEQ ID NO:3 that is at least 100 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:3 that is at least 25 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:4 that is at least 105 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:4 that is at least 25 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:5 that is at least 90 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:5 that is at least 20 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:6 that is at least 110 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:6 that is at least 20 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:7 that is at least 100 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:7 that is at least 20 amino acid residues in length, or said amino acid sequence that is at least 85 percent identical to said C-terminal portion; or
wherein said facilitating domain of said first chimeric polypeptide comprises said N-terminal portion of the amino acid sequence set forth in SEQ ID NO:8 that is at least 100 amino acid residues in length, or said amino acid sequence that is at least 85 percent identical to said N-terminal portion, and wherein said facilitating domain of said second chimeric polypeptide comprises said C-terminal portion of the amino acid sequence set forth in SEQ ID NO:8 that is at least 20 amino acid residues in length, or said amino acid sequence that is at least 85 percent identical to said C-terminal portion.
